(19) United States
(12) Patent Application Publication
(10) Pub. No.: US 2009/0324602 A1
(43) Pub. Date: Dec. 31, 2009

(54) ANTI-FN14 ANTIBODIES AND USES THEREOF

(75) Inventors: Ellen Garber, Cambridge, MA (US); Linda Burkly, West Newton, MA (US); Jennifer Michaelson, Brighton, MA (US); Alexey Lugovskoy, Woburn, MA (US); Yen-Ming Hsu, Lexington, MA (US); Karl Hanf, Billerica, MA (US)

Correspondence Address: FISH & RICHARDSON PC, P.O. BOX 1022, MINNEAPOLIS, MN, 55440-1022, US

(73) Assignee: Biogen Idec MA Inc., Cambridge, MA (US)

(21) Appl. No.: 12/463,291

(22) Filed: May 8, 2009

Related U.S. Application Data
(60) Provisional application No. 61/173,137, filed on Apr. 27, 2009.
(60) Provisional application No. 61/149,517, filed on Feb. 3, 2009.
(60) Provisional application No. 61/053,650, filed on May 15, 2008.

(51) Int. Cl. A61K 39/395; C07K 16/00; C12N 5/16; C12N 5/02

(52) U.S. Cl. 424/139.1; 530/387.9; 530/387.3; 435/326; 424/133.1; 435/375

(57) ABSTRACT

Antibodies and antibody fragments that bind to the receptor Fn14 and induce or enhance cell killing of Fn14-expressing cancer cells are disclosed. Also disclosed are methods of using the antibodies and antibody fragments to induce death of a tumor cell and treat disorders and in a subject.

### CROSS REFERENCE TO RELATED APPLICATIONS

This application claims priority from provisional application No. 61/053,650, filed May 15, 2008, provisional application No. 61/149,517, filed Feb. 3, 2009, and provisional application No. 61/173,137, filed Apr. 27, 2009. The entire content of each of these prior applications is incorporated herein by reference in its entirety.

### BACKGROUND

The tumor-necrosis factor (TNF)-related cytokines are a superfamily of proteins that have an array of functions, including ones implicated in immune regulation and apoptosis regulation. TWEAK (TNF-like weak inducer of apoptosis) is one member of this superfamily. Fn14, a TWEAK receptor, is a growth factor-regulated immediate-early response gene that decreases cellular adhesion to the extracellular matrix and reduces serum-stimulated growth and migration (Meighan-Mantha et al., J. Biol. Chem. 274:33166-33176 (1999)).

### SUMMARY

The invention is based, at least in part, on the identification and characterization of antibodies that bind to Fn14 and induce death of tumor cells. The antibodies are effective in animal models of cancer at low doses and with a prolonged effect in preventing tumor growth.

In one aspect, the invention features an isolated Fn14-binding protein (e.g., an isolated antibody or antigen-binding fragment thereof) that (i) selectively binds to the polypeptide of SEQ ID NO:1, when expressed on the surface of a cell, at an epitope that includes the amino acid residue tryptophan at position 42 of SEQ ID NO:1, and (ii) induces or enhances cell killing of cancer cells (e.g., WiDr colon cancer cells) in vivo or in vitro. The term “selectively binds” refers to binding of the Fn14-binding protein to its target protein (e.g., the polypeptide of SEQ ID NO:1) in a manner that exhibits specificity to the target protein when present in a population of heterogeneous proteins (i.e., “selective” binding does not encompass non-specific protein-protein interactions).

As used herein, binding “at an epitope that includes the amino acid residue tryptophan at position 42 of SEQ ID NO:1” refers to the ability of an antibody or antigen-binding fragment thereof to selectively bind to the wild-type human Fn14 protein of SEQ ID NO:1 but the inability to significantly bind to a mutant of SEQ ID NO:1 that contains an alanine substituted for tryptophan at position 42. For example, binding to a mutant of SEQ ID NO:1 that contains an alanine substituted for tryptophan at position 42 may occur at a level that is less than 50%, less than 40%, less than 30%, less than 20%, or less than 10% the level of binding that occurs to the wild-type human Fn14 protein of SEQ ID NO:1 under the same assay conditions.

Also disclosed is an isolated Fn14-binding protein (e.g., an isolated antibody or antigen-binding fragment thereof) that (i) selectively binds to the polypeptide of SEQ ID NO:1, when expressed on the surface of a cell, and crossblocks binding of the monoclonal antibody P4A8, P2D3, or P3G5 to SEQ ID NO:1, and (ii) induces or enhances cell killing of cancer cells (e.g., WiDr colon cancer cells) in vivo or in vitro.

An Fn14-binding protein crossblocks binding of a monoclonal antibody (e.g., P4A8 or P3G5 or P2D3) to Fn14 when the Fn14-binding protein's prior binding to Fn14 inhibits later binding of the monoclonal antibody to Fn14 at the same level at which the monoclonal antibody's prior binding to Fn14 inhibits later binding of the identical monoclonal antibody to Fn14. For example, an Fn14-binding protein crossblocks binding of P4A8 to Fn14 when the Fn14-binding protein's prior binding to Fn14 inhibits later binding of P4A8 to Fn14 at the same level at which P4A8's prior binding to Fn14 inhibits later binding of the identical monoclonal antibody to Fn14. In certain embodiments, an Fn14-binding protein crossblocks the binding of P4A8 to human Fn14 to a level that is at least about 30%, 50%, 70%, 80%, 90%, 95%, 98% or 99% of crossblocking achieved by P4A8 of itself. In certain embodiments, an Fn14-binding protein crossblocks the binding of P4A8 to human Fn14 to a higher degree than another anti-Fn14 antibody (e.g., ITEM-1, ITEM-2, ITEM-3 or ITEM-4) crossblocks the binding of P4A8 to human Fn14.

In certain embodiments, P4A8 crossblocks the binding of an Fn14-binding protein to human Fn14 to a level that is at least about 30%, 50%, 70%, 80%, 90%, 95%, 98% or 99% of crossblocking achieved by the Fn14-binding protein of itself.

In certain embodiments, (i) an Fn14-binding protein crossblocks the binding of P4A8 to human Fn14 and (ii) P4A8 crossblocks the binding of the Fn14-binding protein to human Fn14. Complete crossblocking both ways indicates that the two antibodies have the same footprint, i.e., bind to the same epitope. In certain embodiments, crossblocking one way or both ways is not complete, but partial, e.g., to a level that is at least about 30%, 50%, 70%, 80%, 90%, 95%, 98% or 99% of crossblocking achieved by the antibody itself. A partial crossblocking one way or both ways indicates that the footprints of the two antibodies are not identical, but may be overlapping or in close proximity.

The binding of Fn14-binding proteins can also be described as set forth above but relative to P3G5 or P2D3, instead of P4A8. Crossblocking experiments may be conducted with the test Fn14-binding protein being present at or above saturating concentrations for Fn14 binding based on its binding affinity.

In certain embodiments, an Fn-14-binding protein binds to the same epitope or substantially the same epitope as that of P4A8, P3G5, or P2D3, as characterized by one or more of the experiments described herein, e.g., crossblocking experiments and the binding experiments to various Fn14 species and mutated Fn14 proteins.

Also disclosed is an isolated Fn14-binding protein (e.g., an isolated antibody or antigen-binding fragment thereof) that (i) selectively binds to the polypeptide of SEQ ID NO:1, when expressed on the surface of a cell, and crossblocks binding to SEQ ID NO:1 of a monoclonal antibody comprising the VH and VL domains of P4A8, a monoclonal antibody comprising the VH and VL domains of P3G5, or a monoclonal antibody comprising the VH and VL domains of P2D3, and (ii) induces or enhances cell killing of cancer cells (e.g., WiDr colon cancer cells) in vivo or in vitro.

Also disclosed is an isolated antibody or antigen-binding fragment thereof that (i) selectively binds to the polypeptide of SEQ ID NO:1, when expressed on the surface of a cell, (ii) comprises a mutation in a constant region of the antibody that results in reduced or absent effector function, and (iii) induces or enhances cell killing of cancer cells (e.g., WiDr colon cancer cells) in vivo or in vitro. In some embodiments, the antibody or antigen-binding fragment thereof binds to the polypeptide of SEQ ID NO:1 at an epitope that includes the amino acid residue tryptophan at position 42 of SEQ ID NO:1.

The term “effector function” refers to the functional ability of the Fc or constant region of an antibody to bind proteins and/or cells of the immune system. Antibodies having reduced effector function and methods for engineering such antibodies are well-known in the art (see, e.g., WO 05/18572, WO 05/03175, and U.S. Pat. No. 6,242,195) and are described in further detail herein. Typical effector functions include the ability to bind complement protein (e.g., the complement protein C1q), and/or an Fc receptor (FcR) (e.g., FcγRI, FcγRII, FcγRIIa, FcγRIIb, FcγRIII, FcγRIIIa, and/or FcγRIIIb). The functional consequences of being able to bind one or more of the foregoing molecules include, without limitation, opsonization, phagocytosis, antigen-dependent cellular cytotoxicity (ADCC), complement-dependent cytotoxicity (CDC) and/or effector cell modulation. A decrease in effector function refers to a decrease in one or more of the biochemical or cellular activities induced at least in part by binding of Fc to its cognate receptor or to a complement protein or effector cell, while maintaining the antigen-binding activity of the variable region of the antibody (or fragment thereof), albeit with reduced, similar, identical, or increased binding affinity. Decreases in effector function, e.g., Fc binding to an Fc receptor or complement protein, can be expressed in terms of fold reduction (e.g., reduced by 1.5-fold, 2-fold, and the like) and may be calculated based on, e.g., the percent reductions in binding activity determined using binding assays known in the art (see, for example, WO 05/18572). Fc-mediated receptor hypercrosslinking can also be a factor that enhances activity.

Also disclosed is an isolated Fn14-binding protein (e.g., an isolated antibody or antigen-binding fragment thereof) that (i) selectively binds to the polypeptide of SEQ ID NO:1, when expressed on the surface of a cell, at the same epitope as the monoclonal antibody P4A8, P3G5, or P2D3 (or a monoclonal antibody comprising the VH and VL domains of P4A8, a monoclonal antibody comprising the VH and VL domains of P3G5, or a monoclonal antibody comprising the VH and VL domains of P2D3), and (ii) induces or enhances cell killing of cancer cells (e.g., WiDr colon cancer cells) in vivo or in vitro.

In some embodiments, binding of an Fn14-binding protein (e.g., an isolated antibody or antigen-binding fragment thereof) described herein to the polypeptide of SEQ ID NO:1 blocks or decreases binding of TWEAK to the polypeptide. Binding may be decreased by a factor of at least about 10%, 30%, 50%, 70%, 80%, 90%, 95%, or 100%. TWEAK binding to FN14 can be measured in various cell based systems. For example, cells can be transfected with a vector encoding Fn14 and TWEAK binding to the transfected cells can be measured by contacting the cells with a soluble TWEAK protein linked to a detectable marker. An Fn14-binding protein can be added to the cells prior to addition of the soluble TWEAK protein to determine whether the Fn14-binding protein blocks or decreases binding of TWEAK to Fn14.

Also disclosed herein is an isolated Fn14-binding protein (e.g., an isolated antibody or antigen-binding fragment thereof) that selectively binds to the polypeptide of SEQ ID NO:1, when expressed on the surface of a cell, and that mimics at least one biological activity resulting from binding of TWEAK to Fn14, e.g., induction of IL-8, induction of cleavage of a caspase, and/or induction of NFkB activity (e.g., an agonist antibody).

Further disclosed herein is an isolated Fn14-binding protein (e.g., an isolated antibody or antigen-binding fragment thereof) that selectively binds to the polypeptide of SEQ ID NO:1, when expressed on the surface of a cell, and that also binds significantly (or detectably) to cynomolgus, mouse and rat Fn14.

Also disclosed herein is an isolated Fn14-binding protein (e.g., an isolated antibody or antigen-binding fragment thereof) that selectively binds to the polypeptide of SEQ ID NO:1, when expressed on the surface of a cell, and is internalized into the cell following its binding to Fn14 on the surface of the cell.

Antibodies or antigen binding fragments thereof that selectively bind to the polypeptide of SEQ ID NO:1, when expressed on the surface of a cell, and that kill tumor cells include antibodies having any combination of characteristics described herein, e.g., (i) agonist activity or mimicking of at least some of the biologic effects resulting from binding of TWEAK to Fn14, (ii) significant blocking of TWEAK binding to Fn14, (iii) binding to an epitope of human Fn14 that includes W42, (iv) significant binding to human, cynomolgus, rat and mouse Fn14, and (iv) lack of or reduced induction of at least some effector functions. For example, in one embodiment, an Fn14 antibody is an agonist antibody that blocks TWEAK binding to Fn14. The antibody may further bind to an epitope of Fn14 encompassing W42 and/or have an Fc region that has reduced effector function.

In certain embodiments, an isolated Fn14-binding protein (e.g., an isolated antibody or antigen-binding fragment thereof) that selectively binds to the polypeptide of SEQ ID NO:1, when expressed on the surface of a cell, and induces or enhances cell killing is not an antibody that is known in the art, e.g., ITEM-1, ITEM-2, ITEM-3 or ITEM-4, as described, e.g., in Nakayama et al. (2003) J. Immunol. 170: 341, Nakayama et al. (2003) BBRC 306:819 and Harada et al. (2002) BBRC 299:488.

In certain embodiments, the antibody or antigen binding fragment thereof has dissociation kinetics in the range of 10⁻² to 10⁻⁶ s⁻¹, typically 10⁻² to 10⁻⁵ s⁻¹, e.g., 10⁻² to 10⁻³ s⁻¹, such as 1×10⁻³ to 5×10⁻³ s⁻¹ (see also Example 14). In one embodiment, the antibody binds to human Fn14, with an affinity and/or kinetics similar to (e.g., within a factor of five or ten of) monoclonal antibody P4A8, or modified forms thereof, e.g., chimeric forms or humanized forms thereof (e.g., a humanized form described herein). The affinity and binding kinetics of the anti-Fn14 antibody can be tested, e.g., using biosensor technology (BIACORE™).

In certain embodiments, the antibody or antigen binding fragment thereof has dissociation kinetics in the range of 10⁻² to 10⁻⁶ s⁻¹, typically 10⁻² to 10⁻⁵ s⁻¹. In one embodiment, the antibody binds to human Fn14, with an affinity and/or kinetics similar to (e.g., within a factor of five or ten of) monoclonal antibody P2D3, or modified forms thereof, e.g., chimeric forms or humanized forms thereof (e.g., a humanized form described herein).

In certain embodiments, the antibody or antigen binding fragment thereof has dissociation kinetics in the range of 10⁻² to 10⁻⁶ s⁻¹, typically 0-2 to 10⁻⁵ s⁻¹. In one embodiment, the antibody binds to human Fn14, with an affinity and/or kinetics similar to (e.g., within a factor of five or ten of) monoclonal antibody P3G5, or modified forms thereof, e.g., chimeric forms or humanized forms thereof (e.g., a humanized form described herein).

In certain embodiments, the antibody or antigen binding fragment thereof has association kinetics in the range of 10⁵ to 10⁷ M⁻¹s⁻¹, such as 5×10⁵ to 5×10⁶ M⁻¹s⁻¹, e.g., 7×10⁵ to 3×10⁶ M⁻¹s⁻¹ (see Example 14). In certain embodiments, the antibody or antigen binding fragment thereof has an association constant of 10⁵ to 10⁷ M⁻¹s⁻¹, such as 5×10⁵ to 5×10⁶ M⁻¹s⁻¹, e.g., 7×10⁵ to 3×10⁶ M⁻¹s⁻¹ and a dissociation constant of 10⁻² to 10⁻³ s⁻¹, such as 1×10⁻³ to 5×10⁻³ s⁻¹. Antibodies or antigen binding fragments thereof may have an affinity constant of 10⁻¹⁰, 10⁻⁹ or 10⁻⁸ M or lower, such as in the range of 10⁻¹⁰ M to 10⁻⁹, e.g., 5×10⁻¹ to 5×10⁻⁹ M or 1×10⁻⁹ to 5×10⁻⁹ M (see Example 14). These kinetic parameters may be characteristic of binding of the antibody or antigen binding fragment thereof to a soluble Fn14 protein, such as soluble human Fn14 protein, e.g., consisting essentially of the extracellular or cysteine rich region of human Fn14 (e.g., about amino acids 28-68, 69, 70 or 80, or from about amino acid 28 to about an amino acid from amino acid 68 to 80 of human Fn14).

In certain embodiments, the antibody or antigen binding fragment thereof interacts with one or more of residues C49, W42, K48, D51, R58, A57, H60, R56, L46, and M50 of human Fn14.

In certain embodiments, an anti-Fn14 antibody binds substantially to the same epitope as that to which P4A8, P3G5 or P2D3 binds. Whether two antibodies bind substantially to the same epitope can be determined by a competition assay. Such an assay may be conducted by labeling a control antibody (e.g., P4A8 or other antibody described herein) with a detectable label, such as biotin. The intensity of the bound label to Fn14 is measured. If the labeled antibody competes with the unlabeled (test antibody) by binding to an overlapping epitope, the intensity will be decreased relative to the binding by negative control unlabeled antibody.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain that is at least 80% identical to the amino acid sequence of SEQ ID NO:2, SEQ ID NO:3, or SEQ ID NO:4, and (iii) induces or enhances cell killing of cancer cells (e.g., WiDr colon cancer cells) in vivo or in vitro. In some embodiments, the VH domain is at least 90% identical to the amino acid sequence of SEQ ID NO:2, SEQ ID NO:3, or SEQ ID NO:4. In some embodiments, the VH domain is at least 95% identical to the amino acid sequence of SEQ ID NO:2, SEQ ID NO:3, or SEQ ID NO:4. In some embodiments, the VH domain is identical to the amino acid sequence of SEQ ID NO:2, SEQ ID NO:3, or SEQ ID NO:4.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VL domain that is at least 80% identical to the amino acid sequence of SEQ ID NO:5, SEQ ID NO:6, or SEQ ID NO:7, and (iii) induces or enhances cell killing of cancer cells (e.g., WiDr colon cancer cells) in vivo or in vitro. In some embodiments, the VL domain is at least 90% identical to the amino acid sequence of SEQ ID NO:5, SEQ ID NO:6, or SEQ ID NO:7. In some embodiments, the VL domain is at least 95% identical to the amino acid sequence of SEQ ID NO:5, SEQ ID NO:6, or SEQ ID NO:7. In some embodiments, the VL domain is identical to the amino acid sequence of SEQ ID NO:5, SEQ ID NO:6, or SEQ ID NO:7.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain that is at least 80% identical to the amino acid sequence of SEQ ID NO:2, SEQ ID NO:3, or SEQ ID NO:4, (iii) comprises a VL domain that is at least 80% identical to the amino acid sequence of SEQ ID NO:5, SEQ ID NO:6, or SEQ ID NO:7, and (iv) induces or enhances cell killing of cancer cells (e.g., WiDr colon cancer cells) in vivo or in vitro. In some embodiments, (i) the VH domain is at least 90% identical to the amino acid sequence of SEQ ID NO:2, SEQ ID NO:3, or SEQ ID NO:4, and (ii) the VL domain is at least 90% identical to the amino acid sequence of SEQ ID NO:5, SEQ ID NO:6, or SEQ ID NO:7. In some embodiments, (i) the VH domain is at least 95% identical to the amino acid sequence of SEQ ID NO:2, SEQ ID NO:3, or SEQ ID NO:4, and (ii) the VL domain is at least 95% identical to the amino acid sequence of SEQ ID NO:5, SEQ ID NO:6, or SEQ ID NO:7. In some embodiments, (i) the VH domain is identical to the amino acid sequence of SEQ ID NO:2, SEQ ID NO:3, or SEQ ID NO:4, and (ii) the VL domain is identical to the amino acid sequence of SEQ ID NO:5, SEQ ID NO:6, or SEQ ID NO:7.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain that is at least 80% identical to the amino acid sequence of SEQ ID NO:11 or SEQ ID NO:12, and (iii) induces or enhances cell killing of cancer cells (e.g., WiDr colon cancer cells) in vivo or in vitro. In some embodiments, the VH domain is at least 90% identical to the amino acid sequence of SEQ ID NO:11 or SEQ ID NO:12. In some embodiments, the VH domain is at least 95% identical to the amino acid sequence of SEQ ID NO:11 or SEQ ID NO:12. In some embodiments, the VH domain is identical to the amino acid sequence of SEQ ID NO:11 or SEQ ID NO:12.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VL domain that is at least 80% identical to the amino acid sequence of SEQ ID NO:13, SEQ ID NO:14, or SEQ ID NO:15, and (iii) induces or enhances cell killing of cancer cells (e.g., WiDr colon cancer cells) in vivo or in vitro. In some embodiments, the VL domain is at least 90% identical to the amino acid sequence of SEQ ID NO:13, SEQ ID NO:14, or SEQ ID NO:15. In some embodiments, the VL domain is at least 95% identical to the amino acid sequence of SEQ ID NO:13, SEQ ID NO:14, or SEQ ID NO:15. In some embodiments, the VL domain is identical to the amino acid sequence of SEQ ID NO:13, SEQ ID NO:14, or SEQ ID NO:15.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain that is at least 80% identical to the amino acid sequence of SEQ ID NO:111 or SEQ ID NO:12, (iii) comprises a VL domain that is at least 80% identical to the amino acid sequence of SEQ ID NO:13, SEQ ID NO:14, or SEQ ID NO:15, and (iv) induces or enhances cell killing of cancer cells (e.g., WiDr colon cancer cells) in vivo or in vitro. In some embodiments, (i) the VH domain is at least 90% identical to the amino acid sequence of SEQ ID NO:11 or SEQ ID NO:12, and (ii) the VL domain is at least 90% identical to the amino acid sequence of SEQ ID NO:13, SEQ ID NO:14, or SEQ ID NO:15. In some embodiments, (i) the VH domain is at least 95% identical to the amino acid sequence of SEQ ID NO:11 or SEQ ID NO:12, and (ii) the VL domain is at least 95% identical to the amino acid sequence of SEQ ID NO:13, SEQ ID NO:14, or SEQ ID NO:15. In some embodiments, (i) the VH domain is identical to the amino acid sequence of SEQ ID NO:11 or SEQ ID NO:12, and (ii) the VL domain is identical to the amino acid sequence of SEQ ID NO:13, SEQ ID NO:14, or SEQ ID NO:15. In some embodiments, the heavy chain comprises SEQ ID NO:37 or SEQ ID NO:39 and the light chain comprises SEQ ID NO:41, SEQ ID NO:43, or SEQ ID NO:45. In some embodiments, the heavy chain comprises SEQ ID NO:37 and the light chain comprises SEQ ID NO:43.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising (a) a first heavy chain complementarity determining region (CDR) that is at least 90% identical to CDR-H1 of SEQ ID NO:2 or SEQ ID NO:3, a second heavy chain CDR that is at least 90% identical to CDR-H2 of SEQ ID NO:2 or SEQ ID NO:3, and a third heavy chain CDR that is at least 90% identical to CDR-H3 of SEQ ID NO:2 or SEQ ID NO:3, or (b) a first heavy chain CDR that is at least 90% identical to CDR-H1 of SEQ ID NO:4, a second heavy chain CDR that is at least 90% identical to CDR-H2 of SEQ ID NO:4, and a third heavy chain CDR that is at least 90% identical to CDR-H3 of SEQ ID NO:4, and (iii) induces or enhances cell killing of cancer cells (e.g., WiDr colon cancer cells) in vivo or in vitro. In some embodiments, the first heavy chain CDR is identical to CDR-H1 of SEQ ID NO:2 or SEQ ID NO:3, the second heavy chain CDR is identical to CDR-H2 of SEQ ID NO:2 or SEQ ID NO:3, and the third heavy chain CDR is identical to CDR-H3 of SEQ ID NO:2 or SEQ ID NO:3. In some embodiments, the first heavy chain CDR is identical to CDR-H1 of SEQ ID NO:4, the second heavy chain CDR is identical to CDR-H2 of SEQ ID NO:4, and the third heavy chain CDR is identical to CDR-H3 of SEQ ID NO:4.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VL domain comprising (a) a first light chain CDR that is at least 90% identical to CDR-L1 of SEQ ID NO:5 or SEQ ID NO:6, a second light chain CDR that is at least 90% identical to CDR-L2 of SEQ ID NO:5 or SEQ ID NO:6, and a third light chain CDR that is at least 90% identical to CDR-L3 of SEQ ID NO:5 or SEQ ID NO:6, or (b) a first light chain CDR that is at least 90% identical to CDR-L1 of SEQ ID NO:7, a second light chain CDR that is at least 90% identical to CDR-L2 of SEQ ID NO:7, and a third light chain CDR that is at least 90% identical to CDR-L3 of SEQ ID NO:7, and (iii) induces or enhances cell killing of cancer cells (e.g., WiDr colon cancer cells) in vivo or in vitro. In some embodiments, the first light chain CDR is identical to CDR-L1 of SEQ ID NO:5 or SEQ ID NO:6, the second light chain CDR is identical to CDR-L2 of SEQ ID NO:5 or SEQ ID NO:6, and the third light chain CDR is identical to CDR-L3 of SEQ ID NO:5 or SEQ ID NO:6. In some embodiments, the first light chain CDR is identical to CDR-L1 of SEQ ID NO:7, the second light chain CDR is identical to CDR-L2 of SEQ ID NO:7, and the third light chain CDR is identical to CDR-L3 of SEQ ID NO:7.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising (a) a first heavy chain CDR that is at least 90% identical to CDR-H1 of SEQ ID NO:2 or SEQ ID NO:3, a second heavy chain CDR that is at least 90% identical to CDR-H2 of SEQ ID NO:2 or SEQ ID NO:3, and a third heavy chain CDR that is at least 90% identical to CDR-H3 of SEQ ID NO:2 or SEQ ID NO:3, or (b) a first heavy chain CDR that is at least 90% identical to CDR-H1 of SEQ ID NO:4, a second heavy chain CDR that is at least 90% identical to CDR-H2 of SEQ ID NO:4, and a third heavy chain CDR that is at least 90% identical to CDR-H3 of SEQ ID NO:4, (iii) comprises a VL domain comprising (a) a first light chain CDR that is at least 90% identical to CDR-L1 of SEQ ID NO:5 or SEQ ID NO:6, a second light chain CDR that is at least 90% identical to CDR-L2 of SEQ ID NO:5 or SEQ ID NO:6, and a third light chain CDR that is at least 90% identical to CDR-L3 of SEQ ID NO:5 or SEQ ID NO:6, or (b) a first light chain CDR that is at least 90% identical to CDR-L1 of SEQ ID NO:7, a second light chain CDR that is at least 90% identical to CDR-L2 of SEQ ID NO:7, and a third light chain CDR that is at least 90% identical to CDR-L3 of SEQ ID NO:7, and (iv) induces or enhances cell killing of cancer cells (e.g., WiDr colon cancer cells) in vivo or in vitro. In some embodiments, (i) the first heavy chain CDR is identical to CDR-H1 of SEQ ID NO:2, the second heavy chain CDR is identical to CDR-H2 of SEQ ID NO:2, and the third heavy chain CDR is identical to CDR-H3 of SEQ ID NO:2, and (ii) the first light chain CDR is identical to CDR-L1 of SEQ ID NO:5, the second light chain CDR is identical to CDR-L2 of SEQ ID NO:5, and the third light chain CDR is identical to CDR-L3 of SEQ ID NO:5. In some embodiments, (i) the first heavy chain CDR is identical to CDR-H1 of SEQ ID NO:3, the second heavy chain CDR is identical to CDR-H2 of SEQ ID NO:3, and the third heavy chain CDR is identical to CDR-H3 of SEQ ID NO:3, and (ii) the first light chain CDR is identical to CDR-L1 of SEQ ID NO:6, the second light chain CDR is identical to CDR-L2 of SEQ ID NO:6, and the third light chain CDR is identical to CDR-L3 of SEQ ID NO:6. In some embodiments, (i) the first heavy chain CDR is identical to CDR-H1 of SEQ ID NO:4, the second heavy chain CDR is identical to CDR-H2 of SEQ ID NO:4, and the third heavy chain CDR is identical to CDR-H3 of SEQ ID NO:4, and (ii) the first light chain CDR is identical to CDR-L1 of SEQ ID NO:7, the second light chain CDR is identical to CDR-L2 of SEQ ID NO:7, and the third light chain CDR is identical to CDR-L3 of SEQ ID NO:7. In some embodiments, VH domain comprises amino acids 1-121 of SEQ ID NO:8. In some embodiments, VL domain comprises amino acids 1-111 of SEQ ID NO:9. In some embodiments, VH domain comprises amino acids 1-121 of SEQ ID NO: 8 and the VL domain comprises amino acids 1-111 of SEQ ID NO:9. In some embodiments, the heavy chain comprises SEQ ID NO:8 and the light chain comprises SEQ ID NO:9. In some embodiments, the heavy chain comprises SEQ ID NO:16. In some embodiments, the heavy chain comprises SEQ ID NO:16 and the light chain comprises SEQ ID NO:9.

An antibody or antigen-binding fragment thereof described herein can optionally contain framework regions that are collectively at least 90% identical (or at least 95, 98, or 99% identical) to human germline framework regions. The term “collectively” means that all frameworks are considered together in the sequence comparison, rather than individual framework regions. For example, an antibody or antigen-binding fragment thereof described herein can comprise VH domain framework regions that are collectively at least 90% identical (or at least 95, 98, or 99% identical) to the framework regions of the VH domain of SEQ ID NO:11 or SEQ ID NO:12. In another example, an antibody or antigen-binding fragment thereof described herein can comprise VL domain framework regions that are collectively at least 90% identical (or at least 95, 98, or 99% identical) to the framework regions of the VL domain of SEQ ID NO:13, SEQ ID NO:14, or SEQ ID NO:15. In some cases, an antibody or antigen-binding fragment thereof described herein can comprise (i) VH domain framework regions that are collectively at least 90% identical to the framework regions of the VH domain of SEQ ID NO:11 or SEQ ID NO:12, and (ii) VL domain framework regions that are collectively at least 90% identical to the framework regions of the VL domain of SEQ ID NO:13, SEQ ID NO:14, or SEQ ID NO:15.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising SEQ ID NO:11, and (iii) comprises a VL domain comprising SEQ ID NO:13.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising CDRs that are identical to the CDRs of SEQ ID NO:11 or wherein each CDR differs from the corresponding CDR of SEQ ID NO:11 in at most one, two, three, or four alterations (e.g., substitutions, deletions, or insertions), wherein the framework regions are collectively at least 90, 95, 97, 98, or 99% identical to the framework regions of SEQ ID NO:11, and (iii) comprises a VL domain comprising CDRs that are identical to the CDRs of SEQ ID NO:13 or wherein each CDR differs from the corresponding CDR of SEQ ID NO:13 in at most one, two, three, or four alterations (e.g., substitutions, deletions, or insertions), wherein the framework regions are collectively at least 90, 95, 97, 98, or 99% identical to the framework regions of SEQ ID NO:13.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising SEQ ID NO:11, and (iii) comprises a VL domain comprising SEQ ID NO:14.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising CDRs that are identical to the CDRs of SEQ ID NO:11 or differ from the CDRs of SEQ ID NO:11 in at most one, two, three, or four alterations (e.g., substitutions, deletions, or insertions), wherein the framework regions are collectively at least 90, 95, 97, 98, or 99% identical to the framework regions of SEQ ID NO:11, and (iii) comprises a VL domain comprising CDRs that are identical to the CDRs of SEQ ID NO:14 or differ from the CDRs of SEQ ID NO:14 in at most one, two, three, or four alterations (e.g., substitutions, deletions, or insertions), wherein the framework regions are collectively at least 90, 95, 97, 98, or 99% identical to the framework regions of SEQ ID NO:14.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising SEQ ID NO:11, and (iii) comprises a VL domain comprising SEQ ID NO:15.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising CDRs that are identical to the CDRs of SEQ ID NO:11 or differ from the CDRs of SEQ ID NO:11 in at most one, two, three, or four alterations (e.g., substitutions, deletions, or insertions), wherein the framework regions are collectively at least 90, 95, 97, 98, or 99% identical to the framework regions of SEQ ID NO:11, and (iii) comprises a VL domain comprising CDRs that are identical to the CDRs of SEQ ID NO:15 or differ from the CDRs of SEQ ID NO:15 in at most one, two, three, or four alterations (e.g., substitutions, deletions, or insertions), wherein the framework regions are collectively at least 90, 95, 97, 98, or 99% identical to the framework regions of SEQ ID NO:15.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising SEQ ID NO:12, and (iii) comprises a VL domain comprising SEQ ID NO:13.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising CDRs that are identical to the CDRs of SEQ ID NO:12 or differ from the CDRs of SEQ ID NO:12 in at most one, two, three, or four alterations (e.g., substitutions, deletions, or insertions), wherein the framework regions are collectively at least 90, 95, 97, 98, or 99% identical to the framework regions of SEQ ID NO:12, and (iii) comprises a VL domain comprising CDRs that are identical to the CDRs of SEQ ID NO:13 or differ from the CDRs of SEQ ID NO:13 in at most one, two, three, or four alterations (e.g., substitutions, deletions, or insertions), wherein the framework regions are collectively at least 90, 95, 97, 98, or 99% identical to the framework regions of SEQ ID NO:13.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising SEQ ID NO:12, and (iii) comprises a VL domain comprising SEQ ID NO:14.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising CDRs that are identical to the CDRs of SEQ ID NO:12 or differ from the CDRs of SEQ ID NO:12 in at most one, two, three, or four alterations (e.g., substitutions, deletions, or insertions), wherein the framework regions are collectively at least 90, 95, 97, 98, or 99% identical to the framework regions of SEQ ID NO:12, and (iii) comprises a VL domain comprising CDRs that are identical to the CDRs of SEQ ID NO:14 or differ from the CDRs of SEQ ID NO:14 in at most one, two, three, or four alterations (e.g., substitutions, deletions, or insertions), wherein the framework regions are collectively at least 90, 95, 97, 98, or 99% identical to the framework regions of SEQ ID NO:14.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising SEQ ID NO:12, and (iii) comprises a VL domain comprising SEQ ID NO:15.

Also disclosed is an isolated Fn14-binding protein (e.g., an isolated antibody or antigen-binding fragment thereof) that (i) selectively binds to the polypeptide of SEQ ID NO:1, when expressed on the surface of a cell, (ii) comprises a VH domain comprising CDRs that are identical to the CDRs of SEQ ID NO:12 or differ from the CDRs of SEQ ID NO:12 in at most one, two, three, or four alterations (e.g., substitutions, deletions, or insertions), wherein the framework regions are collectively at least 90, 95, 97, 98, or 99% identical to the framework regions of SEQ ID NO:12, and (iii) comprises a VL domain comprising CDRs that are identical to the CDRs of SEQ ID NO:15 or differ from the CDRs of SEQ ID NO:15 in at most one, two, three, or four alterations (e.g., substitutions, deletions, or insertions), wherein the framework regions are collectively at least 90, 95, 97, 98, or 99% identical to the framework regions of SEQ ID NO:15.

In one embodiment, the antibody or antigen-binding fragment includes three or all six CDRs from P4A8 or closely related CDRs, e.g., CDRs that are identical or have at least one amino acid alteration, but not more than two, three or four alterations (e.g., substitutions, deletions, or insertions), or other CDR described herein.

In one embodiment, the antibody or antigen-binding fragment includes three or all six CDRs from P3G5 or closely related CDRs, e.g., CDRs that are identical or have at least one amino acid alteration, but not more than two, three or four alterations (e.g., substitutions, deletions, or insertions), or other CDR described herein.

In one embodiment, the antibody or antigen-binding fragment includes three or all six CDRs from P2D3 or closely related CDRs, e.g., CDRs that are identical or have at least one amino acid alteration, but not more than two, three or four alterations (e.g., substitutions, deletions, or insertions), or other CDR described herein.

The amino acids of an anti-Fn14 antibody or antigen-binding fragment thereof that interact with the Fn14 protein are preferably not mutated (or, if mutated, replaced by a conserved amino acid residue). In one embodiment of a variant of the P4A8 antibody or a variant of a P4A8-derived antibody or antigen-binding fragment (e.g., an antibody or antigen-binding fragment comprising SEQ ID NO:11 and SEQ ID NO:13), residue S32 of CDR L1 is not changed. In another embodiment, residue Y34 of CDR L1 is not changed. In another embodiment, residue Y36 of CDR L1 is not changed. In another embodiment, residue Y54 of CDR L2 is not changed. In another embodiment, residue R96 of CDR L3 is not changed. In another embodiment, residue D31 of CDR H1 is not changed. In another embodiment, residue Y32 of CDR H1 is not changed. In another embodiment, residue S52 of CDR H2 is not changed. In another embodiment, residue Y54 of CDR H2 is not changed. In another embodiment, residue N55 of CDR H2 is not changed. In another embodiment, residue Y57 of CDR H2 is not changed. In another embodiment, residue Y101 of CDR H3 is not changed. In another embodiment, residue Y105 of CDR H3 is not changed. In another embodiment, residue Y106 of CDR H3 is not changed.

In one embodiment, the antibody or antigen-binding fragment is as described herein with the proviso that at least 1, 2, 3, 4, 5 or 6 of the CDRs or 1 or 2 of the variable chains is not from a known antibody, e.g., ITEM-1, ITEM-2, ITEM-3 or ITEM-4.

In one embodiment, the antibody or antigen-binding fragment does not cross-react with other TNF and TNFR family members.

An antibody or antigen-binding fragment described herein can be, for example, a humanized antibody, a fully human antibody, a monoclonal antibody, a single chain antibody, a monovalent antibody, a polyclonal antibody, a chimeric antibody, a multispecific antibody (e.g., a bispecific antibody), a multivalent antibody, an F_{ab} fragment, an F_{(ab′)2} fragment, an F_{ab′} fragment, an F_{ab′} fragment, or an Fᵥ fragment.

An antibody or antigen-binding fragment described herein may be “multispecific,” e.g., bispecific, trispecific or of greater multispecificity, meaning that it recognizes and binds to two or more different epitopes present on one or more different antigens (e.g., proteins) at the same time. Thus, whether a binding molecule is “monospecfic” or “multispecific,” e.g., “bispecific,” refers to the number of different epitopes with which the binding molecule reacts. Multispecific antibodies may be specific for different epitopes of an Fn14 protein, or may be specific for Fn14 as well as for a heterologous epitope, such as a heterologous polypeptide or solid support material.

As used herein the term “valent” (as used in “multivalent antibody”) refers to the number of potential binding domains, e.g., antigen binding domains, present in a binding molecule. Each binding domain specifically binds one epitope. When a binding molecule comprises more than one binding domain, each binding domain may specifically bind the same epitope (for an antibody with two binding domains, termed “bivalent monospecific”) or to different epitopes (for an antibody with two binding domains, termed “bivalent bispecific”). An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Application Publication Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; WO 2007/109254; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992). These references are all incorporated by reference herein.

In certain embodiments, an anti-Fn14 antibody, e.g., one or the two heavy chains of the antibody, is linked to one or more scFv to form a bispecific antibody. In other embodiments, an anti-Fn14 antibody is in the form of an scFv that is linked to an antibody to form a bispecific molecule. Antibody-scFv constructs are described, e.g., in WO 2007/109254.

The heavy and light chains of the antibody can be substantially full-length. The protein can include at least one, and optionally two, complete heavy chains, and at least one, and optionally two, complete light chains or can include an antigen-binding fragment. In yet other embodiments, the antibody has a heavy chain constant region chosen from, e.g., IgG1, IgG2, IgG3, IgG4, IgM, IgA1, IgA2, IgD, and IgE. Typically, the heavy chain constant region is human or a modified form of a human constant region. In another embodiment, the antibody has a light chain constant region chosen from, e.g., kappa or lambda, particularly, kappa (e.g., human kappa).

In certain embodiments, the binding of antibodies or antigen binding fragments thereof results in cross-linking or clustering of the Fn14 receptor on the cell surface. For example, antibodies or antigen-binding fragments thereof may form a multimer, e.g., by binding to protein A, or may be multivalent.

An antibody or antigen-binding fragment described herein can be modified to enhance effector function, e.g., so as to enhance antigen-dependent cell-mediated cytotoxicity (ADCC) and/or complement dependent cytotoxicity (CDC) of the antibody or enhance cross-linking of the target receptor/Fn14. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989). In addition, an antibody can be defucosylated such that the modified antibody exhibits enhanced ADCC as compared to the non-defucosylated form of the antibody. See, e.g., WO2006089232.

Also provided herein are nucleic acids, e.g., DNAs, encoding an antibody or antigen binding fragment thereof, described herein. Nucleic acids that are at least about 80%, 85%, 90%, 95%, 97%, 98% or 99% identical to or hybridize under stringent hybridization conditions to these nucleic acids are also encompassed herein.

Also disclosed is an isolated cell that produces an antibody or antigen-binding fragment described herein. Also provided herein are cells, e.g., isolated cells, comprising a nucleic acid encoding a protein described herein. The cell can be, for example, a fused cell obtained by fusing a mammalian B cell and myeloma cell.

Also disclosed is a pharmaceutical composition comprising an antibody or antigen-binding fragment described herein and a pharmaceutically acceptable carrier.

In another aspect, the invention features a method of inducing death of a tumor cell, the method comprising contacting a tumor cell that expresses Fn14 with an amount of an antibody or antigen-binding fragment described herein effective to induce death of the tumor cell.

Also disclosed is a method of preventing or reducing tumor cell growth, the method comprising administering to a mammal having a tumor a pharmaceutical composition comprising an amount of an antibody or antigen-binding fragment described herein effective to prevent or reduce tumor cell growth.

Also disclosed is a method of treating a cancer, the method comprising administering to a mammal having a cancer a pharmaceutical composition comprising a therapeutically effective amount of an antibody or antigen-binding fragment described herein. The cancer can be, for example, a colon cancer or a breast cancer.

The mammal treated according to the methods described herein can be, e.g., a human, a mouse, a rat, a cow, a pig, a dog, a cat, or a monkey.

It should be understood that where reference is made herein to an “antibody or antigen-binding fragment,” this phrase may be replaced with “protein.” Accordingly, the description of the antibodies and antibody-binding fragments thereof also applies to proteins, such as proteins comprising these antibodies or antibody-binding fragments thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a graph showing that treatment of WiDr colon cancer cells in vitro with anti-Fn14 monoclonal antibodies P2D3, P4A8, P23G5, and P3D8 results in reduced cell viability, as measured by an MTT assay.

FIGS. 2A and 2B are a line graph (FIG. 2A) and a bar graph (FIG. 2B) showing that an anti-Fn14 monoclonal antibody (P4A8) can induce apoptosis of Widr colon cancer cells in vitro, as measured by a TUNEL assay.

FIG. 3 is a graph showing an example of Fn14+ breast tumor line (MDA-MB231) resistant to Fn14 monoclonal antibodies P2D3, P4A8, and P3G5 in vitro, as measured by an MTT assay.

FIG. 4 is a graph showing that Fn14 monoclonal antibodies P4A8, P2D3, P3G5, and P3D8 are agonists in an IL-8 induction assay, as measured by ng/ml IL-8 over various antibody concentrations.

FIG. 5 is a line graph (top) and bar graph (bottom) showing that anti-Fn14 monoclonal antibodies P2D3, P3G5, and P4A8 are efficacious in vivo to treat Widr cell colon tumors, as measured by tumor volume (mm³) on days post tumor inoculation (top) or by tumor weight (grams) on day 45.

FIG. 6 is a graph showing no obvious toxicities in animals treated with anti-Fn14 monoclonal antibodies P2D3, P3G5, and P4A8, as measured by weight (g) on days post tumor implantation.

FIG. 7 is a graph showing the efficacy of various doses and timings of dosing of anti-Fn14 monoclonal antibody P4A8 in treating large Widr tumors, as measured by tumor volume (mm³) on days post tumor inoculation.

FIG. 8 is a graph showing the dose response of Widr tumors to P4A8 anti-Fn14 monoclonal antibody, as measured by tumor volume (mm³) on days post tumor inoculation.

FIG. 9 is a graph showing the dose response of Widr tumors to P4A8 anti-Fn14 monoclonal antibody, as measured by percent test/control on days post tumor inoculation.

FIG. 10 is a graph showing no obvious toxicities in animals treated with various doses of anti-Fn14 monoclonal antibody P4A8, as measured by percent body weight change on days post tumor implantation.

FIG. 11 is a graph showing that anti-Fn14 monoclonal antibodies P2D3 and P4A8 are efficacious in vivo to treat MDA-MB231 breast cell tumors, as measured by tumor volume (mm³) on days post tumor inoculation.

FIG. 12 is a graph showing that anti-Fn14 monoclonal antibodies P4A8 and P2D3 are cross-reactive to Fn14 from multiple species (human (hu), murine (mu), and cynomolgus monkey (cyno).

FIG. 13 is a histogram showing that P4A8 binds significantly less well to human Fn14 having a W42A mutation relative to wildtype Fn14.

FIGS. 14A-14F are DNA sequences of the VH domain of the P4A8 antibody (FIG. 14A), the VH domain of the P3G5 antibody (FIG. 14B), the VH domain of the P2D3 antibody (FIG. 14C), the VL domain of the P4A8 antibody (FIG. 14D), the VL domain of the P3G5 antibody (FIG. 14E), and the VL domain of the P2D3 antibody (FIG. 14F).

FIG. 15 is a graph showing that hP4A8IgG1 and a multimeric version of hP4A8IgG1 kill WiDr colon cancer cells in vitro, as measured by an MTT assay.

FIG. 16 is a graph showing that the anti-Fn14 monoclonal antibodies P2D3, P3D8, P3G5 and P4A8 bind to human and cynomolgus monkey surface Fn14 with similar EC50 values.

FIG. 17 is a graph showing that the anti-Fn14 monoclonal antibodies P2D3, P3D8, P3G5 and P4A8 bind to murine surface Fn14 with similar EC50 values.

FIG. 18A and FIG. 18B are graphs showing that variants of huP4A8 with different heavy chain effector function bind to human (FIG. 18A) and rat (FIG. 18B) Fn14 with similar EC50 values.

FIG. 19A is a histogram showing that P4A8 binds to human, cynomolgus monkey, and mouse surface Fn14, but not Xenopus Fn14.

FIG. 19B is a histogram showing that the Fc-huTWEAK fusion protein binds to human, cynomolgus monkey, mouse and Xenopus surface Fn14.

FIG. 19C is a histogram showing that the muFc-muTWEAK fusion protein binds to human, cynomolgus monkey, mouse and Xenopus surface Fn14.

FIG. 20 is a gapped alignment of the Fn14 ectodomain.

FIG. 21A is a histogram showing Fc-TWEAK binds to all Fn14 W42A mutants.

FIG. 21B is a histogram showing that P4A8 binding to Fn14 is abrogated by mutation to W42A

FIG. 22 is a histogram showing that P4A8 binding to Fn14 is restored to normal when residue W42 is mutated to large hydrophobic residues W42F or W42Y.

FIG. 23A is a histogram showing Fc-TWEAK binding to a panel of human Fn14 point mutants.

FIG. 23B is a histogram showing P4A8 binding to a panel of human Fn14 point mutants.

FIG. 23C is a histogram showing P3G5 binding to a panel of human Fn14 point mutants.

FIG. 23D is a histogram showing P2D3 binding to a panel of human Fn14 point mutants.

FIG. 23E is a histogram showing ITEM-1 binding to a panel of human Fn14 point mutants.

FIG. 23F is a histogram showing ITEM-4 binding to a panel of human Fn14 point mutants.

FIG. 23G is a histogram showing ITEM-2 binding to a panel of human Fn14 point mutants.

FIG. 23H is a histogram showing ITEM-3 binding to a panel of human Fn14 point mutants.

FIG. 24 is a graph showing that different versions of huP4A8 are equivalent to chP4A8 as assayed by FACS dilution titration direct binding to surface human Fn14 transiently overexpressed in 293E cells.

FIG. 25 is a graph showing that different versions of huP4A8 retained Fn14 binding affinities essentially equivalent to chP4A8 assayed by competition ELISA.

FIG. 26 is a graph showing activation of Caspases 3/7 in WiDr cells in response to stimulation with hP4A8 and a multimeric version of hP4A8 (hP4A8-multi).

FIG. 27 is a graph showing induction of NFkB transcription factors in WiDr cells in response to P4A8.

FIG. 28 is a graph showing ADCC activity of hP4A8.IgG1 and Fc-crippled versions of P4A8 (hP4A8-IgG1agly and hP4A8.IgG4Pagly).

FIG. 29 is a graph showing the results of in vivo administration of P4A8 hIgG1 and P4A8hIgG4Pagly in the WiDr and MDA-MB231 assays.

FIG. 30, FIG. 31, and FIG. 32 are graphs showing the in vivo efficacy of the P4A8.hIgG1 antibody administered at various doses to WiDr human colon tumor-bearing athymic nude mice.

FIG. 33 and FIG. 34 are graphs showing the in vivo efficacy of the P4A8.hIgG1 antibody administered at various doses to MDA-MB-231 breast carcinoma tumor-bearing SCID mice.

FIG. 35 is a graph showing the efficacy of humanized P4A8 IgG1 in the Hs746T gastric carcinoma xenograft model.

FIGS. 36A and 36B are graphs showing the efficacy of humanized P4A8 IgG1 in the Hs746T (FIG. 36A) and N87 (FIG. 36B) gastric carcinoma xenograft models.

FIG. 37 is a graph showing the in vivo efficacy of the P4A8.hIgG1 antibody administered at various dosing schedules to WiDr human colon tumor-bearing athymic nude mice.

FIG. 38A is a graph depicting the ability of a panel of antibodies to crossblock binding of the antibody P2D3 to human Fn14.

FIG. 38B is a graph depicting the ability of a panel of antibodies to crossblock binding of the antibody P3G5 to human Fn14.

FIG. 38C is a graph depicting the ability of a panel of antibodies to crossblock binding of the antibody P4A8 to human Fn14.

FIG. 38D is a graph depicting the ability of a panel of antibodies to crossblock binding of the antibody ITEM-4 to human Fn14.

FIG. 38E is a graph depicting the ability of a panel of antibodies to crossblock binding of the antibody ITEM-3 to human Fn14.

### DETAILED DESCRIPTION

P4A8, P2D3, P3G5, and P3D8 are exemplary antibodies that specifically bind to human Fn14 and agonize Fn14 activity or mimic at least some of the activities resulting from binding of TWEAK to Fn14 on a cell surface. P2D3 and P3D8 have been found to have the same amino acid sequences. The anti-Fn14 antibodies described herein induce cell killing, e.g., by apoptosis, such as caspase-dependent apoptosis, and/or endogenous TNF-alpha mediated cell death, and can be used to treat or prevent diseases such as cancer, in which Fn14 is expressed.

### Fn14

Fn14 is an FGF-inducible receptor. It is often expressed at low levels on cells of normal tissues, and can be upregulated in injury or disease, or on cancer (e.g., tumor) cells. Without being bound by theory, it is believed that stimulation of Fn14 by an Fn14 ligand (e.g., TWEAK) can induce tumor cell death, and that an anti-Fn14 antibody will also be effective in killing tumor cells. It is also believed that Fn14 is overexpressed in human tumors. An anti-Fn14 antibody can trigger tumor cell death and therefore be therapeutically beneficial in treating cancer.

The sequence of human Fn14 is shown as:

(SEQ ID NO: 1)

MARGSLRRLLRLLVLGLWLALLRSVAGEQAPGTAPCSRGSSWSADLDKCM
DCASCRARPHSDFCLGCAAAPPAPFRLLWPILGGALSLTFVLGLLSGFLV
WRRCRRREKFTTPIEETGGEGCPAVALIQ.

Additional Fn14 protein sequences include: mouse Fn14 (e.g., NCBI accession no. AAF07882 or NP_{—}038777 or Q9CR75 or AAH25860), human Fn14 (e.g., NCBI accession no. NP_{—}057723 or BAA94792 or Q9NP84 or AAH02718 or AAF69108); rat Fn14 (e.g., NCBI accession no. NP_{—}851600 or AAH60537); and Xenopus Fn14 (e.g., NCBI accession no. AAR21225 or NP_{—}001083640). These Fn14 proteins can be used, e.g., as an immunogen to prepare anti-Fn14 antibodies. Anti-Fn14 antibodies can then be screened to identify agonist antibodies, as described herein.

### Anti-Fn14 Antibodies

This disclosure includes the sequences of specific examples of anti-Fn14 agonist antibodies, such as P4A8, P2D3, P3G5, and P3D8. Particular antibodies, such as these, can be made, for example, by preparing and expressing synthetic genes that encode the recited amino acid sequences or by mutating human germline genes to provide a gene that encodes the recited amino acid sequences. Moreover, these antibodies and other anti-Fn14 antibodies (e.g., agonist antibodies) can be produced, e.g., using one or more of the following methods.

Numerous methods are available for obtaining antibodies, particularly human antibodies. One exemplary method includes screening protein expression libraries, e.g., phage or ribosome display libraries. Phage display is described, for example, U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; and WO 90/02809. The display of Fab's on phage is described, e.g., in U.S. Pat. Nos. 5,658,727; 5,667,988; and 5,885,793.

In addition to the use of display libraries, other methods can be used to obtain a Fn14-binding antibody. For example, the Fn14 protein or a peptide thereof can be used as an antigen in a non-human animal, e.g., a rodent, e.g., a mouse, hamster, or rat. In addition, cells transfected with a cDNA encoding Fn14 can be injected into a non-human animal as a means of producing antibodies that effectively bind the cell surface Fn14 protein.

In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nature Genetics 7:13-21, U.S. 2003-0070185, WO 96/34096, and WO 96/33735.

In another embodiment, a monoclonal antibody is obtained from the non-human animal, and then modified, e.g., humanized or deimmunized. Winter describes an exemplary CDR-grafting method that may be used to prepare humanized antibodies described herein (U.S. Pat. No. 5,225,539). All or some of the CDRs of a particular human antibody may be replaced with at least a portion of a non-human antibody. It may only be necessary to replace the CDRs required for binding or binding determinants of such CDRs to arrive at a useful humanized antibody that binds to Fn14.

Humanized antibodies can be generated by replacing sequences of the Fv variable region that are not directly involved in antigen binding with equivalent sequences from human Fv variable regions. General methods for generating humanized antibodies are provided by Morrison, S. L. (1985) Science 229:1202-1207, by Oi et al. (1986) BioTechniques 4:214, and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al. (1992) J. Mol. Biol. 227:776-798; Cook, G. P. et al. (1995) Immunol. Today 16: 237-242; Chothia, D. et al. (1992) J. Mol. Bio. 227:799-817; and Tomlinson et al. (1995) EMBO J 14:4628-4638. The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

A non-human Fn14-binding antibody may also be modified by specific deletion of human T cell epitopes or “deimmunization” by the methods disclosed in WO 98/52976 and WO 00/34317. Briefly, the heavy and light chain variable regions of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions, or preferably, by single amino acid substitutions. As far as possible, conservative substitutions are made. Often, but not exclusively, an amino acid common to a position in human germline antibody sequences may be used. After the deimmunizing changes are identified, nucleic acids encoding V_{H} and V_{L} can be constructed by mutagenesis or other synthetic methods (e.g., de novo synthesis, cassette replacement, and so forth). A mutagenized variable sequence can, optionally, be fused to a human constant region, e.g., human IgG1 or kappa constant regions.

In some cases, a potential T cell epitope will include residues which are known or predicted to be important for antibody function. For example, potential T cell epitopes are usually biased towards the CDRs. In addition, potential T cell epitopes can occur in framework residues important for antibody structure and binding. Changes to eliminate these potential epitopes will in some cases require more scrutiny, e.g., by making and testing chains with and without the change. Where possible, potential T cell epitopes that overlap the CDRs can be eliminated by substitutions outside the CDRs. In some cases, an alteration within a CDR is the only option, and thus variants with and without this substitution can be tested. In other cases, the substitution required to remove a potential T cell epitope is at a residue position within the framework that might be critical for antibody binding. In these cases, variants with and without this substitution are tested. Thus, in some cases several variant deimmunized heavy and light chain variable regions are designed and various heavy/light chain combinations are tested to identify the optimal deimmunized antibody. The choice of the final deimmunized antibody can then be made by considering the binding affinity of the different variants in conjunction with the extent of deimmunization, particularly, the number of potential T cell epitopes remaining in the variable region. Deimmunization can be used to modify any antibody, e.g., an antibody that includes a non-human sequence, e.g., a synthetic antibody, a murine antibody other non-human monoclonal antibody, or an antibody isolated from a display library.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237. Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

### Affinity Maturation

In one embodiment, an anti-Fn14 antibody is modified, e.g., by mutagenesis, to provide a pool of modified antibodies. The modified antibodies are then evaluated to identify one or more antibodies which have altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used.

In some implementations, the mutagenesis is targeted to regions known or likely to be at the binding interface. If, for example, the identified binding proteins are antibodies, then mutagenesis can be directed to the CDR regions of the heavy or light chains as described herein. Further, mutagenesis can be directed to framework regions near or adjacent to the CDRs, e.g., framework regions, particularly within 10, 5, or 3 amino acids of a CDR junction. In the case of antibodies, mutagenesis can also be limited to one or a few of the CDRs, e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR region. For example, the germline CDR residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity, relative to the donor non-human antibody. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

Calculations of “sequence identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem. 22:259-306. Removal of any carbohydrate moieties present on the antibodies may be accomplished chemically or enzymatically as described in the art (Hakimuddin et al. (1987) Arch. Biochem. Biophys. 259:52; Edge et al. (1981) Anal. Biochem. 118:131; and Thotakura et al. (1987) Meth. Enzymol. 138:350). See, e.g., U.S. Pat. No. 5,869,046 for a modification that increases in vivo half life by providing a salvage receptor binding epitope.

In one embodiment, an antibody has CDR sequences that differ only insubstantially from those of P4A8, P2D3, P3G5, or P3D8. Insubstantial differences include minor amino acid changes, such as substitutions of 1 or 2 out of any of typically 5-7 amino acids in the sequence of a CDR, e.g., a Chothia or Kabat CDR. Typically an amino acid is substituted by a related amino acid having similar charge, hydrophobic, or stereochemical characteristics. Such substitutions would be within the ordinary skills of an artisan. Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al. (1991) J. Immun. 147:2657-62; Morgan et al. (1995) Immunology 86:319-24), or changing the species from which the constant region is derived.

The anti-Fn14 antibodies can be in the form of full length antibodies, or in the form of fragments of antibodies, e.g., Fab, F(ab′)₂, Fd, dAb, and scFv fragments. A fragment of an antibody can be an antigen-binding fragment, such as a variable region, e.g., VH or VL. Additional forms include a protein that includes a single variable domain, e.g., a camel or camelized domain. See, e.g., U.S. 2005-0079574 and Davies et al. (1996) Protein Eng. 9(6):531-7.

Provided herein are compositions comprising a mixture of anti-Fn14 antibody and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-Fn14 antibody comprising at least one deamidation site, wherein the pH of the composition is from about 5.0 to about 6.5, such that, e.g., at least about 90% of the anti-Fn14 antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g. as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e. the neutral amide side chain has been converted to a residue with an overall acidic character.

The term “mixture” as used herein in reference to a composition comprising an anti-Fn14 antibody, means the presence of both the desired anti-Fn14 antibody and one or more acidic variants thereof. The acidic variants may comprise predominantly deamidated anti-Fn14 antibody, with minor amounts of other acidic variant(s).

In one embodiment, an amino acid within the deamidation site (NG) or in the vicinity of the deamidation site is mutated to reduce or eliminate deamidation of the antibody. For example, CDR2 of the humanized P4A8 heavy chain SEQ ID NO:11 contains a deamidation site (NG) at positions 55 (N; Asn) and 56 (G; Gly). At least one amino acid substitution can be introduced within CDR2 of an antibody that contains CDR2 of SEQ ID NO:11 (or a variant thereof described herein) at a position corresponding to position 54, 55 or 56 of SEQ ID NO:11 so as to reduce or eliminate antibody deamidation, wherein: position 54 is Gly, Ala, Ser, Val, Thr, Leu, Ile, Met, Phe, Tyr, or Trp; position 55 is Asn, Gln, Arg, Asp, Ser, Gly, or Ala; position 56 is Gly, Ala, Ser, Val, Thr, Leu, Ile, Met, Phe, Tyr, or Trp; provided that when position 55 is Asn, position 56 is not Gly. For example, in the deamidation site NG, either the N or the G may be substituted for another amino acid. In one embodiment, the asparagine at amino acid position 55 (N55) is substituted with a serine (i.e., an N55S mutant of CDR2). Additional examples of analogs include: position 54 is Gly, position 55 is Asn, and position 56 is Val; position 54 is Gly, position 55 is Asn, and position 56 is Ala; position 54 is Gly, position 55 is Asp, and position 56 is Gly; position 54 is Gly, position 55 is Gln, and position 56 is Gly; position 54 is Gly, position 55 is Ala, and position 56 is Gly; position 54 is Gly, position 55 is Gly, and position 56 is Gly; position 54 is selected from the group consisting of Gly, Ala, Ser, Val, Thr, Leu, Ile, Met, Phe, Tyr, and Trp, position 55 is Ala, and position 56 is Gly; and position 54 is selected from the group consisting of Gly, Ala, Ser, Val, Thr, Leu, Ile, Met, Phe, Tyr, and Trp, position 55 is Gly, and position 56 is Gly (see, e.g., WO2003/073982).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

In certain embodiments, an anti-Fn14 antibody inhibits angiogenesis. Anti-Fn14 antibodies may alternatively stimulate angiogenesis or have no effect on angiogenesis. An effect on angiogenesis may be determined in in vitro or in vivo assays, e.g., in an endothelial proliferation assays on HUVEC cells, or in a corneal pocket assay, wound closure assays and other assays, known in the art.

### Antibody Fragments

Traditionally, antibody fragments were derived via proteolytic digestion of intact antibodies. Alternatively, these fragments can be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. In other embodiments, the antibody of choice is a single chain Fv fragment (scFv). Fv and scFv contain intact combining sites that are devoid of constant regions; thus, they are suitable for reduced nonspecific binding during in vivo use. scFv fusion proteins may be constructed to yield fusion of an effector protein at either the amino or the carboxy terminus of an scFv. The antibody fragment may also be a “linear antibody,” e.g., as described in U.S. Pat. No. 5,641,870. Such linear antibody fragments may be monospecific or bispecific.

### Bispecific Antibodies

Bispecific antibodies are antibodies that have binding specificities for at least two different epitopes. Exemplary bispecific antibodies may bind to two different epitopes of the Fn14 protein. Other such antibodies may combine an Fn14 binding site with a binding site for another protein. Alternatively, an anti-Fn14 arm may be combined with an arm which binds to a triggering molecule on a leukocyte such as a T-cell receptor molecule (e.g., CD3), or Fc receptors for IgG (Fc-gamma-R), such as Fc-gamma-RI (CD64), Fc-gamma-RII (CD32) and Fc-gamma-RIII (CD16), so as to focus and localize cellular defense mechanisms to the Fn14-expressing cell. Bispecific antibodies may also be used to localize cytotoxic agents to cells which express Fn14. These antibodies possess an Fn14-binding arm and an arm that binds the cytotoxic agent (e.g., saporin, anti-interferon-alpha, vinca alkaloid, ricin A chain, methotrexate, or a radioactive isotope hapten). Bispecific antibodies can be prepared as full length antibodies or antibody fragments (e.g., F(ab′)₂ bispecific antibodies).

Traditional production of full length bispecific antibodies is based on the co-expression of two immunoglobulin heavy chain-light chain pairs, where the two chains have different specificities (Millstein et al., Nature 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites.

### Multivalent Antibodies

A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Antibody Production

Some antibodies, e.g., Fab's, can be produced in bacterial cells, e.g., E. coli cells. Antibodies can also be produced in eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one preferred embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, and a cell from a transgenic animal, e.g., a transgenic mammal. For example, the cell is a mammary epithelial cell.

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced.

In an exemplary system for antibody expression, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G coupled matrix.

For antibodies that include an Fc domain, the antibody production system preferably synthesizes antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof (1992) Adv. Immunol. 51:1-84; Jefferis et al. (1998) Immunol. Rev. 163:59-76). In one embodiment, the Fc domain is produced in a mammalian expression system that appropriately glycosylates the residue corresponding to asparagine 297. The Fc domain or other region of the antibody can also include other eukaryotic post-translational modifications.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

### Characterization

The binding properties of an antibody may be measured by any standard method, e.g., one of the following methods: BIACORE™ analysis, Enzyme Linked Immunosorbent Assay (ELISA), Fluorescence Resonance Energy Transfer (FRET), x-ray crystallography, sequence analysis and scanning mutagenesis. Preferably, the antibody has a statistically significant effect that indicates that the antibody promotes one or more activities of Fn14 (e.g., promotes Fn14 signaling).

### Surface Plasmon Resonance (SPR)

The binding interaction of a protein of interest and a target (e.g., Fn14) can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Information from SPR can be used to provide an accurate and quantitative measure of the equilibrium dissociation constant (K_{d}), and kinetic parameters, including Kₒₙ and K_{off}, for the binding of a biomolecule to a target.

Epitopes can also be directly mapped by assessing the ability of different antibodies to compete with each other for binding to Fn14 (e.g., human Fn14) using BIAcore chromatographic techniques (Pharmacia BIAtechnology Handbook, “Epitope Mapping”, Section 6.3.2, (May 1994); see also Johne et al. (1993) J. Immunol. Methods, 160:191-198). Additional general guidance for evaluating antibodies, e.g., in Western blots and immunoprecipitation assays, can be found in Antibodies: A Laboratory Manual, ed. by Harlow and Lane, Cold Spring Harbor press (1988)).

### Agonist Antibodies

Once antibodies that bind to Fn14 have been identified, the antibodies can be assayed to determine if the antibodies are agonists of Fn14. Anti-Fn14 antibodies can be evaluated for their ability to increase or activate a downstream effect of Fn14 signaling (e.g., increase or activate events downstream of Fn14 engagement by a natural ligand (e.g., TWEAK)) or to mimic an effect caused by the binding of a natural ligand (e.g., TWEAK) to Fn14. The mimicking can be the same degree or to a lesser or greater degree than the effect of natural ligand, as long as the same type of effect is caused.

For example, an antibody can be evaluated for the ability to induce or enhance cell killing of Fn-14 expressing cells (e.g., cancer cells such as WiDr colon cancer cells). In another embodiment, an antibody is evaluated for the ability to induce or enhance IL-8 secretion in Fn-14 expressing cells (e.g., A375 cells), induces or increases NF-KB p52 and/or cell cycle inhibitor p21 Waf1/Cip1 expression or protein levels.

Antibodies having activities that are similar to those of mouse or humanized P4A8, e.g., wherein the same amount of antibody produces an effect that is at least about 50%, 75%, 80%, 90%, 95%, 97%, 98% or 99% the effect produced by the mouse or humanized P4A8, may be used for treating cancer as described herein. For example, an anti-Fn14 antibody that induces the production of an amount of IL-8 that is at least about 50% of that produced by P4A8; an antibody that induces cell killing at least 50% as efficacious as P4A8; and an antibody that induces NK-KB p52 or p21 expression to amounts that are at least about 50% of those produced by P4A8, respectively, can be used for treating cancer. Of course, antibodies having activities that are stronger than those of P4A8 or other antibodies described herein are also encompassed herein.

### Deposits

Hybridomas producing the monoclonal antibody 1.P4A8.3C7 (P4A8), the monoclonal antibody 1.P3G5.1E4 (P3G5), and the monoclonal antibody 1.P2D3.3D5 (P2D3) have been deposited with the American Type Culture Collection (ATCC) under the terms of the Budapest Treaty on the International Recognition of the Deposit of Microorganisms for the Purpose of Patent Procedure on Apr. 7, 2009, and bear the accession numbers PTA-9947 (P4A8), PTA-9949 (P3G5), and PTA-9948 (P2D3). Applicants acknowledge their duty to replace the deposits should the depository be unable to furnish a sample when requested due to the condition of the deposit before the end of the term of a patent issued hereon. Applicants also acknowledge their responsibility to notify the ATCC of the issuance of such a patent, at which time the deposit will be made available to the public. Prior to that time, the deposit will be made available to the Commissioner of Patents under the terms of 37 C.F.R. § 1.14 and 35 U.S.C. § 112.

Antibodies with Reduced Effector Function

The interaction of antibodies and antibody-antigen complexes with cells of the immune system triggers a variety of responses, referred to herein as effector functions. IgG antibodies activate effector pathways of the immune system by binding to members of the family of cell surface Fcγ receptors and to C1q of the complement system. Ligation of effector proteins by clustered antibodies triggers a variety of responses, including release of inflammatory cytokines, regulation of antigen production, endocytosis, and cell killing. In some clinical applications these responses are crucial for the efficacy of a monoclonal antibody. In others they provoke unwanted side effects such as inflammation and the elimination of antigen-bearing cells. Accordingly, the present invention further relates to Fn14-binding proteins, including antibodies, with altered, e.g., reduced, effector functions.

Effector function of an anti-Fn14 antibody of the present invention may be determined using one of many known assays. The anti-Fn14 antibody's effector function may be reduced relative to a second anti-Fn14 antibody. In some embodiments, the second anti-Fn14 antibody may be any antibody that binds Fn14 specifically. In other embodiments, the second Fn14-specific antibody may be any of the antibodies of the invention, such as P4A8. In other embodiments, where the anti-Fn14 antibody of interest has been modified to reduce effector function, the second anti-Fn14 antibody may be the unmodified or parental version of the antibody.

Exemplary effector functions include Fc receptor binding, phagocytosis, apoptosis, pro-inflammatory responses, down-regulation of cell surface receptors (e.g. B cell receptor; BCR), etc. Other effector functions include antibody-dependent cell-mediated cytotoxicity (ADCC), whereby antibodies bind Fc receptors on cytotoxic T cells, natural killer (NK) cells, or macrophages leading to cell death, and complement-dependent cytotoxicity (CDC), which is cell death induced via activation of the complement cascade (reviewed in Daeron, Annu. Rev. Immunol. 15:203-234 (1997); Ward and Ghetie, Therapeutic Immunol. 2:77-94 (1995); and Ravetch and Kinet, Annu. Rev. Immunol. 9:457-492 (1991)). Such effector functions generally require the Fc region to be combined with a binding domain (e.g. an antibody variable domain) and can be assessed using standard assays that are known in the art (see, e.g., WO 05/018572, WO 05/003175, and U.S. Pat. No. 6,242,195).

Effector functions can be avoided by using antibody fragments lacking the Fc domain such as Fab, Fab′2, or single chain Fv. An alternative has been to use the IgG4 subtype antibody, which binds to FcγRI but which binds poorly to C1q and FcγRII and RIII. The IgG2 subtype also has reduced binding to Fc receptors, but retains significant binding to the H131 allotype of FcγRIIa and to C1q. Thus, additional changes in the Fc sequence are required to eliminate binding to all the Fc receptors and to C1q.

Several antibody effector functions, including ADCC, are mediated by Fc receptors (FcRs), which bind the Fc region of an antibody. The affinity of an antibody for a particular FcR, and hence the effector activity mediated by the antibody, may be modulated by altering the amino acid sequence and/or post-translational modifications of the Fc and/or constant region of the antibody.

FcRs are defined by their specificity for immunoglobulin isotypes; Fc receptors for IgG antibodies are referred to as FcγR, for IgE as FCεR, for IgA as FcαR and so on. Three subclasses of FcγR have been identified: FcγRI (CD64), FcγRII (CD32) and FcγRIII (CD16). Both FcγRII and FcγRIII have two types: FcγRIIA (CD32) and FcγRIIB (CD32); and FcγRIIIA (CD16a) and FcγRIIIB (CD16b). Because each FcγR subclass is encoded by two or three genes, and alternative RNA splicing leads to multiple transcripts, a broad diversity in FcγR isoforms exists. For example, FcγRII (CD32) includes the isoforms IIa, IIb1, IIb2 IIb3, and IIc.

The binding site on human and murine antibodies for FcγR has been previously mapped to the so-called “lower hinge region” consisting of residues 233-239 (EU index numbering as in Kabat et al., Sequences of Proteins of Immunological Interest, 5th Ed. Public Health Service, National Institutes of Health, Bethesda, Md. (1991), Woof et al. Molec. Immunol. 23:319-330 (1986); Duncan et al. Nature 332:563 (1988); Canfield and Morrison, J. Exp. Med. 173:1483-1491 (1991); Chappel et al., Proc. Natl. Acad. Sci. USA 88:9036-9040 (1991)). Of residues 233-239, P238 and S239 are among those cited as possibly being involved in binding. Other previously cited areas possibly involved in binding to FcγR are: G316-K338 (human IgG) for human FcγRI (by sequence comparison only; no substitution mutants were evaluated) (Woof et al. Molec Immunol. 23:319-330 (1986)); K274-R301 (human IgG1) for human FcγRIII (based on peptides) (Sarmay et al. Molec. Immunol. 21:43-51 (1984)); and Y407-R416 (human IgG) for human FcγRIII (based on peptides) (Gergely et al. Biochem. Soc. Trans. 12:739-743 (1984) and Shields et al. J Biol Chem 276: 6591-6604 (2001), Lazar G A et al. Proc Natl Acad Sci 103: 4005-4010 (2006). These and other stretches or regions of amino acid residues involved in FcR binding may be evident to the skilled artisan from an examination of the crystal structures of Ig-FcR complexes (see, e.g., Sondermann et al. 2000 Nature 406(6793):267-73 and Sondermann et al. 2002 Biochem Soc Trans. 30(4):481-6). Accordingly, the anti-Fn14 antibodies of the present invention include modifications of one or more of the aforementioned residues.

Other known approaches for reducing mAb effector function include mutating amino acids on the surface of the mAb that are involved in effector binding interactions (Lund, J., et al. (1991) J. Immunol. 147(8): 2657-62; Shields, R. L. et al. (2001) J. Biol. Chem. 276(9): 6591-604; and using combinations of different subtype sequence segments (e.g., IgG2 and IgG4 combinations) to give a greater reduction in binding to Fcγ receptors than either subtype alone (Armour et al., Eur. J. Immunol. (1999) 29: 2613-1624; Mol. Immunol. 40 (2003) 585-593). For example, sites of N-linked glycosylation can be removed as a means of reducing effector function.

A large number of Fc variants having altered and/or reduced affinities for some or all Fc receptor subtypes (and thus for effector functions) are known in the art. See, e.g., US 2007/0224188; US 2007/0148171; US 2007/0048300; US 2007/0041966; US 2007/0009523; US 2007/0036799; US 2006/0275283; US 2006/0235208; US 2006/0193856; US 2006/0160996; US 2006/0134105; US 2006/0024298; US 2005/0244403; US 2005/0233382; US 2005/0215768; US 2005/0118174; US 2005/0054832; US 2004/0228856; US 2004/132101; US 2003/158389; see also U.S. Pat. Nos. 7,183,387; 6,737,056; 6,538,124; 6,528,624; 6,194,551; 5,624,821; 5,648,260.

In CDC, the antibody-antigen complex binds complement, resulting in the activation of the complement cascade and generation of the membrane attack complex. Activation of the classical complement pathway is initiated by the binding of the first component of the complement system (C1q) to antibodies (of the appropriate subclass) which are bound to their cognate antigen; thus the activation of the complement cascade is regulated in part by the binding affinity of the immunoglobulin to C1q protein. To activate the complement cascade, it is necessary for C1q to bind to at least two molecules of IgG1, IgG2, or IgG3, but only one molecule of IgM, attached to the antigenic target (Ward and Ghetie, Therapeutic Immunology 2:77-94 (1995) p. 80). To assess complement activation, a CDC assay, e.g. as described in Gazzano-Santoro et al., J. Immunol. Methods 202:163 (1996), may be performed.

It has been proposed that various residues of the IgG molecule are involved in binding to C1q including the Glu318, Lys320 and Lys322 residues on the CH2 domain, amino acid residue 331 located on a turn in close proximity to the same beta strand, the Lys235 and Gly237 residues located in the lower hinge region, and residues 231 to 238 located in the N-terminal region of the CH2 domain (see e.g., Xu et al., J. Immunol. 150:152A (Abstract) (1993), WO94/29351; Tao et al, J. Exp. Med., 178:661-667 (1993); Brekke et al., Eur. J. Immunol., 24:2542-47 (1994); Burton et al; Nature, 288:338-344 (1980); Duncan and Winter, Nature 332:738-40 (1988); Idusogie et al J Immunol 164: 4178-4184 (2000; U.S. Pat. No. 5,648,260, and U.S. Pat. No. 5,624,821). As an example in IgG1, two mutations in the COOH terminal region of the CH2 domain of human IgG1—K322A and P329A—do not activate the CDC pathway and were shown to result in more than a 100 fold decrease in C1q binding (U.S. Pat. No. 6,242,195).

Thus, in certain embodiments of the invention, one or more of these residues may be modified, substituted, or removed or one or more amino acid residues may be inserted so as to decrease CDC activity of the Fn14 antibodies provided herein. For example in some embodiments, it may be desirable to reduce or eliminate effector function(s) of the subject antibodies in order to reduce or eliminate the potential of further activating immune responses. Antibodies with decreased effector function may also reduce the risk of thromboembolic events in subjects receiving the antibodies.

In certain other embodiments, the present invention provides an anti-Fn14 antibody that exhibits reduced binding to one or more FcR receptors but that maintains its ability to bind complement (e.g., to a similar or, in some embodiments, to a lesser extent than a native, non-variant, or parent anti-Fn14 antibody). Accordingly, an anti-Fn14 antibody of the present invention may bind and activate complement while exhibiting reduced binding to an FcR, such as, for example, FcγRIIa (e.g., FcγRIIa expressed on platelets). Such an antibody with reduced or no binding to FcγRIIa (such as FcγRIIa expressed on platelets, for example) but that can bind C1q and activate the complement cascade to at least some degree will reduce the risk of thromboembolic events while maintaining perhaps desirable effector functions. In alternative embodiments, an anti-Fn14 antibody of the present invention exhibits reduced binding to one or more FcRs but maintains its ability to bind one or more other FcRs. See, for example, US 2007-0009523, 2006-0194290, 2005-0233382, 2004-0228856, and 2004-0191244, which describe various amino acid modifications that generate antibodies with reduced binding to FcRI, FcRII, and/or FcRIII, as well as amino acid substitutions that result in increased binding to one FcR but decreased binding to another FcR.

Accordingly, effector functions involving the constant region of an anti-Fn14 antibody may be modulated by altering properties of the constant region, and the Fc region in particular. In certain embodiments, the anti-Fn14 antibody having reduced effector function is compared with a second antibody with effector function and which may be a non-variant, native, or parent antibody comprising a native constant or Fc region that mediates effector function. In particular embodiments, effector function modulation includes situations in which an activity is abolished or completely absent.

A native sequence Fc or constant region comprises an amino acid sequence identical to the amino acid sequence of a Fc or constant chain region found in nature. Preferably, a control molecule used to assess relative effector function comprises the same type/subtype Fc region as does the test or variant antibody. A variant or altered Fc or constant region comprises an amino acid sequence which differs from that of a native sequence heavy chain region by virtue of at least one amino acid modification (such as, for example, post-translational modification, amino acid substitution, insertion, or deletion). Accordingly, the variant constant region may contain one or more amino acid substitutions, deletions, or insertions that results in altered post-translational modifications, including, for example, an altered glycosylation pattern. A parent antibody or Fc region is, for example, a variant having normal effector function used to construct a constant region (i.e., Fc) having altered, e.g., reduced, effector function.

Antibodies with altered (e.g., reduced or eliminated) effector function(s) may be generated by engineering or producing antibodies with variant constant, Fc, or heavy chain regions. Recombinant DNA technology and/or cell culture and expression conditions may be used to produce antibodies with altered function and/or activity. For example, recombinant DNA technology may be used to engineer one or more amino acid substitutions, deletions, or insertions in regions (such as, for example, Fc or constant regions) that affect antibody function including effector functions. Alternatively, changes in post-translational modifications, such as, e.g. glycosylation patterns (see below), may be achieved by manipulating the host cell and cell culture and expression conditions by which the antibody is produced.

Amino acid alterations, such as amino acid substitutions, can alter the effector function of the anti-Fn14 antibodies of the present invention without affecting antigen binding affinity. The amino acid substitutions described above (e.g., Glu318, Kys320, Lys332, Lys235, Gly237, K332, and P329), for example, may be used to generate antibodies with reduced effector function.

In other embodiments, amino acid substitutions may be made for one or more of the following amino acid residues: 234, 235, 236, 237, 297, 318, 320, and 322 of the heavy chain constant region (see U.S. Pat. No. 5,624,821 and U.S. Pat. No. 5,648,260). Such substitutions may alter effector function while retaining antigen binding activity. An alteration at one or more of amino acids 234, 235, 236, and 237 can decrease the binding affinity of the Fc region for FcγRI receptor as compared to an unmodified or non-variant antibody. Amino acid residues 234, 236, and/or 237 may be substituted with alanine, for example, and amino acid residue 235 may be substituted with glutamine, for example. In another embodiment, an anti-Fn14 IgG1 antibody may comprise a substitution of Leu at position 234 with Ala, a substitution of Leu at position 235 with Glu, and a substitution of Gly at position 237 with Ala.

Additionally or alternatively, the Fc amino acid residues at 318, 320, and 322 may be altered. These amino acid residues, which are highly conserved in mouse and human IgGs, mediate complement binding. It has been shown that alteration of these amino acid residues reduces C1q binding but does not alter antigen binding, protein A binding, or the ability of the Fc to bind to mouse macrophages.

In another embodiment, an anti-Fn14 antibody of the present invention is an IgG4 immunoglobulin comprising substitutions that reduce or eliminate effector function. The IgG4 Fc portion of an anti-Fn14 antibody of the invention may comprise one or more of the following substitutions: substitution of proline for glutamate at residue 233, alanine or valine for phenylalanine at residue 234 and alanine or glutamate for leucine at residue 235 (EU numbering, Kabat, E. A. et al. (1991), supra). Further, removing the N-linked glycosylation site in the IgG4 Fc region by substituting Ala for Asn at residue 297 (EU numbering) may further reduce effector function and eliminate any residual effector activity that may exist. Another exemplary IgG4 mutant with reduced effector function is the IgG4 subtype variant containing the mutations S228P and L235E (PE mutation) in the heavy chain constant region. This mutation results in reduced effector function. See U.S. Pat. No. 5,624,821 and U.S. Pat. No. 5,648,260. Another exemplary mutation in the IgG4 context that reduces effector function is S228P/T229A, as described herein.

Other exemplary amino acid sequence changes in the constant region include but are not limited to the Ala-Ala mutation described by Bluestone et al. (see WO 94/28027 and WO 98/47531; also see Xu et al. 2000 Cell Immunol 200; 16-26). Thus in certain embodiments, anti-Fn14 antibodies with mutations within the constant region including the Ala-Ala mutation may be used to reduce or abolish effector function. According to these embodiments, the constant region of an anti-Fn14 antibody comprises a mutation to an alanine at position 234 or a mutation to an alanine at position 235. Additionally, the constant region may contain a double mutation: a mutation to an alanine at position 234 and a second mutation to an alanine at position 235.

In one embodiment, an anti-Fn14 antibody comprises an IgG4 framework, wherein the Ala-Ala mutation would describe a mutation(s) from phenylalanine to alanine at position 234 and/or a mutation from leucine to alanine at position 235. In another embodiment, the anti-Fn14 antibody comprises an IgG1 framework, wherein the Ala-Ala mutation would describe a mutation(s) from leucine to alanine at position 234 and/or a mutation from leucine to alanine at position 235. An anti-Fn14 antibody may alternatively or additionally carry other mutations, including the point mutation K322A in the CH2 domain (Hezareh et al. 2001 J. Virol. 75: 12161-8).

Other exemplary amino acid substitutions are provided in WO 94/29351 (which is incorporated herein by reference in its entirety), which recites antibodies having mutations in the N-terminal region of the CH2 domain that alter the ability of the antibodies to bind to FcRI, thereby decreasing the ability of antibodies to bind to C1q which in turn decreases the ability of the antibodies to fix complement. Also see Cole et al. (J. Immunol. (1997) 159: 3613-3621), which describes mutations in the upper CH2 regions in IgG2 that result in lower FcR binding.

Methods of generating any of the aforementioned antibody variants comprising amino acid substitutions are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody.

Site-directed mutagenesis is well known in the art (see, e.g., Carter et al. Nucleic Acids Res. 13:4431-4443 (1985) and Kunkel et al., Proc. Natl. Acad. Sci. USA 82:488 (1987)).

PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene 34:315-323 (1985).

Another embodiment of the present invention relates to an anti-Fn14 antibody with reduced effector function in which the antibody's Fc region, or portions thereof, is swapped with an Fc region (or with portions thereof) having naturally reduced effector inducing activity. For example, human IgG4 constant region exhibits reduced or no complement activation. Further, the different IgG molecules differ in their binding affinity for FcR, which may be due at least in part to the varying length and flexibility of the IgGs' hinge regions (which decreases in the order IgG3>IgG1>IgG4>IgG2). For example, IgG4 exhibits reduced or no binding to FcγRIIa. For examples of chimeric molecules and chimeric constant regions, see, e.g., Gillies et al. (Cancer Res. 1999, 59: 2159-2166) and Mueller et al. (Mol. Immunol. 1997, 34: 441-452).

The invention also relates to anti-Fn14 antibodies with reduced effector function in which the Fc region is completely absent. Such antibodies may also be referred to as antibody derivatives and antigen-binding fragments of the present invention. Such derivatives and fragments may be fused to non-antibody protein sequences or non-protein structures, especially structures designed to facilitate delivery and/or bioavailability when administered to an animal, e.g., a human subject (see below).

As discussed above, changes within the hinge region also affect effector functions. For example, deletion of the hinge region may reduce affinity for Fc receptors and may reduce complement activation (Klein et al. 1981 PNAS USA 78: 524-528). The present disclosure therefore also relates to antibodies with alterations in the hinge region.

In particular embodiments, antibodies of the present invention may be modified to inhibit complement dependent cytotoxicity (CDC). Modulated CDC activity may be achieved by introducing one or more amino acid substitutions, insertions, or deletions in an Fc region of the antibody (see, e.g., U.S. Pat. No. 6,194,551 and U.S. Pat. No. 6,242,195). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO 99/51642, Duncan & Winter Nature 322: 738-40 (1988); U.S. Pat. No. 5,648,260; U.S. Pat. No. 5,624,821; and WO 94/29351.

It is further understood that effector function may vary according to the binding affinity of the antibody. For example, antibodies with high affinity may be more efficient in activating the complement system compared to antibodies with relatively lower affinity (Marzocchi-Machado et al. 1999 Immunol Invest 28: 89-101). Accordingly, an antibody may be altered such that the binding affinity for its antigen is reduced (e.g., by changing the variable regions of the antibody by methods such as substitution, addition, or deletion of one or more amino acid residues). An antibody with reduced binding affinity may exhibit reduced effector functions, including, for example, reduced ADCC and/or CDC.

Anti-Fn14 antibodies of the present invention with reduced effector function include antibodies with reduced binding affinity for one or more Fc receptors (FcRs) relative to a parent or non-variant anti-Fn14 antibody. Accordingly, anti-Fn14 antibodies with reduced FcR binding affinity includes anti-Fn14 antibodies that exhibit a 1.5-fold, 2-fold, 2.5-fold, 3-fold, 4-fold, or 5-fold or higher decrease in binding affinity to one or more Fc receptors compared to a parent or non-variant anti-Fn14 antibody. In some embodiments, an anti-Fn14 antibody with reduced effector function binds to an FcR with about 10-fold less affinity relative to a parent or non-variant antibody. In other embodiments, an anti-Fn14 antibody with reduced effector function binds to an FcR with about 15-fold less affinity or with about 20-fold less affinity relative to a parent or non-variant antibody. The FcR receptor may be one or more of FcγRI (CD64), FcγRII (CD32), and FcγRIII, and isoforms thereof, and FcεR, FcμR, FcδR, and/or an FcaR. In particular embodiments, an anti-Fn14 antibody with reduced effector function exhibits a 1.5-fold, 2-fold, 2.5-fold, 3-fold, 4-fold, or 5-fold or higher decrease in binding affinity to FcγRIIa.

Accordingly, in certain embodiments, an anti-Fn14 antibody of the present invention exhibits reduced binding to a complement protein relative to a second anti-Fn14 antibody. In certain embodiments, an anti-Fn14 antibody of the invention exhibits reduced binding by a factor of about 1.5-fold or more, about 2-fold or more, about 3-fold or more, about 4-fold or more, about 5-fold or more, about 6-fold or more, about 7-fold or more, about 8-fold or more, about 9-fold or more, about 10-fold or more, or about 15-fold or more, relative to a second anti-Fn14 antibody.

Certain embodiments of the present invention relate to an anti-Fn14 antibody comprising one or more heavy chain CDR sequences selected from CDR-H1 of SEQ ID NO:2, CDR-H2 of SEQ ID NO:2 and CDR-H3 of SEQ ID NO:2, wherein the antibody further comprises a variant Fc region that confers reduced effector function compared to a native or parental Fc region. In further embodiments, the anti-Fn14 antibody comprises at least two of the CDRs, and in other embodiments the antibody comprises all three of the heavy chain CDR sequences.

Other embodiments of the present invention relate to an anti-Fn14 antibody comprising one or more light chain CDR sequences selected from CDR-L1 of SEQ ID NO:5, CDR-L2 of SEQ ID NO:5 and CDR-L3 of SEQ ID NO:5, the antibody further comprising a variant Fc region that confers reduced effector function compared to a native or parental Fc region. In further embodiments, the anti-Fn14 antibody comprises at least two of the light chain CDRs, and in other embodiments the antibody comprises all three of the light chain CDR sequences.

In further embodiments of the present invention, the anti-Fn14 antibody with reduced effector function comprises all three light chain CDR sequences of SEQ ID NO:5 and comprises all three heavy chain CDR sequences of SEQ ID NO:2.

In other embodiments, the invention relates to an anti-Fn14 antibody comprising a V_{L} sequence of amino acids 1-111 of SEQ ID NO:9, the antibody further comprising a variant Fc region that confers reduced effector function compared to a native or parental Fc region.

In other embodiments, the invention relates to an anti-Fn14 antibody comprising a V_{H} sequence of amino acids 1-121 of SEQ ID NO:8, the antibody further comprising a variant Fc region that confers reduced effector function compared to a native or parental Fc region.

Anti-Fn14 Antibodies with Altered Glycosylation

Glycan removal produces a structural change that should greatly reduce binding to all members of the Fc receptor family across species. In glycosylated antibodies, including anti-Fn14 antibodies, the glycans (oligosaccharides) attached to the conserved N-linked site in the CH2 domains of the Fc dimer are enclosed between the CH2 domains, with the sugar residues making contact with specific amino acid residues on the opposing CH2 domain. Different glycosylation patterns are associated with different biological properties of antibodies (Jefferis and Lund, 1997, Chem. Immunol., 65: 111-128; Wright and Morrison, 1997, Trends Biotechnol., 15: 26-32). Certain specific glycoforms confer potentially advantageous biological properties. Loss of the glycans changes spacing between the domains and increases their mobility relative to each other and is expected to have an inhibitory effect on the binding of all members of the Fc receptor family. For example, in vitro studies with various glycosylated antibodies have demonstrated that removal of the CH2 glycans alters the Fc structure such that antibody binding to Fc receptors and the complement protein C1Q are greatly reduced. Another known approach to reducing effector functions is to inhibit production of or remove the N-linked glycans at position 297 (EU numbering) in the CH2 domain of the Fc (Nose et al., 1983 PNAS 80: 6632; Leatherbarrow et al., 1985 Mol. Immunol. 22: 407; Tao et al., 1989 J. Immunol. 143: 2595; Lund et al., 1990 Mol. Immunol. 27: 1145; Dorai et al., 1991 Hybridoma 10:211; Hand et al., 1992 Cancer Immunol. Immunother. 35:165; Leader et al., 1991 Immunology 72: 481; Pound et al., 1993 Mol. Immunol. 30:233; Boyd et al., 1995 Mol. Immunol. 32: 1311). It is also known that different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, phagocytosis and antibody feedback, in addition to effector functions of the antibody (e.g., binding to the complement complex C1, which induces CDC, and binding to FcγR receptors, which are responsible for modulating the ADCC pathway) (Nose and Wigzell, 1983; Leatherbarrow and Dwek, 1983; Leatherbarrow et al., 1985; Walker et al., 1989; Carter et al., 1992, PNAS, 89: 4285-4289).

Accordingly, another means of modulating effector function of antibodies includes altering glycosylation of the antibody constant region. Altered glycosylation includes, for example, a decrease or increase in the number of glycosylated residues, a change in the pattern or location of glycosylated residues, as well as a change in sugar structure(s). The oligosaccharides found on human IgGs affects their degree of effector function (Raju, T. S. BioProcess International April 2003. 44-53); the microheterogeneity of human IgG oligosaccharides can affect biological functions such as CDC and ADCC, binding to various Fc receptors, and binding to C1q protein (Wright A. & Morrison S L. TIBTECH 1997, 15 26-32; Shields et al. J Biol Chem. 2001 276(9):6591-604; Shields et al. J Biol Chem. 2002; 277(30):26733-40; Shinkawa et al. J Biol Chem. 2003 278(5):3466-73; Umana et al. Nat Biotechnol. 1999 February; 17(2): 176-80). For example, the ability of IgG to bind C1q and activate the complement cascade may depend on the presence, absence or modification of the carbohydrate moiety positioned between the two CH2 domains (which is normally anchored at Asn297) (Ward and Ghetie, Therapeutic Immunology 2:77-94 (1995).

Glycosylation sites in an Fc-containing polypeptide, for example an antibody such as an IgG antibody, may be identified by standard techniques. The identification of the glycosylation site can be experimental or based on sequence analysis or modeling data. Consensus motifs, that is, the amino acid sequence recognized by various glycosyl transferases, have been described. For example, the consensus motif for an N-linked glycosylation motif is frequently NXT or NXS, where X can be any amino acid except proline. Several algorithms for locating a potential glycosylation motif have also been described. Accordingly, to identify potential glycosylation sites within an antibody or Fc-containing fragment, the sequence of the antibody is examined, for example, by using publicly available databases such as the website provided by the Center for Biological Sequence Analysis (see NetNGlyc services for predicting N-linked glycosylation sites and NetOGlyc services for predicting O-linked glycosylation sites).

In vivo studies have confirmed the reduction in the effector function of aglycosyl antibodies. For example, an aglycosyl anti-CD8 antibody is incapable of depleting CD8-bearing cells in mice (Isaacs, 1992 J. Immunol. 148: 3062) and an aglycosyl anti-CD3 antibody does not induce cytokine release syndrome in mice or humans (Boyd, 1995 supra; Friend, 1999 Transplantation 68:1632).

Importantly, while removal of the glycans in the CH2 domain appears to have a significant effect on effector function, other functional and physical properties of the antibody remain unaltered. Specifically, it has been shown that removal of the glycans had little to no effect on serum half-life and binding to antigen (Nose, 1983 supra; Tao, 1989 supra; Dorai, 1991 supra; Hand, 1992 supra; Hobbs, 1992 Mol. Immunol. 29:949).

Although there is in vivo validation of the aglycosyl approach, there are reports of residual effector function with aglycosyl mAbs (see, e.g., Pound, J. D. et al. (1993) Mol. Immunol. 30(3): 233-41; Dorai, H. et al. (1991) Hybridoma 10(2): 211-7). Armour et al. show residual binding to FcγRIIa and FcγRIIb proteins (Eur. J. Immunol. (1999) 29: 2613-1624; Mol. Immunol. 40 (2003) 585-593). Thus a further decrease in effector function, particularly complement activation, may be important to guarantee complete ablation of activity in some instances. For that reason, aglycosyl forms of IgG2 and IgG4 and a G1/G4 hybrid are envisioned as being useful in methods and antibody compositions of the invention having reduced effector functions.

The anti-Fn14 antibodies of the present invention may be modified or altered to elicit reduced effector function(s) (compared to a second Fn14-specific antibody) while optionally retaining the other valuable attributes of the Fc portion.

Accordingly, in certain embodiments, the present invention relates to aglycosyl anti-Fn14 antibodies with decreased effector function, which are characterized by a modification at the conserved N-linked site in the CH2 domains of the Fc portion of the antibody. A modification of the conserved N-linked site in the CH2 domains of the Fc dimer can lead to aglycosyl anti-Fn14 antibodies. Examples of such modifications include mutation of the conserved N-linked site in the CH2 domains of the Fc dimer, removal of glycans attached to the N-linked site in the CH2 domains, and prevention of glycosylation. For example, an aglycosyl anti-Fn14 antibody may be created by changing the canonical N-linked Asn site in the heavy chain CH2 domain to a Gln residue (see, for example, WO 05/03175 and US 2006-0193856).

In one embodiment of present invention, the modification comprises a mutation at the heavy chain glycosylation site to prevent glycosylation at the site. Thus, in one embodiment of this invention, the aglycosyl anti-Fn14 antibodies are prepared by mutation of the heavy chain glycosylation site, i.e., mutation of N298Q (N297 using Kabat EU numbering) and expressed in an appropriate host cell. For example, this mutation may be accomplished by following the manufacturer's recommended protocol for unique site mutagenesis kit from Amersham-Pharmacia Biotech® (Piscataway, N.J., USA).

The mutated antibody can be stably expressed in a host cell (e.g. NSO or CHO cell) and then purified. As one example, purification can be carried out using Protein A and gel filtration chromatography. It will be apparent to those of skill in the art that additional methods of expression and purification may also be used.

In another embodiment of the present invention, the aglycosyl anti-Fn14 antibodies have decreased effector function, wherein the modification at the conserved N-linked site in the CH2 domains of the Fc portion of said antibody or antibody derivative comprises the removal of the CH2 domain glycans, i.e., deglycosylation. These aglycosyl anti-Fn14 antibodies may be generated by conventional methods and then deglycosylated enzymatically. Methods for enzymatic deglycosylation of antibodies are well known to those of skill in the art (Williams, 1973; Winkelhake & Nicolson, 1976 J. Biol. Chem. 251:1074-80.).

In another embodiment of this invention, deglycosylation may be achieved by growing host cells which produce the antibodies in culture medium comprising a glycosylation inhibitor such as tunicamycin (Nose & Wigzell, 1983). That is, the modification is the reduction or prevention of glycosylation at the conserved N-linked site in the CH2 domains of the Fc portion of said antibody.

In other embodiments of this invention, recombinant X polypeptides (or cells or cell membranes containing such polypeptides) may be used as an antigen to generate an anti-Fn14 antibody or antibody derivatives, which may then be deglycosylated.

In alternative embodiments, agyclosyl anti-Fn14 antibodies or anti-Fn14 antibodies with reduced glycosylation of the present invention, may be produced by the method described in Taylor et al. (WO 05/18572 and US 2007-0048300). For example, in one embodiment, an anti-Fn14 aglycosyl antibody may be produced by altering a first amino acid residue (e.g., by substitution, insertion, deletion, or by chemical modification), wherein the altered first amino acid residue inhibits the glycosylation of a second residue by either steric hindrance or charge or both. In certain embodiments, the first amino acid residue is modified by amino acid substitution. In further embodiments, the amino acid substitution is selected from the group consisting of Gly, Ala, Val, Leu, Ile, Phe, Asn, Gln, Trp, Pro, Ser, Thr, Tyr, Cys, Met, Asp, Glu, Lys, Arg, and His. In other embodiments, the amino acid substitution is a non-traditional amino acid residue. The second amino acid residue may be near or within a glycosylation motif, for example, an N-linked glycosylation motif that contains the amino acid sequence NXT or NXS. In one exemplary embodiment, the first amino acid residue is amino acid 299 and the second amino acid residue is amino acid 297, according to the Kabat numbering. For example, the first amino acid substitution may be T299A, T299N, T299G, T299Y, T299C, T299H, T299E, T299D, T299K, T299R, T299G, T2991, T299L, T299M, T299F, T299P, T299W, and T299V, according to the Kabat numbering. In particular embodiments, the amino acid substitution is T299C.

Effector function may also be reduced by modifying an antibody of the present invention such that the antibody contains a blocking moiety. Exemplary blocking moieties include moieties of sufficient steric bulk and/or charge such that reduced glycosylation occurs, for example, by blocking the ability of a glycosidase to glycosylate the polypeptide. The blocking moiety may additionally or alternatively reduce effector function, for example, by inhibiting the ability of the Fc region to bind a receptor or complement protein. In some embodiments, the present invention relates to an Fn14-binding protein, e.g., an anti-Fn14 antibody, comprising a variant Fc region, the variant Fc region comprising a first amino acid residue and an N-glycosylation site, the first amino acid residue modified with side chain chemistry to achieve increased steric bulk or increased electrostatic charge compared to the unmodified first amino acid residue, thereby reducing the level of or otherwise altering glycosylation at the N-glycosylation site. In certain of these embodiments, the variant Fc region confers reduced effector function compared to a control, non-variant Fc region. In further embodiments, the side chain with increased steric bulk is a side chain of an amino acid residue selected from the group consisting of Phe, Trp, His, Glu, Gln, Arg, Lys, Met and Tyr. In yet further embodiments, the side chain chemistry with increased electrostatic charge is a side chain of an amino acid residue selected from the group consisting of Asp, Glu, Lys, Arg, and His.

Accordingly, in one embodiment, glycosylation and Fc binding can be modulated by substituting T299 with a charged side chain chemistry such as D, E, K, or R. The resulting antibody will have reduced glycosylation as well as reduced Fc binding affinity to an Fc receptor due to unfavorable electrostatic interactions.

In another embodiment, a T299C variant antibody, which is both aglycosylated and capable of forming a cysteine adduct, may exhibit less effector function (e.g., FcγRI binding) compared to its aglycosylated antibody counterpart (see, e.g., WO 05/18572). Accordingly, alteration of a first amino acid proximal to a glycosylation motif can inhibit the glycosylation of the antibody at a second amino acid residue; when the first amino acid is a cysteine residue, the antibody may exhibit even further reduced effector function. In addition, inhibition of glycosylation of an antibody of the IgG4 subtype may have a more profound affect on FcγRI binding compared to the effects of agycosylation in the other subtypes.

In additional embodiments, the present invention relates to anti-Fn14 antibodies with altered glycosylation that exhibit reduced binding to one or more FcR receptors and that optionally also exhibit increased or normal binding to one or more Fc receptors and/or complement—e.g., antibodies with altered glycosylation that at least maintain the same or similar binding affinity to one or more Fc receptors and/or complement as a native, control anti-Fn14 antibody). For example, anti-Fn14 antibodies with predominantly Man₅GlcNAc₂N-glycan as the glycan structure present (e.g., wherein Man₅GlcNAc₂N-glycan structure is present at a level that is at least about 5 mole percent more than the next predominant glycan structure of the Ig composition) may exhibit altered effector function compared to an anti-Fn14 antibody population wherein Man₅GlcNAc₂N-glycan structure is not predominant. Antibodies with predominantly this glycan structure exhibit decreased binding to FcγRIIa and FcγRIIb, increased binding to FcγRIIIa and FcγRIIIb, and increased binding to C1q subunit of the C1 complex (see US 2006-0257399). This glycan structure, when it is the predominant glycan structure, confers increased ADCC, increased CDC, increased serum half-life, increased antibody production of B cells, and decreased phagocytosis by macrophages.

In general, the glycosylation structures on a glycoprotein will vary depending upon the expression host and culturing conditions (Raju, T S. BioProcess International April 2003. 44-53). Such differences can lead to changes in both effector function and pharmacokinetics (Israel et al. Immunology. 1996; 89(4):573-578; Newkirk et al. P. Clin. Exp. 1996; 106(2):259-64). For example, galactosylation can vary with cell culture conditions, which may render some immunoglobulin compositions immunogenic depending on their specific galactose pattern (Patel et al., 1992. Biochem J. 285: 839-845). The oligosaccharide structures of glycoproteins produced by non-human mammalian cells tend to be more closely related to those of human glycoproteins. Further, protein expression host systems may be engineered or selected to express a predominant Ig glycoform or alternatively may naturally produce glycoproteins having predominant glycan structures. Examples of engineered protein expression host systems producing a glycoprotein having a predominant glycoform include gene knockouts/mutations (Shields et al., 2002, JBC, 277: 26733-26740); genetic engineering in (Umana et al., 1999, Nature Biotech., 17: 176-180) or a combination of both. Alternatively, certain cells naturally express a predominant glycoform—for example, chickens, humans and cows (Raju et al., 2000, Glycobiology, 10: 477-486). Thus, the expression of an anti-Fn14 antibody or antibody composition having altered glycosylation (e.g., predominantly one specific glycan structure) can be obtained by one skilled in the art by selecting at least one of many expression host systems. Protein expression host systems that may be used to produce anti-Fn14 antibodies of the present invention include animal, plant, insect, bacterial cells and the like. For example, US 2007-0065909, 2007-0020725, and 2005-0170464 describe producing aglycosylated immunoglobulin molecules in bacterial cells. As a further example, Wright and Morrison produced antibodies in a CHO cell line deficient in glycosylation (1994 J Exp Med 180: 1087-1096) and showed that antibodies produced in this cell line were incapable of complement-mediated cytolysis. Other examples of expression host systems found in the art for production of glycoproteins include: CHO cells: Raju WO 99/22764 and Presta WO 03/35835; hybridoma cells: Trebak et al., 1999, J. Immunol. Methods, 230: 59-70; insect cells: Hsu et al., 1997, JBC, 272:9062-970, and plant cells: Gerngross et al., WO 04/74499. To the extent that a given cell or extract has resulted in the glycosylation of a given motif, art recognized techniques for determining if the motif has been glycosylated are available, for example, using gel electrophoresis and/or mass spectroscopy.

Additional methods for altering glycosylation sites of antibodies are described, e.g., in U.S. Pat. No. 6,350,861 and U.S. Pat. No. 5,714,350, WO 05/18572 and WO 05/03175; these methods can be used to produce anti-Fn14 antibodies of the present invention with altered, reduced, or no glycosylation.

The aglycosyl anti-Fn14 antibodies with reduced effector function may be antibodies that comprise modifications or that may be conjugated to comprise a functional moiety. Such moieties include a blocking moiety (e.g., a PEG moiety, cysteine adducts, etc.), a detectable moiety (e.g., fluorescent moieties, radioisotopic moieties, radiopaque moieties, etc., including diagnostic moieties), a therapeutic moiety (e.g., cytotoxic agents, anti-inflammatory agents, immunomodulatory agents, anti-infective agents, anti-cancer agents, anti-neurodegenerative agents, radionuclides, etc.), and/or a binding moiety or bait (e.g., that allows the antibody to be pre-targeted to a tumor and then to bind a second molecule, composed of the complementary binding moiety or prey and a detectable moiety or therapeutic moeity, as described above).

### Fn14-Associated Disorders

An anti-Fn14 antibody (such as an antibody described herein) can be used to treat a variety of disorders, such as an Fn14-associated disorder. For example, the antibody can be used to treat cancer, e.g., solid tumor cancers, in a patient. Examples of cancers that can be treated with an anti-Fn14 antibody include colon cancer and breast cancer. Still other examples of cancers that can be treated include: Anal, Bile duct, Bladder, Bone, secondary Bone, Bowel (colon & rectum; colorectal cancer), Brain, secondary Brain, Breast, secondary Breast, Cervix, Pediatric cancers, Endocrine, Eye, Gall bladder, Gastrointestinal (e.g., Gastric), Gullet (esophagus), Head & neck, Kaposi's sarcoma, Kidney, Larynx, Leukemia, acute lymphoblastic Leukemia, acute myeloid Leukemia, chronic lymphocytic Leukemia, chronic myeloid Leukemia, Liver, secondary Liver, Lung (e.g., NSCLC), secondary Lung, secondary Lymph nodes, Lymphoma, Hodgkin Lymphoma, non-Hodgkin Lymphoma, Melanoma, Mesothelioma, Myeloma, Neuroendocrine, Ovary, Esophageal, Pancreas (pancreatic cancer), Penis, Prostate, Rectal, Skin, Soft tissue sarcomas, Spinal cord, Stomach, Testes, Thymus, Thyroid, Unknown primary, Vagina, Vulva, and Womb (uterus; endometrial cancer).

Tumors that can be treated include those having Fn14 expression, e.g., high Fn14 expression, relative to the Fn14 expression level on a normal adult cell.

The term “treating” refers to administering a composition described herein in an amount, manner, and/or mode effective to improve a condition, symptom, or parameter associated with a disorder or to prevent progression or exacerbation of the disorder (including secondary damage caused by the disorder) to either a statistically significant degree or to a degree detectable to one skilled in the art.

In some embodiments, treatment of a patient that has a solid tumor with an anti-Fn14 antibody described herein results in a reduction of the size of the solid tumor by at least 10%, at least 20%, at least 30%, at least 40%, at least 50%, at least 60%, at least 70%, at least 80%, or at least 90%.

A subject who is at risk for, diagnosed with, or who has one of these disorders can be administered an anti-Fn14 antibody in an amount and for a time to provide an overall therapeutic effect. The anti-Fn14 antibody can be administered alone (monotherapy) or in combination with other agents (combination therapy). In the case of a combination therapy, the amounts and times of administration can be those that provide, e.g., an additive or a synergistic therapeutic effect. Further, the administration of the anti-Fn14 antibody (with or without the second agent) can be used as a primary, e.g., first line treatment, or as a secondary treatment, e.g., for subjects who have an inadequate response to a previously administered therapy (i.e., a therapy other than one with an anti-Fn14 antibody). In some embodiments, an anti-Fn14 antibody can be used in combination with another chemotherapeutic agent. In some embodiments, the combination therapy includes the use of two or more anti-Fn14 antibodies, e.g., at least one of the anti-Fn14 antibodies described herein in combination with another anti-Fn14 antibody, e.g., two or more of the anti-Fn14 antibodies described herein.

In certain embodiments, a subject receiving an anti-Fn14 antibody has Fn14 expression on tumor cells, e.g., high Fn14 expression relative to the level of expression of Fn14 on normal adult cells. In certain embodiments, a subject receiving an anti-Fn14 antibody is not a subject having no detectable Fn14 level on the surface of its tumor cells. The level of Fn14 on tumor cells may be measured by immunohistochemistry or FACS using, e.g., an antibody described herein.

In certain embodiments of combination therapies, the therapy or treatment with which the anti-Fn14 antibody therapy is combined does not significantly induce expression of Fn14 on normal cells, such as to minimize unwanted potential toxicity effects. In certain embodiments of combination therapies, in which the second therapy or treatment induces Fn14 levels on normal cells, the an anti-Fn14 antibody is administered after administration of the first therapy or treatment of the combination therapy, at a time when any increase in Fn14 levels have essentially returned to normal.

In certain embodiments, a subject that is treated with an Fn14 antibody described herein, e.g., an Fn14 agonist antibody, is not a subject who has a disease that is or may be exacerbated by an Fn14 agonist antibody. For example, in certain embodiments, a subject that is treated with an Fn14 antibody, e.g., an agonist antibody, is not a subject having an autoimmune disease, rheumatoid arthritis, multiple sclerosis, stroke, fibrosis, a neurodegenerative disease, Alzheimer's disease, ALS, systemic lupus erythematosus, or a disease set forth in U.S. Pat. No. 7,169,387, WO 03/086311, WO2006/088890 or WO 2006/089095. In certain embodiments, a subject receiving an anti-Fn14 antibody is not a subject having or likely to develop an inflammatory or autoimmune disease, e.g., rheumatoid arthritis, intestinal bowel disease, lupus, Crohn's disease, multiple sclerosis, diabetes, psoriasis, acute graft versus host disease (GVHD), pancreatitis, delayed type hypersensitivity (DTH).

In certain embodiments, a subject receiving an anti-Fn14 antibody has received or receives or will receive an anti-inflammatory treatment. For example, a subject may be treated with an anti-inflammatory agent at the same time, before and/or after treatment with an anti-Fn14 Ab. Exemplary anti-inflammatory agents include methotrexate, a TNF-alpha blocking agent, a Tweak blocking agent, a disease modifying anti-rheumatic drug (DMARD), non-steroidal anti-inflammatory drugs such as salicylates (Aspirin), a gold compound, Hydroxychloroquine, penicillamine, steroids, and immunosuppressive drugs.

In certain embodiments, a method comprises determining the level of Fn14 expressed on tumor cells of a subject, and then, if the level is higher than that on normal cells, e.g., normal cells of the same type or lineage as the cancer cells, treating the subject with an anti-Fn14 antibody, and if the level is lower than that on normal cells, e.g., normal cells of the same type or lineage as the cancer cells or if there is no detectable level of Fn14, not treating the subject with an anti-Fn14 antibody.

In certain embodiments, a method comprises determining whether Fn14 is expressed (at a minimum threshold level) on tumor cells of a subject, and then, if Fn14 expression is detected (at the minimum threshold level), treating the subject with an anti-Fn14 antibody, and if Fn14 expression is not detected (at the minimum threshold level), not treating the subject with an anti-Fn14 antibody.

In some embodiments, an Fn14 antibody may be useful in treating a disease in which Fn14 expression is not detected.

### Cancer

An anti-Fn14 antibody can be used to treat a subject diagnosed as having or as being at risk for cancer, e.g., colon cancer or breast cancer. The cancer can be primary, secondary or metastatic.

Therapy: An anti-Fn14 antibody (such as an antibody described herein) can be used to treat cancer or reduce the risk of cancer occurrence, alone or in combination with another cancer therapy, such as a standard of care therapy. In addition to the combination treatments described herein, an anti-Fn14 antibody can be used in combination with Gemcitabine (e.g., for the treatment of pancreatic cancer), taxol or trastuzumab (e.g., for the treatment of breast cancer), Irinotecan, bevacizumab, 5-fluorouracil, or cetuximab (e.g., for the treatment of colon cancer), or trastuzumab (e.g., for the treatment of gastric cancer).

Other cancer treatments include surgery, chemotherapy, radiation therapy, immunotherapy, and monoclonal antibody therapy. An Fn14 antibody can be used in combination with any of these treatment modalities. The choice of therapy depends upon the location and grade of the tumor and the stage of the disease, as well as the general state of the patient.

Complete removal of the cancer without damage to the rest of the body is the goal of treatment. Sometimes this can be accomplished by surgery, but the propensity of cancers to invade adjacent tissue or to spread to distant sites by microscopic metastasis often limits its effectiveness. The effectiveness of chemotherapy is often limited by toxicity to other tissues in the body. Radiation can also cause damage to normal tissue.

Surgery: In theory, cancers can be cured if entirely removed by surgery, but this is not always possible. When the cancer has metastasized to other sites in the body prior to surgery, complete surgical excision is usually impossible. In one model of cancer progression, tumors grow locally, then spread to the lymph nodes, then to the rest of the body. This has given rise to the popularity of local-only treatments such as surgery for small cancers. Even small localized tumors are increasingly recognized as possessing metastatic potential.

Examples of surgical procedures for cancer include mastectomy for breast cancer and prostatectomy for prostate cancer. The goal of the surgery can be either the removal of only the tumor, or the entire organ. A single cancer cell is invisible to the naked eye but can re-grow into a new tumor.

In addition to removal of the primary tumor, surgery is often necessary for staging, e.g., determining the extent of the disease and whether it has metastasized to regional lymph nodes. Staging is a major determinant of prognosis and of the need for adjuvant therapy.

Occasionally, surgery is necessary for palliative treatment, to control symptoms such as spinal cord compression or bowel obstruction.

An anti-Fn14 antibody can be used in combination with surgery, before, during, and/or after surgery. E.g., the antibody can be administered locally at the site of surgery, e.g., on the tissue in and/or surrounding the area from which a tumor was excised, or as therapy after a patient who has undergone surgery is recovering.

Radiation therapy: Radiation therapy (also called radiotherapy, X-ray therapy, or irradiation) is the use of ionizing radiation to kill cancer cells and shrink tumors. Radiation therapy can be administered externally via external beam radiotherapy (EBRT) or internally via brachytherapy. The effects of radiation therapy are localized and confined to the region being treated. Radiation therapy injures or destroys cells in the area being treated (the “target tissue”). The goal of radiation therapy is to damage as many cancer cells as possible, while limiting harm to nearby healthy tissue, Hence, it is given in many fractions, allowing healthy tissue to recover between fractions.

Radiation therapy may be used to treat almost every type of solid tumor, including cancers of the brain, breast, cervix, larynx, lung, pancreas, prostate, skin, stomach, uterus, or soft tissue sarcomas. Radiation is also used to treat leukemia and lymphoma. Radiation dose to each site depends on a number of factors, including the radiosensitivity of each cancer type and whether there are tissues and organs nearby that may be damaged by radiation.

An anti-Fn14 antibody can be used in combination with radiation therapy e.g., before, during, and/or after radiation therapy. E.g., the antibody can be administered locally at a site that was/is being/will be irradiated.

Chemotherapy: Chemotherapy is the treatment of cancer with drugs that can destroy cancer cells. “Chemotherapy” usually refers to cytotoxic drugs which affect rapidly dividing cells in general, in contrast with targeted therapy. Chemotherapy drugs interfere with cell division in various possible ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can.

Examples of chemotherapeutic agents used in cancer therapy include: Amsacrine, Bleomycin, Busulfan, Capecitabine, Carboplatin, Carmustine, Chlorambucil, Cisplatin, Cladribine, Clofarabine, Crisantaspase, Cyclophosphamide, Cytarabine, Dacarbazine, Dactinomycin, Daunorubicin, Docetaxel, Doxorubicin, Epirubicin, Etoposide, Fludarabine, 5 Fluorouracil (5FU), Gemcitabine, Gliadel implants, Hydroxycarbamide, Idarubicin, Ifosfamide, Irinotecan, Leucovorin, Liposomal doxorubicin, Liposomal daunorubicin, Lomustine, Melphalan, Mercaptopurine, Mesna, Methotrexate, Mitomycin, Mitoxantrone, Oxaliplatin, Paclitaxel, Pemetrexed, Pentostatin, Procarbazine, Raltitrexed, Streptozocin, Tegafur-uracil, Temozolomide, Teniposide, Thiotepa, Tioguanine, Topotecan, Treosulfan, Vinblastine, Vincristine, Vindesine, and Vinorelbine.

Because some drugs work better together than alone, two or more drugs are often given at the same time. Often, two or more chemotherapy agents are used as a combination chemotherapy. An anti-Fn14 antibody can be used in combination with chemotherapy (e.g., with one or more chemotherapeutics), e.g., before, during, or after the use of the chemotherapeutic agent(s).

Targeted therapies: Targeted therapy constitutes the use of agents specific for the deregulated proteins or other identified molecules of cancer cells. Small molecule targeted therapy drugs are generally inhibitors of enzymatic domains on mutated, overexpressed, or otherwise critical proteins within the cancer cell. Prominent examples are the tyrosine kinase inhibitors imatinib and gefitinib. Monoclonal antibody therapy is another strategy in which the therapeutic agent is an antibody which specifically binds to aprotein on the surface of the cancer cells. Examples include anti-Fn14 antibodies, the anti-HER2/neu antibody trastuzumab (HERCEPTIN®) typically used in breast cancer, and the anti-CD20 antibody rituximab, typically used in a variety of B-cell malignancies.

Targeted therapy can also involve small peptides as “homing devices” which can bind to cell surface receptors or affected extracellular matrix surrounding the tumor. Radionuclides which are attached to this peptides (e.g., RGDs) eventually kill the cancer cell if the nuclide decays in the vicinity of the cell.

An anti-Fn14 antibody can be used in combination with another targeted therapy, e.g., a targeted therapy described herein, e.g., before, during, or after the use of the targeted therapy.

Photodynamic therapy: Photodynamic therapy (PDT) is a ternary treatment for cancer involving a photosensitizer, tissue oxygen, and light (often using lasers). PDT can be used as treatment, e.g., for basal cell carcinoma (BCC) or lung cancer; PDT can also be useful in removing traces of malignant tissue after surgical removal of large tumors.

An anti-Fn14 antibody can be used in combination with photodynamic therapy, e.g., before, during, or after the use of the photodynamic therapy.

Immunotherapy: Cancer immunotherapy refers to a diverse set of therapeutic strategies designed to induce the patient's own immune system to fight the tumor. Contemporary methods for generating an immune response against tumors include intravesical BCG immunotherapy for superficial bladder cancer, and use of interferons (e.g., interferon-gamma) and other cytokines to induce an immune response, e.g., in renal cell carcinoma and melanoma patients.

Allogeneic hematopoietic stem cell transplantation can be considered a form of immunotherapy, since the donor's immune cells will often attack the tumor in a graft-versus-tumor effect.

An anti-Fn14 antibody can be used in combination with an immunotherapy described herein, e.g., before, during, or after the use of the other immunotherapy.

Hormonal therapy: The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial.

An anti-Fn14 antibody can be used in combination with a hormonal therapy described herein, e.g., before, during, or after the use of the hormonal therapy.

Colon Cancer. Colon cancer is cancer that starts in the large intestine (colon) or the rectum (end of the colon). Such cancer is sometimes referred to as “colorectal cancer.” The most common type is colon carcinoma. Other types of colon cancer such as lymphoma, carcinoid tumors, melanoma, and sarcomas are rare.

Causes: According to the American Cancer Society, colorectal cancer is one of the leading causes of cancer-related deaths in the United States. There is no single cause for colon cancer. N early all colon cancers begin as benign polyps, which slowly develop into cancer. A higher risk for colon cancer exists if a patient has: colorectal polyps, cancer elsewhere in the body, a family history of colon cancer, ulcerative colitis, Crohn's disease, personal history of breast cancer, and/or certain genetic syndromes also increase the risk of developing colon cancer.

Symptoms: Many cases of colon cancer have no symptoms. The following symptoms, however, may indicate colon cancer: diarrhea, constipation, or other change in bowel habits, blood in the stool, unexplained anemia, abdominal pain and tenderness in the lower abdomen, intestinal obstruction, weight loss with no known reason, and narrow stools. With proper screening, colon cancer can be detected before the development of symptoms, when it is most curable.

Exams and Tests: The physical exam rarely shows any problems, although an abdominal mass may be felt. A rectal exam may reveal a mass in patients with rectal cancer, but not colon cancer. Imaging tests to diagnose colorectal cancer include: colonoscopy and sigmoidoscopy. A fecal occult blood test (FOBT) may detect small amounts of blood in the stool, which could suggest colon cancer. However, this test is often negative in patients with colon cancer. For this reason, a FOBT is typically performed along with colonoscopy or sigmoidoscopy. A complete blood count may reveal show signs of anemia with low iron levels.

If a patient has colorectal cancer, additional tests, staging, will be done to see if the cancer has spread: Stage 0: Very early cancer on the innermost layer of the intestine; stage I: cancer is in the inner layers of the colon; stage II: cancer has spread through the muscle wall of the colon; stage III: cancer has spread to the lymph nodes; stage IV: cancer that has spread to other organs.

Treatment: Treatment depends partly on the stage of the cancer. In general, treatments may include: chemotherapy medicines to kill cancer cells, surgery to remove cancer cells, and/or radiation therapy to destroy cancerous tissue. Further, an anti-Fn14 antibody described herein can be used to treat colon cancer, alone or in combination with another treatment described herein. Stage 0 colon cancer may be treated by removing the cancer cells, often during a colonoscopy. Further, an anti-Fn14 antibody described herein can be used to treat stage 0 colon cancer, alone or in combination with another treatment described herein (e.g., surgery or chemotherapy). For stages I, II, and III cancer, more extensive surgery is needed to remove the part of the colon that is cancerous. Also, an anti-Fn14 antibody described herein can be used to treat stage I, II, or III colon cancer, alone or in combination with another treatment described herein (e.g., surgery, chemotherapy, or radiotherapy). Almost all patients with stage III colon cancer should receive chemotherapy after surgery for approximately 6-8 months. 5-fluorouracil is an example of a chemotherapeutic used to treat stage III colon cancer. Chemotherapy is also used to treat patients with stage IV colon cancer. Irinotecan, oxaliplatin, and 5-fluorouracil are the three most commonly used drugs. Capecitabine is also used. Further, an anti-Fn14 antibody described herein can be used to treat stage IV colon cancer, alone or in combination with another treatment described herein (e.g., surgery, chemotherapy, or radiotherapy). For patients with stage IV disease that has spread to the liver, various treatments directed specifically at the liver can be used. This may include cutting out the cancer, ablation, or cryotherapy. Chemotherapy or radiation can sometimes be delivered directly into the liver. Further, an anti-Fn14 antibody described herein can be used to treat colon cancer that has metastasized to the liver or other location in the body alone or in combination with another treatment described herein (e.g., surgery, chemotherapy, or radiotherapy). While radiation therapy is occasionally used in patients with colon cancer, it is usually used in combination with chemotherapy for patients with stage III rectal cancer. Similarly, an anti-Fn14 antibody described herein can be used to treat stage IV colon cancer, e.g., in combination with radiation therapy.

Prognosis: How well a patient does depends on many things, including the stage of the cancer. In general, when treated at an early stage, more than 90% of patients survive at least 5 years after their diagnosis. However, only about 39% of colorectal cancer is found at an early stage. The 5-year survival rate drops considerably once the cancer has spread. If the patient's colon cancer does not recur within 5 years, it is considered cured. Stage I, II, and III cancers are considered potentially curable. In most cases, stage IV cancer is not curable.

Possible Complications: Complications include metastasis, recurrence of carcinoma within the colon, development of a second primary colorectal cancer.

Prevention: Colon cancer can almost always be caught in its earliest and most curable stages by colonoscopy. Almost all men and women age 50 and older should have a colonoscopy. Dietary and lifestyle modifications are important. Some evidence suggests that low-fat and high-fiber diets may reduce your risk of colon cancer. An anti-Fn-14 antibody can be used to reduce the risk of or prevent the development of colon cancer, e.g., in a patient identified as being at risk for colon cancer.

Breast Cancer. Breast cancer is a cancer that starts in the tissues of the breast. The two main types of breast cancer are ductal carcinoma and lobular carcinoma. In rare cases, breast cancer can start in other areas of the breast. Many breast cancers are estrogen-sensitive (estrogen receptor positive cancer or ER positive cancer). Some breast cancers are HER2-positive.

Causes: Risk factors include:

Age and gender—Risk of developing breast cancer increases with age. The majority of advanced breast cancer cases are found in women over age 50. Women are 100 times more likely to get breast cancer then men.

Family history of breast cancer—A higher risk for breast cancer exists if a close relative has had breast, uterine, ovarian, or colon cancer. About 20-30% of women with breast cancer have a family history of the disease.

Genetics—The most common gene defects are found in the BRCA1 and BRCA2 genes. Women with mutations in one of these genes have up to an 80% chance of getting breast cancer sometime during their life. Other genetic defects have been linked to breast cancer, including those found in the ATM gene, the CHEK-2 gene, and the p53 tumor suppressor gene, but these are rare.

Menstrual cycle—Women who get their periods early (before age 12) or went through menopause late (after age 55) have an increased risk for breast cancer.

Alcohol use—Drinking more than 1-2 glasses of alcohol a day may increase the risk for breast cancer.

Childbirth—Women who have never had children or who had them only after age 30 have an increased risk for breast cancer. Being pregnant more than once or becoming pregnant at an early age reduces the risk of breast cancer.

DES—Women who took diethylstilbestrol (DES) to prevent miscarriage may have an increased risk of breast cancer after age 40.

Hormone replacement therapy (HRT)—A higher risk for breast cancer exists for women who have received hormone replacement therapy for several years or more.

Obesity—Obesity has been linked to breast cancer, although this link is controversial.

Radiation—Radiation therapy received as a child or young adult to treat cancer of the chest area increases the risk of developing breast cancer.

Symptoms: Early breast cancer usually does not cause symptoms. As the cancer grows, symptoms may include: breast lump or lump in the armpit that is hard, has uneven edges, and usually does not hurt; change in the size, shape, or feel of the breast or nipple—for example, redness, dimpling, or puckering; fluid coming from the nipple—may be bloody, clear-to-yellow, or green, and look like pus. In men, symptoms of breast cancer include breast lump, breast pain and tenderness.

Symptoms of advanced breast cancer may include: bone pain, breast pain or discomfort, skin ulcers, swelling of one arm (next to breast with cancer), and weight loss.

Exams and Tests: A doctor will ask about symptoms and risk factors, and perform a physical exam, which includes both breasts, armpits, and the neck and chest area. Additional tests may include: mammography, breast MRI, breast ultrasound, breast biopsy, needle aspiration, or breast lump removal to remove all or part of the breast lump for closer examination. If a patient has breast cancer, additional tests are done to see if the cancer has spread, e.g., staging, to help guide future treatment.

Breast cancer stages range from 0 to IV. In general, breast cancer may be in situ (noninvasive) breast cancer or invasive breast cancer. The higher the number, the more advanced the cancer.

Treatment: Treatment is based on many factors, including type and stage of the cancer, whether the cancer is sensitive to certain hormones, and whether or not the cancer overproduces (overexpresses) a gene called HER2/neu. In general, cancer treatments may include: chemotherapy, radiation therapy, surgery to remove cancerous tissue—a lumpectomy removes the breast lump; mastectomy removes all or part of the breast and possible nearby structures. Further, an anti-Fn14 antibody described herein can be used to treat breast cancer, alone or in combination with another treatment described herein. Other treatments include: hormonal therapy and targeted therapy. An example of hormonal therapy is the drug tamoxifen. This drug blocks the effects of estrogen, which can help breast cancer cells survive and grow. Most women with estrogen sensitive breast cancer benefit from this drug. A newer class of medicines called aromatase inhibitors, such as exemestane (Aromasin), have been shown to work just as well or even better than tamoxifen in post-menopausal women with breast cancer. Targeted therapy uses special anti-cancer drugs that identify certain changes in a cell that can lead to cancer. One such drug is trastuzumab (HERCEPTIN®). For women with stage IV HER2-positive breast cancer, HERCEPTIN® plus chemotherapy has been shown to be work better than chemotherapy alone. Studies have also shown that in women with early stage HER2-positive breast cancer, this medicine plus chemotherapy cuts the risk of the cancer coming back by 50%. An anti-Fn14 antibody described herein can be used to treat in combination with HERCEPTIN® (alone or with chemotherapy).

Cancer treatment may be local or systemic. Radiation and surgery are forms of local treatment. Chemotherapy is a type of systemic treatment.

Most women receive a combination of treatments. For women with stage I, II, or III breast cancer, the main goal is to treat the cancer and prevent it from returning. For women with stage IV cancer, the goal is to improve symptoms and help them live longer. In most cases, stage IV breast cancer cannot be cured. An anti-Fn14 antibody described herein can be used, alone or in combination with another treatment described herein, to treat stage 0, I, II, III, or IV breast cancer.

Stage 0-Lumpectomy plus radiation or mastectomy is the standard treatment.

Stage I and II—Lumpectomy plus radiation or mastectomy with some sort of lymph node removal is standard treatment. Hormone therapy, chemotherapy, and biologic therapy may also be recommended following surgery.

Stage III—Treatment involves surgery possibly followed by chemotherapy, hormone therapy, and biologic therapy.

Stage IV—Treatment may involve surgery, radiation, chemotherapy, hormonal therapy, or a combination of such treatments.

The 5-year survival rates for persons with breast cancer that is appropriately treated are as follows:

100% for stage 0

100% for stage I

92% for stage IIA

81% for stage IIB

67% for stage IIIA

54% for stage IIIB

20% for stage IV

Possible Complications Breast cancer can spread to other parts of the body. Sometimes, cancer returns even after the entire tumor is removed and nearby lymph nodes are found to be cancer-free. Side effects or complications from cancer treatment are possible. For example, radiation therapy may cause temporary swelling of the breast, and aches and pains around the area.

Prevention: A healthy diet and a few lifestyle changes may reduce your overall chance of cancer in general.

Breast cancer is more easily treated and often curable if it is found early. Early detection involves: breast self-exams (BSE), clinical breast exams by a medical professional, and/or screening mammography.

### Pharmaceutical Compositions

An anti-Fn14 antibody (such as an antibody described herein) can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

In one embodiment, the anti-Fn14 antibody is formulated with excipient materials, such as sodium chloride, sodium dibasic phosphate heptahydrate, sodium monobasic phosphate, and a stabilizer. It can be provided, for example, in a buffered solution at a suitable concentration and can be stored at 2-8° C.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the anti-Fn14 antibody may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

An anti-Fn14 antibody can be modified, e.g., with a moiety that improves its stabilization and/or retention in circulation, e.g., in blood, serum, or other tissues, e.g., by at least 1.5, 2, 5, 10, or 50 fold.

For example, the anti-Fn14 antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used.

For example, the anti-Fn14 antibody can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

In some implementations, the anti-Fn14 antibody can also be coupled to or otherwise associated with a label or other agent, e.g., another therapeutic agent such as a cytotoxic or cytostatic agent, although, in many embodiments, this configuration is unnecessary. Examples of cytotoxic and chemotherapeutic agents include taxol, cytochalasin B, gramicidin D, vinblastine, doxorubicin, daunorubicin, a maytansinoid (e.g., maytansinol or the DM1 maytansinoid, a sulfhydryl-containing derivative of maytansine), mitoxantrone, mithramycin, actinomycin D, 1-dehydrotestosterone, glucocorticoids, procaine, taxane, tetracaine, lidocaine, propranolol, and puromycin and analogs or homologs thereof.

When the anti-Fn14 antibody is used in combination with a second agent (e.g., a chemotherapeutic agent), the two agents can be formulated separately or together. The agents can be formulated or otherwise used in a synergistically effective amount. It is also possible to use one or both of the agents in amounts less than would be used for mono-therapy. For example, the respective pharmaceutical compositions can be mixed, e.g., just prior to administration, and administered together or can be administered separately, e.g., at the same or different times.

It is also possible to use other Fn14-binding or agonist agents. The agent may be any type of compound (e.g., small organic or inorganic molecule, nucleic acid, protein, or peptide mimetic) that can be administered to a subject. In one embodiment, the agent is a biologic, e.g., a protein having a molecular weight of between 5-300 kDa. For example, an Fn14 agonist agent may activate events downstream of Fn14 engagement. Exemplary Fn14 agonist agents, other than agonist antibodies that bind to Fn14, include TWEAK and soluble forms of TWEAK (see e.g., U.S. Pat. No. 7,109,298). Such agents can be administered as part of a combination therapy with one or more antibodies described herein. Other therapeutic agents described herein can also be provided as a pharmaceutical composition, e.g., by standard methods or method described herein.

### Administration

The anti-Fn14 antibody can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration may be directly to a site of a cancer, e.g., into and/or adjacent to a tumor. In some cases, administration can be oral.

The route and/or mode of administration of the antibody can also be tailored for the individual case, e.g., by monitoring the subject, e.g., using tomographic imaging, e.g., to visualize a tumor.

The antibody can be administered as a fixed dose, or in a mg/kg dose. The dose can also be chosen to reduce or avoid production of antibodies against the anti-Fn14 antibody. Dosage regimens are adjusted to provide the desired response, e.g., a therapeutic response or a combinatorial therapeutic effect. Generally, doses of the anti-Fn14 antibody (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used.

A composition may comprise about 10 to 100 mg/ml or about 50 to 100 mg/ml or about 100 to 150 mg/ml or about 100 to 200 mg/ml of antibody.

In certain embodiments, the anti-Fn14 antibody in a composition is predominantly in monomeric form, e.g., at least about 90%, 92%, 94%, 96%, 98%, 98.5% or 99% in monomeric form. Certain anti-Fn14 antibody compositions may comprise less than about 5, 4, 3, 2, 1, 0.5, 0.3 or 0.1% aggregates, as detected, e.g., by UV at A280 nm. Certain anti-Fn14 antibody compositions comprise less than about 5, 4, 3, 2, 1, 0.5, 0.3, 0.2 or 0.1% fragments, as detected, e.g., by UV at A280 nm.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion.

An anti-Fn14 antibody dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments. Animal models can also be used to determine a useful dose, e.g., an initial dose or a regimen.

If a subject is at risk for developing cancer or other disorder described herein, the antibody can be administered before the full onset of the cancer or disorder, e.g., as a preventative measure. The duration of such preventative treatment can be a single dosage of the antibody or the treatment may continue (e.g., multiple dosages). For example, a subject at risk for the disorder or who has a predisposition for the disorder may be treated with the antibody for days, weeks, months, or even years so as to prevent the disorder from occurring or fulminating.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### Devices and Kits for Therapy

Pharmaceutical compositions that include the anti-Fn14 antibody can be administered with a medical device. The device can designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include anti-Fn14 antibody, and can be configured to deliver one or more unit doses of the antibody. The device can be further configured to administer a second agent, e.g., a chemo therapeutic agent, either as a single pharmaceutical composition that also includes the anti-Fn14 antibody or as two separate pharmaceutical compositions.

The pharmaceutical composition may be administered with a syringe. The pharmaceutical composition can also be administered with a needleless hypodermic injection device, such as the devices disclosed in U.S. Pat. No. 5,399,163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

An anti-Fn14 antibody can be provided in a kit. In one embodiment, the kit includes (a) a container that contains a composition that includes anti-Fn14 antibody, and optionally (b) informational material. The informational material can be descriptive, instructional, marketing or other material that relates to the methods described herein and/or the use of the agents for therapeutic benefit.

In an embodiment, the kit also includes a second agent for treating a disorder described herein, e.g., a chemotherapeutic agent. For example, the kit includes a first container that contains a composition that includes the anti-Fn14 antibody, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the anti-Fn14 antibody, e.g., in a suitable dose, dosage form, or mode of administration (e.g., a dose, dosage form, or mode of administration described herein), to treat a subject who has had or who is at risk for a cancer, or other disorder described herein. The information can be provided in a variety of formats, include printed text, computer readable material, video recording, or audio recording, or information that provides a link or address to substantive material, e.g., on the internet.

In addition to the antibody, the composition in the kit can include other ingredients, such as a solvent or buffer, a stabilizer, or a preservative. The antibody can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the anti-Fn14 antibody and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

### Targeting Fn14-Expressing Cells

The anti-Fn14 antibodies described herein can be used to target a payload to a Fn14-expressing cell or to a tissue or other structure associated with Fn14. For example, the antibodies can be attached to a virus or virus like particle that can deliver an exogenous gene (e.g., for gene therapy) or to a liposome, e.g., a liposome that encapsulates a therapeutic agent or exogenous gene. An exemplary method for using an antibody to target a virus is described in Roux et al. (1989) Proc Natl Acad Sci USA (1989) 86:9079-9083. See also, e.g., Curr Gene Ther. (2005) 5:63-70 and Hum Gene Ther. (2004) 15:1034-1044.

The anti-Fn14 antibodies of this invention may also be attached to liposomes containing a therapeutic agent such as a chemotherapeutic agent. Attachment of antibodies to liposomes may be accomplished by any known cross-linking agent such as heterobifunctional cross-linking agents that have been widely used to couple toxins or chemotherapeutic agents to antibodies for targeted delivery. For example, conjugation to liposomes can be accomplished using the carbohydrate-directed cross-linking reagent 4-(4-maleimidophenyl) butyric acid hydrazide (MPBH) (Duzgunes et al. (1992) J. Cell. Biochem. Abst. Suppl. 16E 77). Liposomes containing antibodies can also be prepared by well-known methods (See, e.g. DE 3,218,121; Epstein et al. (1985) Proc. Natl. Acad. Sci. USA, 82:3688-92; Hwang et al. (1980) Proc. Natl. Acad. Sci. USA, 77:4030-34; U.S. Pat. Nos. 4,485,045 and 4,544,545).

### Diagnostic Uses

Anti-Fn14 antibodies can be used in a diagnostic method for detecting the presence of Fn14, in vitro (e.g., a biological sample, such as tissue, biopsy) or in vivo (e.g., in vivo imaging in a subject). For example, human or effectively human anti-Fn14 antibodies can be administered to a subject to detect Fn14 within the subject. For example, the antibody can be labeled, e.g., with an MRI detectable label or a radiolabel. The subject can be evaluated using a means for detecting the detectable label. For example, the subject can be scanned to evaluate localization of the antibody within the subject. For example, the subject is imaged, e.g., by NMR or other tomographic means.

Examples of labels useful for diagnostic imaging include radiolabels such as ¹³¹I, ¹¹¹In, ¹²³I, ^{99m}Tc, ³²P, ³³P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes, can also be employed. The protein ligand can be labeled with such reagents using known techniques. For example, see Wensel and Meares (1983) Radioimmunoimaging and Radioimmunotherapy, Elsevier, N.Y. for techniques relating to the radiolabeling of antibodies and Colcher et al. (1986) Meth. Enzymol. 121: 802-816.

The subject can be “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65-85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

MRI Contrast Agents. Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EP0 502 814 A. Generally, the differences related to relaxation time constants T1 and T2 of water protons in different environments is used to generate an image. However, these differences can be insufficient to provide sharp high resolution images.

The differences in these relaxation time constants can be enhanced by contrast agents. Examples of such contrast agents include a number of magnetic agents, paramagnetic agents (which primarily alter T1) and ferromagnetic or superparamagnetic agents (which primarily alter T2 response). Chelates (e.g., EDTA, DTPA and NTA chelates) can be used to attach (and reduce toxicity) of some paramagnetic substances (e.g., Fe³⁺, Mn²⁺, Gd³⁺). Other agents can be in the form of particles, e.g., less than 10 μm to about 10 nm in diameter). Particles can have ferromagnetic, anti-ferromagnetic or superparamagnetic properties. Particles can include, e.g., magnetite (Fe₃O₄), γ-Fe₂O₃, ferrites, and other magnetic mineral compounds of transition elements. Magnetic particles may include one or more magnetic crystals with and without nonmagnetic material. The nonmagnetic material can include synthetic or natural polymers (such as sepharose, dextran, dextrin, starch and the like).

The anti-Fn14 antibodies can also be labeled with an indicating group containing the NMR-active ¹⁹F atom, or a plurality of such atoms inasmuch as (i) substantially all of naturally abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American, 246:78-88 to locate and image Fn14 distribution.

In another aspect, the disclosure provides a method for detecting the presence of Fn14 in a sample in vitro (e.g., a biological sample, such as serum, plasma, tissue, biopsy). The subject method can be used to diagnose a disorder, e.g., a cancer. The method includes: (i) contacting the sample or a control sample with the anti-Fn14 antibody; and (ii) evaluating the sample for the presence of Fn14, e.g., by detecting formation of a complex between the anti-Fn14 antibody and Fn14, or by detecting the presence of the antibody or Fn14. For example, the antibody can be immobilized, e.g., on a support, and retention of the antigen on the support is detected, and/or vice versa. A control sample can be included. A statistically significant change in the formation of the complex in the sample relative to the control sample can be indicative of the presence of Fn14 in the sample. Generally, an anti-Fn14 antibody can be used in applications that include fluorescence polarization, microscopy, ELISA, centrifugation, chromatography, and cell sorting (e.g., fluorescence activated cell sorting).

The following are examples of the practice of the invention. They are not to be construed as limiting the scope of the invention in any way.

### EXAMPLES

### Example 1

### Anti-Fn14 Antibodies

Anti-Fn14 antibodies P4A8, P3G5, P2D3, and P3D8 were raised in Fn14-deficient mice by administration of CHO cells expressing human surface Fn14 and boosted with Fn14-myc-His protein. This immunization strategy appeared necessary as earlier immunization strategies were unsuccessful. The antibodies bind to both human and cynomolgus Fn14 proteins in vitro. An alignment of the human (top) and cynomolgus (bottom) Fn14 proteins is as follows:

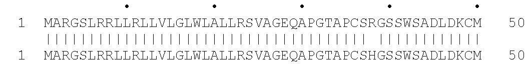

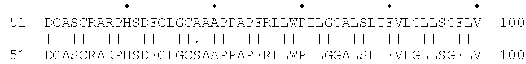

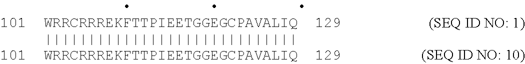

Properties of P4A8, which are further described below, include the following: monovalent binding affinity of about 1.6 or 2 nM; EC₅₀ for in vitro efficacy to trigger apoptosis of tumor cells is 170 μM; species cross-reactivity to human, cyno, rat and mouse Fn14; ability to induce tumor cell killing in vitro; efficacious in tumor xenograft models in vivo; induces NF-kB signaling and caspase-3/7 induction in vitro and in vivo; half-life in mice of 2 days; half-life in rats of >5 days; and does not bind to other TNF family member receptors.

### Example 2

### Anti-Fn14 Antibodies Kill Tumor Cells In Vitro

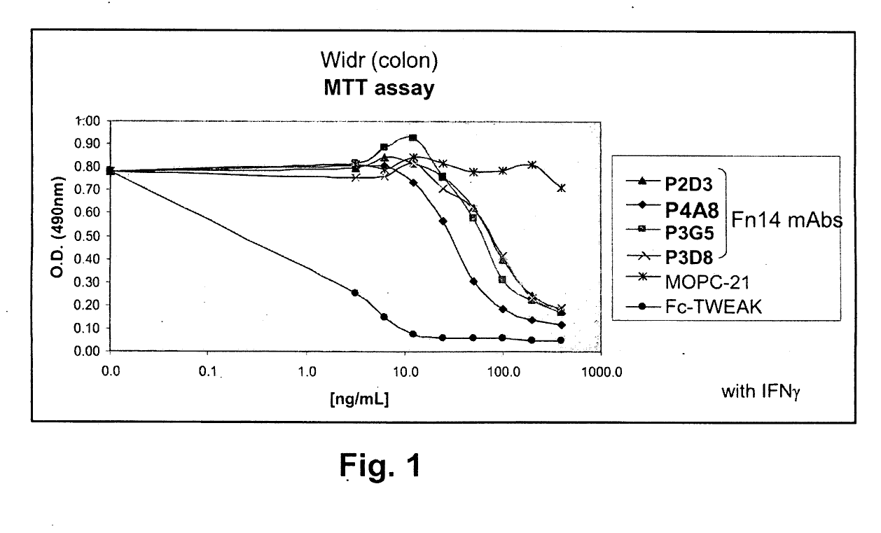

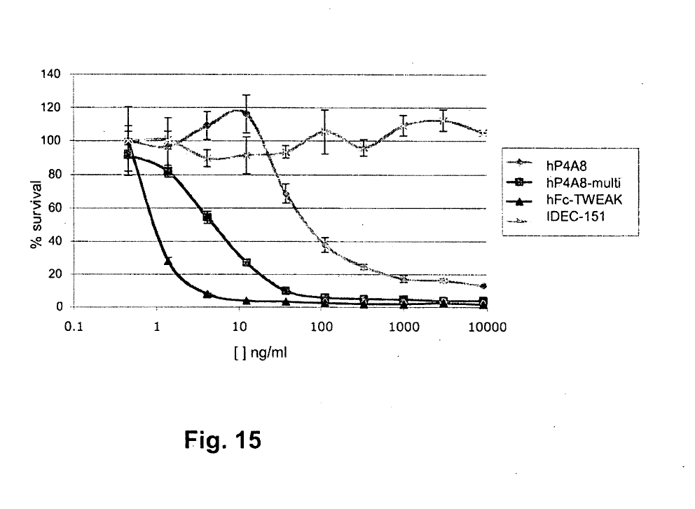

Widr colon cancer cells were treated with increasing concentrations of an anti-Fn14 antibody (P2D3, P4A8, P3G5, or P3D8), a positive control agonist (Fc-TWEAK), or a negative control (MOPC21), each in combination with IFN-γ. Cell death was measured by decreased viability as scored by an MTT assay. The antibodies P2D3, P4A8, P3G5, and P3D8 as well as Fc-TWEAK were able to kill the tumor cells (FIG. 1). The EC50 of P4A8 in the WiDr MTT assay is about 30 ng/ml. Similar results were obtained with humanized P4A8IgG1 (hP4A8IgG1; described below) in the MTT assay. In addition, treatment with a multimeric version of hP4A8IgG1 (generated by binding hP4A8IgG1 to Protein A) showed an enhanced effect (FIG. 15).

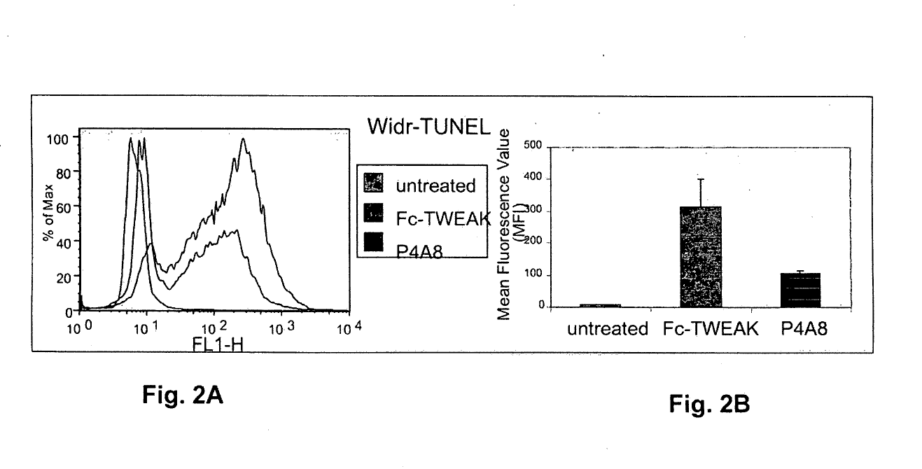

The ability of the P4A8 antibody to induce apoptosis of WiDr colon cancer cells in vitro was measured by TUNEL assay. WiDr cells were treated with the P4A8 antibody or a positive control (Fc-TWEAK), each in combination with IFN-γ, or were left untreated. Both the P4A8 antibody and Fc-TWEAK were able to kill the tumor cells (FIGS. 2A and 2B).

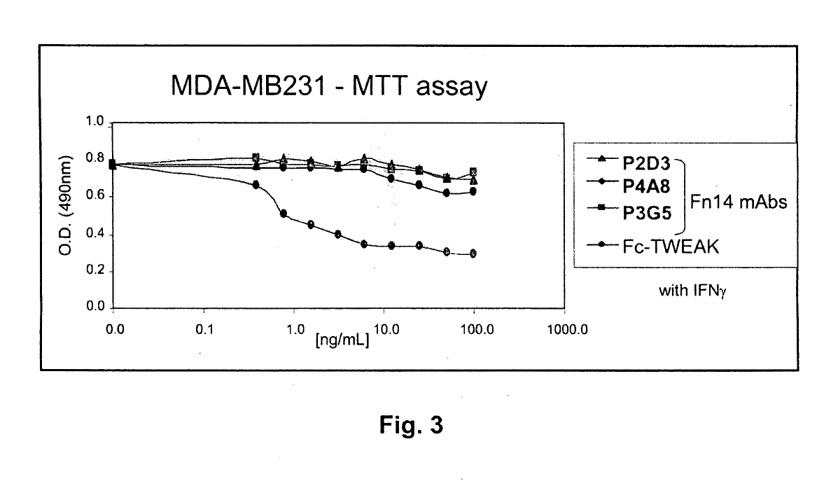

Anti-Fn14 antibodies were tested for their ability to kill MDA-MB231 breast cancer cells in vitro. The cancer cells were treated with increasing concentrations of the antibody P2D3, P4A8, P3G5, or P3D8, or a positive control agonist (Fc-TWEAK), each in combination with IFN-γ. Cell death was measured by decreased viability as scored by an MTT assay. The MDA-MB231 cells were resistant to the anti-Fn14 antibodies in vitro (FIG. 3).

P4A8 was rapidly internalized into all cells tested. The appearance of internal granules varied from small and numerous (WiDr) to large and few (MDA-MB231). In addition, P4A8 treatment of cells caused an induction or stabilization of Fn14 itself. This phenomenon was not due to an increase in Fn14 mRNA.

### Example 3

### Induction of Interleukin-8 Secretion

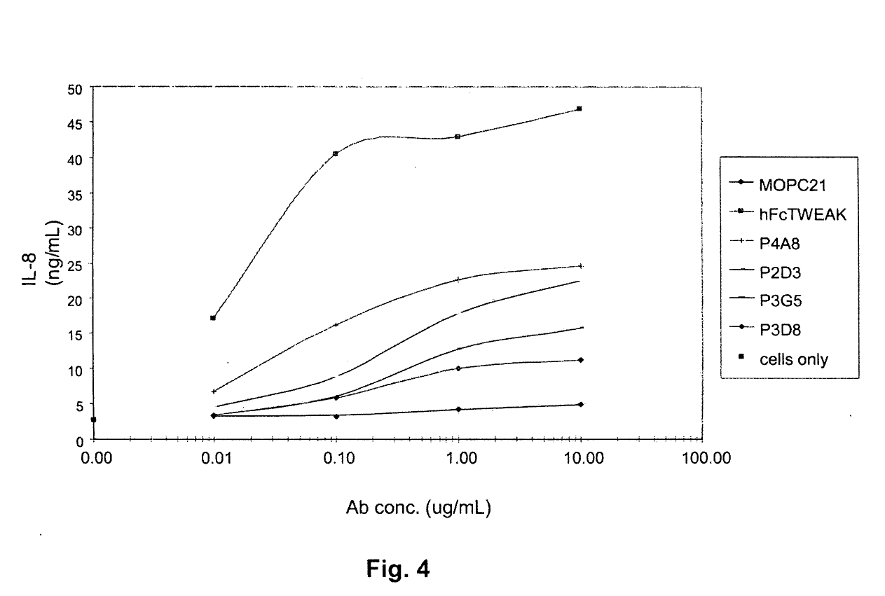

The P2D3, P4A8, P3G5, and P3D8 antibodies were tested to assess their ability to induce interleukin 8 (IL-8) secretion in vitro. A375 cells were treated with increasing concentrations of MOPC21 negative control, hFcTWEAK positive control, or P2D3, P4A8, P3G5, or P3D8 antibody. The levels of IL-8 secreted into the culture medium at each concentration was measured. Each of the antibodies induced IL-8 secretion and are thus capable of acting as Fn14 agonists (FIG. 4).

### Example 4

### Treatment of Tumors In Vivo

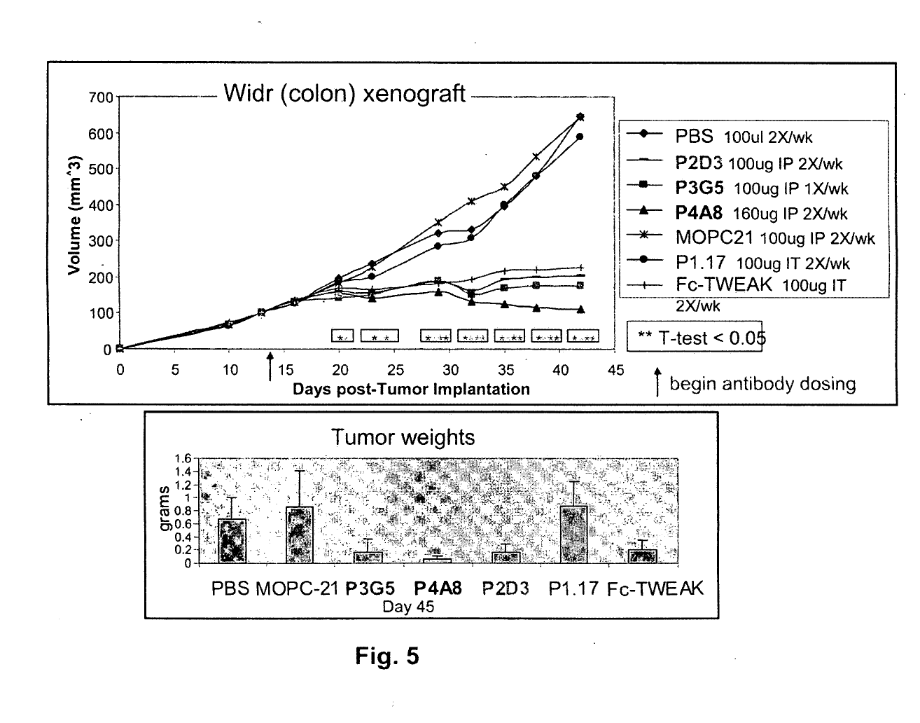

To test the ability of the anti-Fn14 antibodies to treat cancer in vivo, WiDr colon cancer cell xenografts were implanted into mice. After tumor implantation, the animals were treated with an anti-Fn14 antibody (P2D3, P4A8, P3G5, or P3D8), a negative control (PBS, MOPC21 or P1.17), or a positive control (Fc-TWEAK). The doses used, the routes of administration, and the frequency of administration are shown in FIG. 5. Tumor growth was measured by tumor volume (mm³, top panel) or tumor weight (grams, bottom panel). The anti-Fn14 antibodies were efficacious in treating tumors in vivo (FIG. 5).

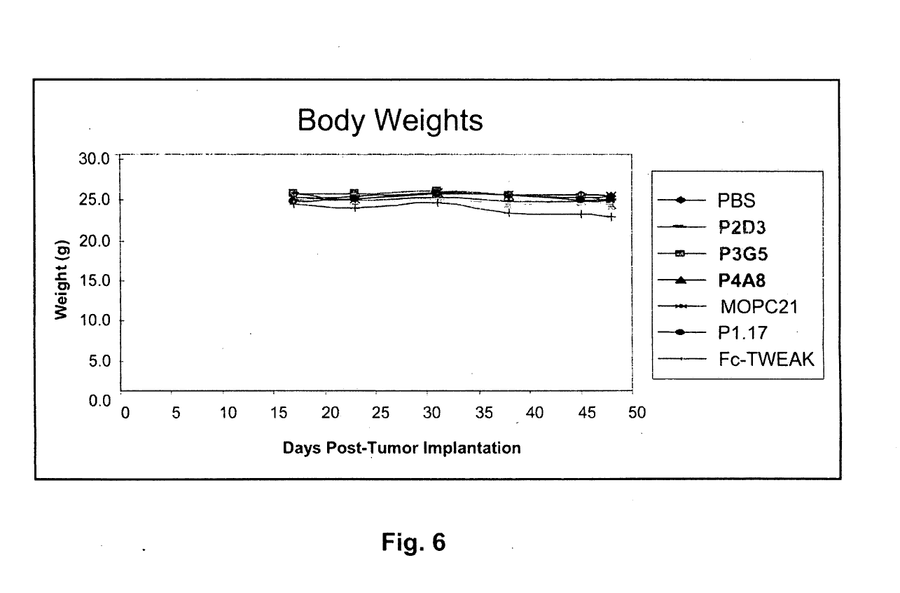

The anti-Fn14 antibodies and controls were also tested for toxicity. No obvious toxicities were observed with any of the treatments even after repeated doses, as measured by animal weight (FIG. 6).

### Example 5

### Treatment of Large Tumors

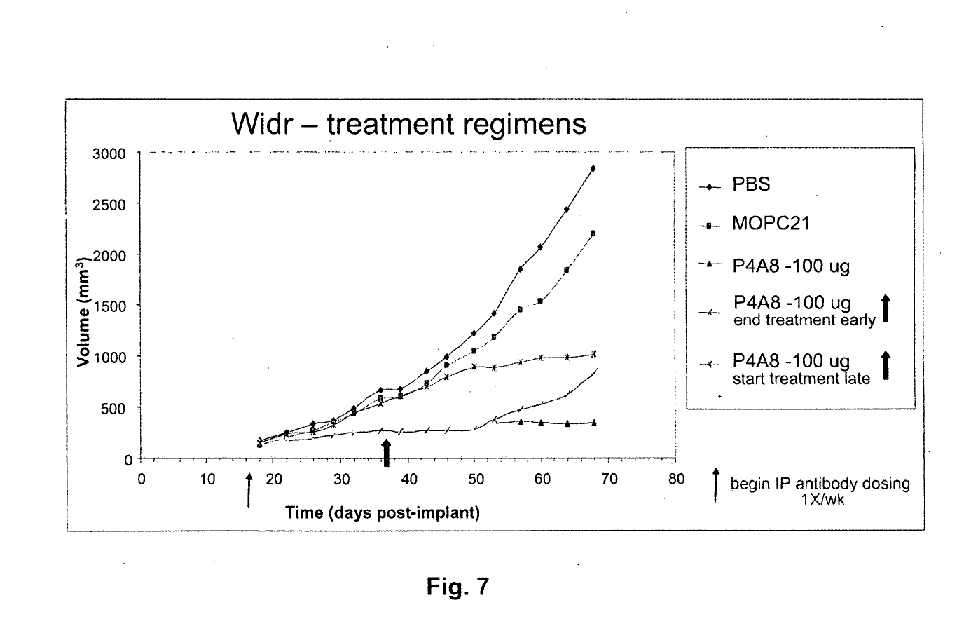

The ability of the anti-Fn14 antibodies to treat cancer in vivo was tested in large tumors. Widr colon cancer cell xenografts were implanted into mice. After tumor implantation, the animals were treated with an anti-Fn14 antibody (P4A8; 100 μg) or a negative control (PBS or MOPC21). Antibody was administered once a week and continued throughout the study, or dosing began on day 16 and ended early (day 37), or dosing began late (day 37) and ran through the end of the study. Tumor growth was measured by tumor volume (mm³). The anti-Fn14 antibodies were efficacious in treating tumors in vivo, even when treatment started late or was terminated early (FIG. 7).

### Example 6

### Dose Response

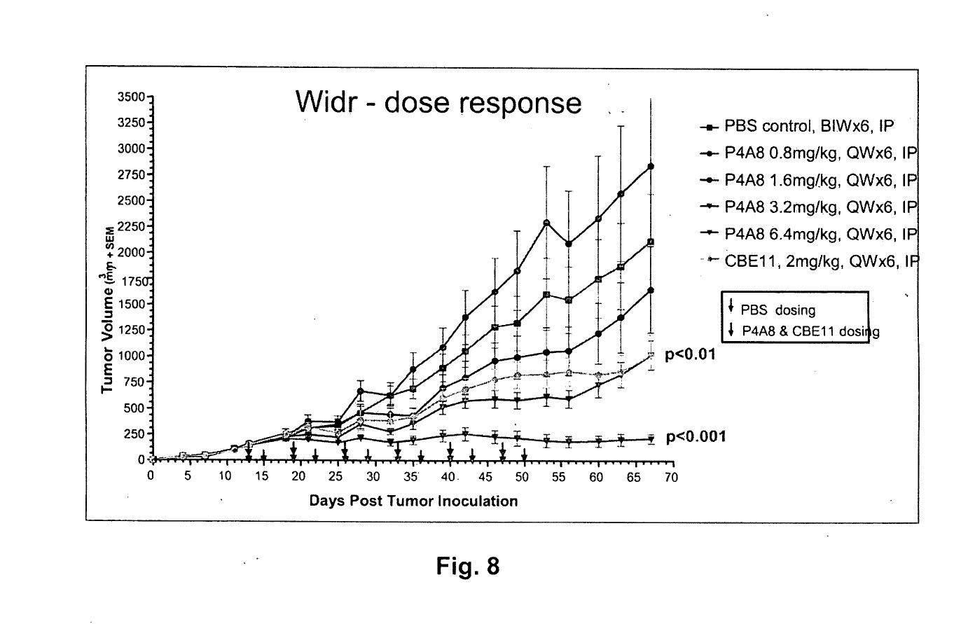

The dose response of a WiDr cell xenograft was examined. Various doses of P4A8 anti-Fn14 antibody and PBS negative control were tested (tumor volume (mm³) over time (days)). Efficacy increased with increasing doses of antibody (FIG. 8).

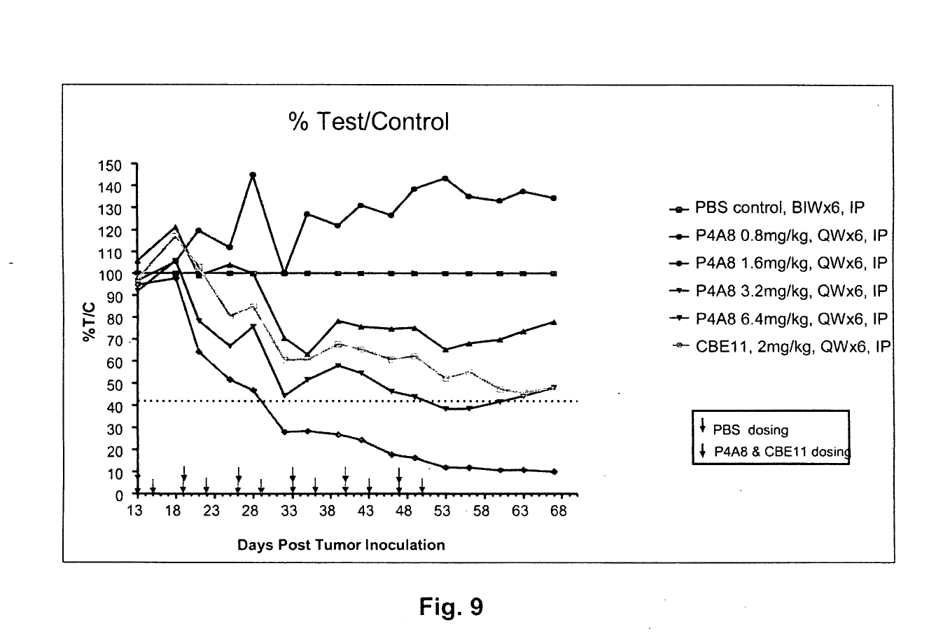

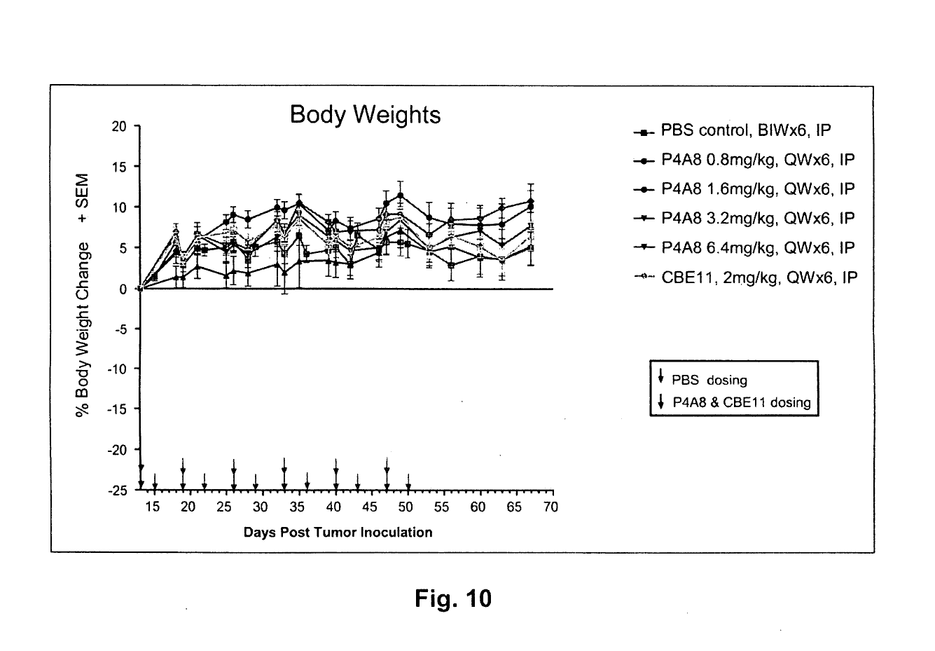

The dose response was also analyzed as a percent of test/control (% T/C). As shown in FIG. 9, efficacy increased with increasing doses of antibody. The various doses of the antibody and the controls were also tested for toxicity. No obvious toxicities were observed with any of the treatments even after repeated doses, as measured by percent body weight change (FIG. 10).

### Example 7

### Treatment of Breast Cancer Cell Tumors In Vivo

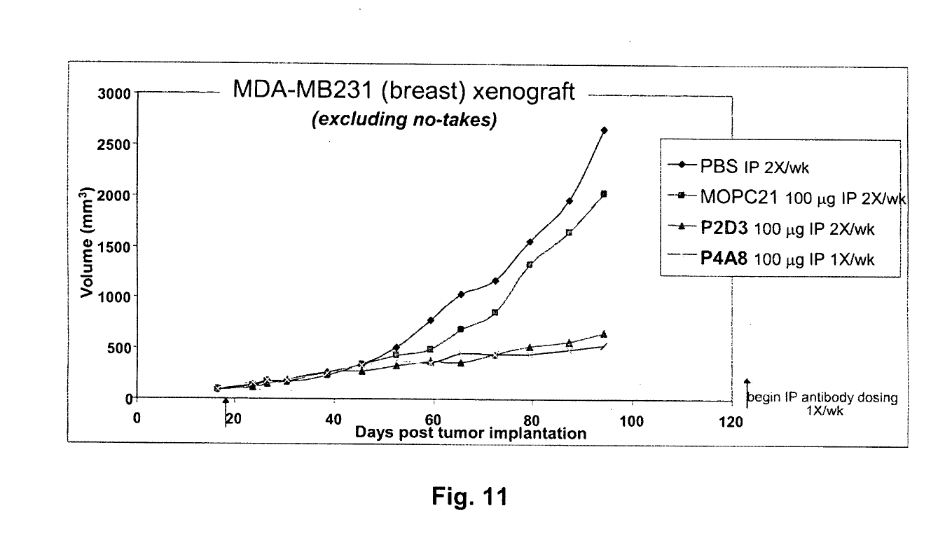

To test the ability of the anti-Fn14 antibodies to treat cancer in vivo, MDA-MB231 breast cancer cell xenografts were implanted into mice. After tumor implantation, the animals were treated with an anti-Fn14 antibody (P2D3 or P4A8) or a negative control (PBS or MOPC21). The doses used, the routes of administration, and the frequency of administration are shown in FIG. 11. Tumor growth was measured by tumor volume (mm³). The anti-Fn14 antibodies were efficacious in treating tumors in vivo (FIG. 11).

### Example 8

### Antibody Cross Reactivity

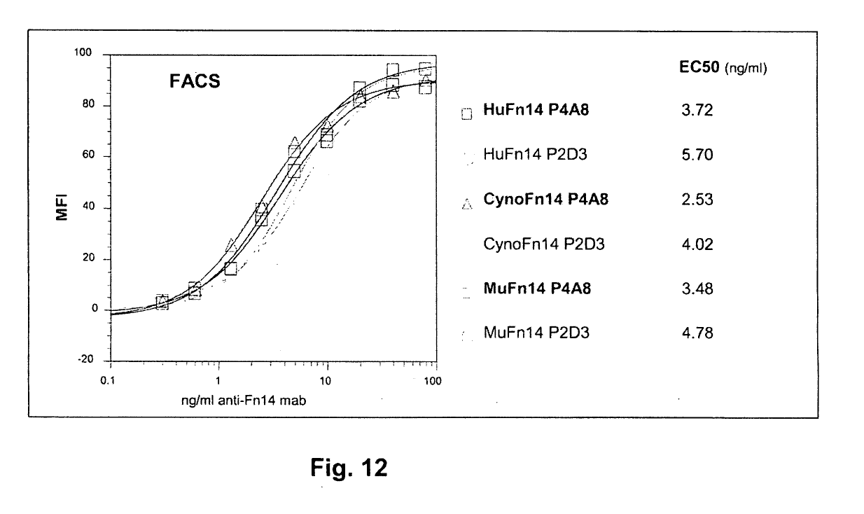

Anti-Fn14 antibodies P4A8 and P2D3 are cross reactive to Fn14 from multiple species. As shown in FIG. 12, both antibodies react with human, cynomolgus, and murine Fn14, as determined by flow cytometry (mean fluorescence value, MFI). EC50 values are also provided in the figure. P4A8 was also cross-reactive with rat Fn14. Rhesus monkey Fn14 was cloned and determined to be identical to human Fn14. Therefore, the binding characteristics of the antibodies to rhesus monkey Fn14 are the same as those to human Fn14.

Full-length Fn14 cDNAs encoding human (NM_{—}016639), cynomolgus (see Example 1), mouse (NM_{—}013749), rat (NM_{—}181086) and Xenopus (NM_{—}001090171) Fn14 were engineered to remove extraneous 5′ and 3′ UTRs and add an identical optimized Kozak sequence, then were subcloned into pNE001, a fully sequence-confirmed pUC-based EBV expression vector derived from the Invitrogen expression vector pCEP4, in which heterologous gene expression is controlled by a CMV-IE promoter and an SV40 polyadenylation signal, but lacking the EBNA gene and the hygromycin resistance gene. Fn14 expression vectors (human: pEAG2121, cynomolgus monkey: pEAG2120, mouse: pEAG2126, rat: pEAG2275 and Xenopus: pEAG2237) were co-transfected into 293E cells at a 1:1 molar ratio with an EBV expression vector carrying an EGFP reporter. Cells were used in FACS at 2 days post-transfection, staining with monoclonal antibodies of interest (with dilution titration) and gating on green EGFP-positive living cells. This type of assay depends upon the cell surface density of Fn14 and therefore reflects apparent EC50 values for a given transfection: this direct binding assay does not determine true Kd values.

Shown below is an alignment of the full-length Fn14 deduced protein sequences of human, cynomolgus monkey, rat and mouse:

| | 1 50 | | |
|---|---|---|---|
| human | MARGslRRLl rLLVLGlwLa LLRsVAGEQA PGTAPCSrGS SWSADLDKCM | | |
| cyno | MARGslRRLl rLLVLGlwLa LLRsVAGEQA PGTAPCShGS SWSADLDKCM | | |
| mouse | MAsawpRsLp qiLVLGfgLv LmRaaAGEQA PGTsPCSSGS SWSADLDKCM | | |
| rat | MApGwpRpLp qLLVLGfgLv LiRatAGEQA PGnAPCSSGS SWSADLDKCM | | |
| Consensus | MARG--RRL- -LLVLG--L- LLR-VAGEQA PGTAPCSSGS SWSADLDKCM | | |
| | 51 100 | | |
| human | DCASCrARPH SDFCLGCAAA PPApFRLLWP ILGGALSLTf VLgLlSGFLV | | |
| cyno | DCASCrARPH SDFCLGCsAA PPApFRLLWP ILGGALSLTf VLgLlSGFLV | | |
| mouse | DCASCpARPH SDFCLGCAAA PPAhFRLLWP ILGGALSLvl VLaLvSsFLV | | |
| rat | DCASCpARPH SDFCLGCAAA PPAhFRmLWP ILGGALSLal VLaLvSGFLV | | |
| Consensus | DCASC-ARPH SDFCLGCAAA PPA-FRLLWP ILGGALSLT- VL-L-SGFLV | | |
| | 101 130 | Identity to huFn14 | |
| human | WRRCRRREKF TTPIEETGGE GCPaVALIQ* | 100.0 | (SEQ ID NO: 1) |
| cyno | WRRCRRREKF TTPIEETGGE GCPaVALIQ* | 98.5 | (SEQ ID NO: 10) |
| mouse | WRRCRRREKF TTPIEETGGE GCPgVALIQ* | 81.5 | (SEQ ID NO: 28) |
| rat | WRRCRRREKF TTPIEETGGE GCPgVALIQ* | 83.1 | (SEQ ID NO: 29) |
| Consensus | WRRCRRREKF TTPIEETGGE GCPgVALIQ* | | (SEQ ID NO: 30) |

Positions identical to the consensus are in upper case, while positions differing from consensus are in lower case. The predicted signal sequence extends from residues 1-27 and the predicted transmembrane domain extends from residues 79-101. Overall percentage identity to human Fn14 is indicated above.

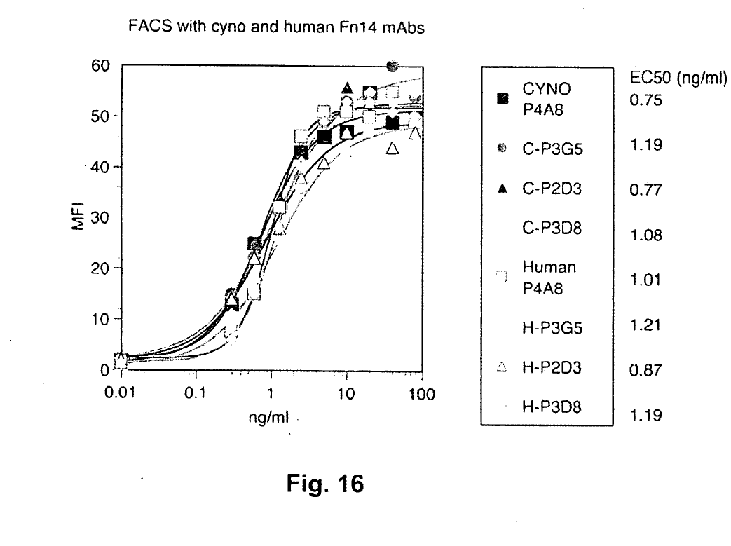

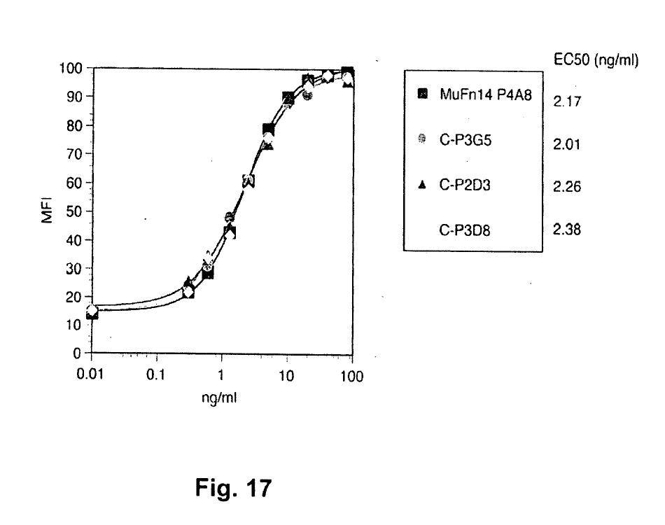

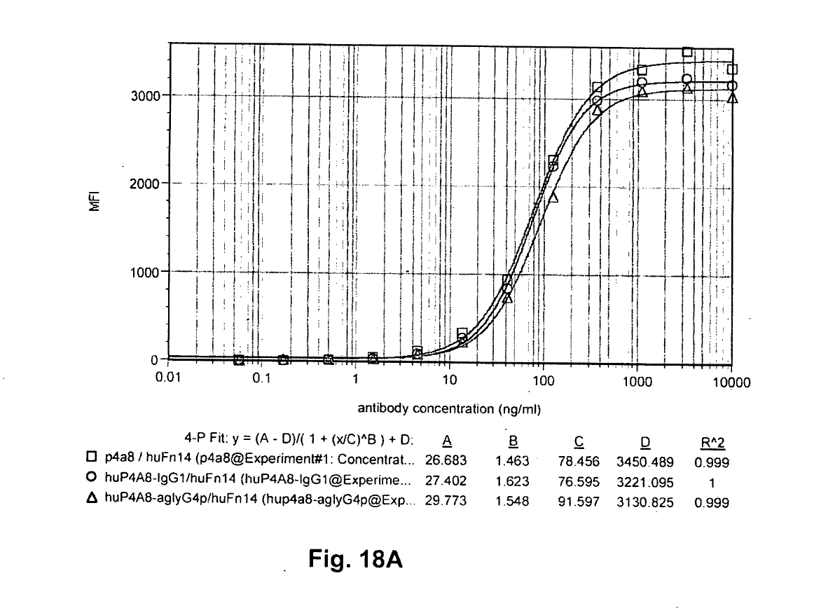

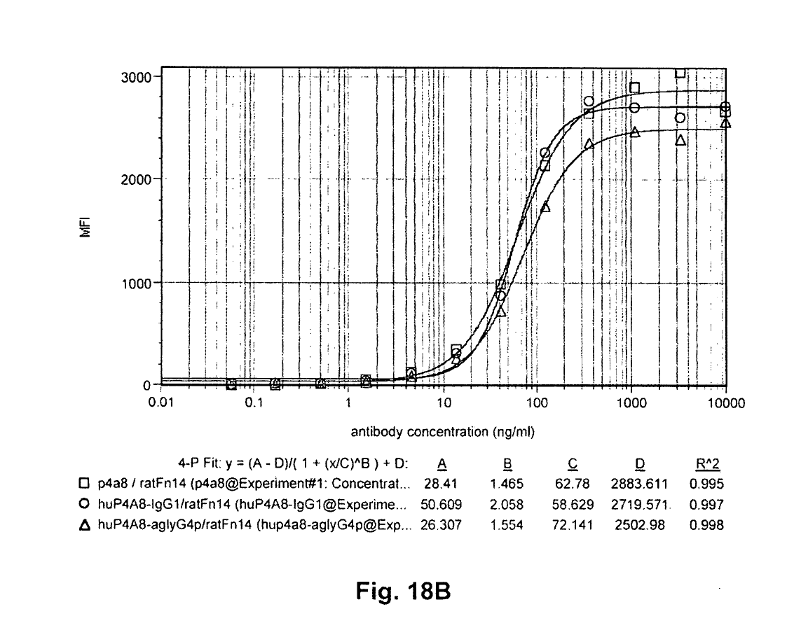

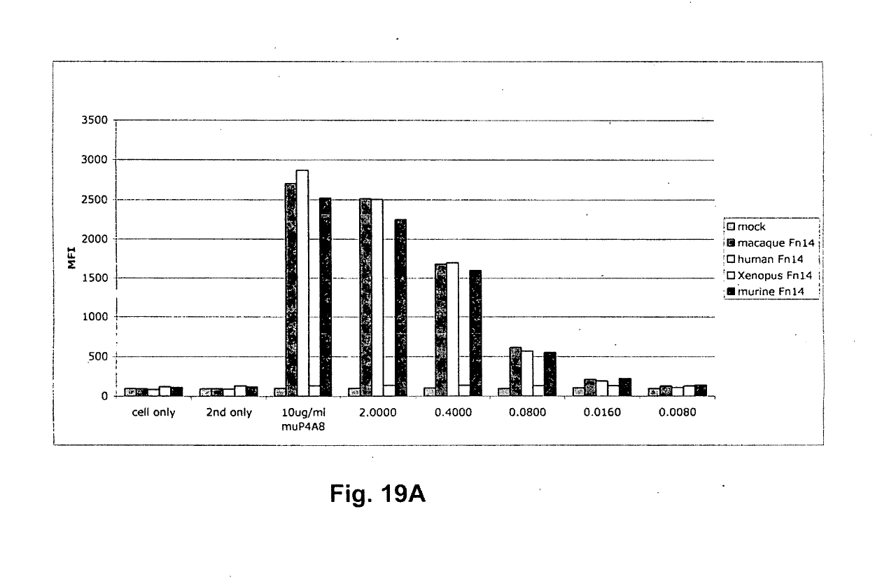

FIG. 16 shows direct binding FACS assay of the panel of anti-huFn14 mAbs P2D3, P3D8, P3G5 and P4A8 to human and cynomolgus monkey surface Fn14: all bind with similar EC50 values. FIG. 17 shows direct binding FACS assay of the panel of anti-huFn14 mAbs P2D3, P3D8, P3G5 and P4A8 to murine surface Fn14: all bind with similar apparent EC50 values that are similar to those for primate Fn14 binding. Humanized P4A8 (H1/L1) (huP4A8) (described below) binds to human Fn14 with an affinity equivalent to that of authentic murine P4A8 mAb. FIG. 18A and FIG. 18B show direct binding FACS data for variants of huP4A8 with different heavy chain effector function on human or rat Fn14, respectively: similar apparent EC50s are observed for huP4A8 binding to human and rat Fn14.

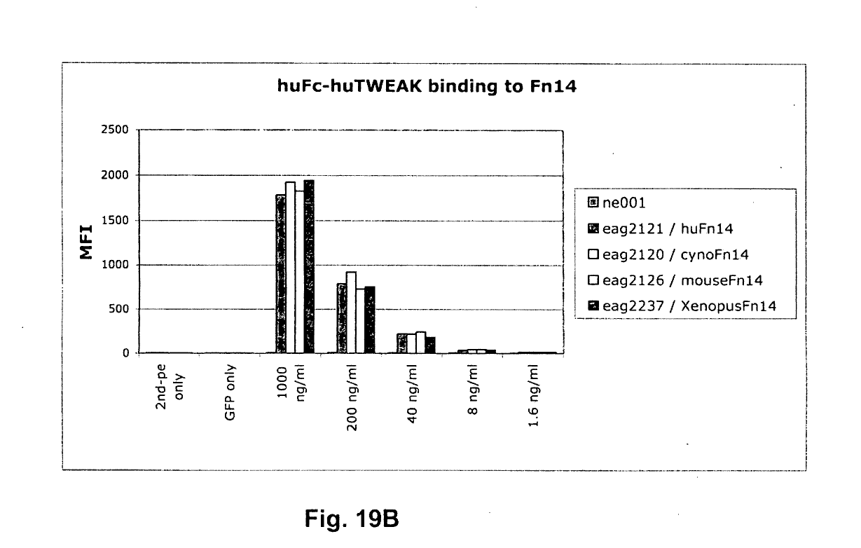

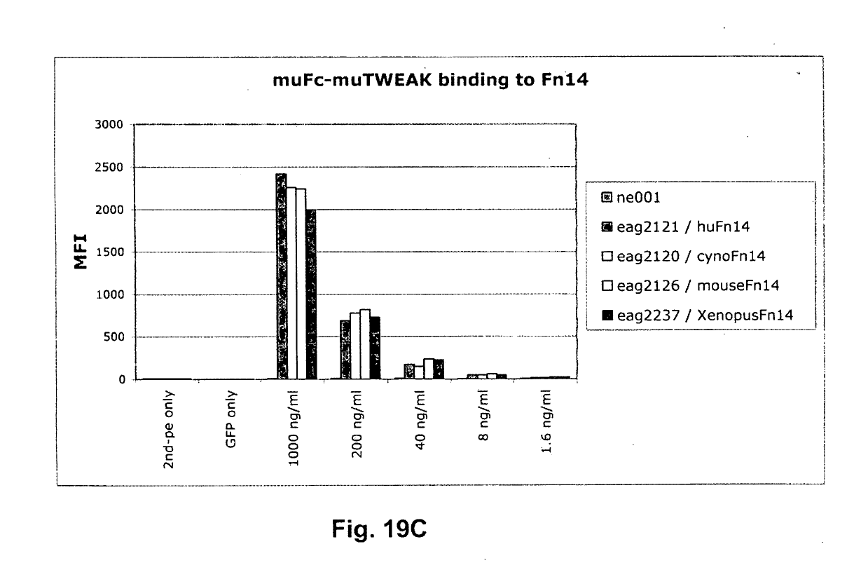

FIG. 19A shows that although P4A8 binds well to human, cynomolgus monkey and mouse surface Fn14, no binding to Xenopus Fn14 can be detected. FIG. 19B and FIG. 19C show that both Fc-huTWEAK and muFc-muTWEAK fusion proteins bind well to human, cynomolgus monkey, mouse and Xenopus surface Fn14, indicating that P4A8's failure to bind to Xenopus Fn14 is not due to a defect in surface presentation of its Fn14. Shown below is the gapped alignment between human (top) and Xenopus (bottom) Fn14, which share 48.3% similarity and only 40.8% identity:

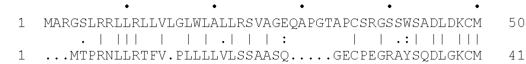

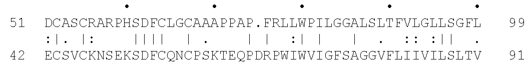

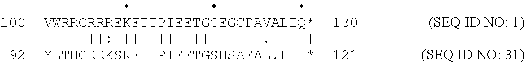

These results suggest that the P4A8 binding site is similar, but subtly different from the TWEAK binding site on Fn14.

It has also been shown that P4A8 does not bind to other TNF family receptors, and in this respect, it is selective for Fn14.

### Example 9

### Mapping the P4A8 Epitope to Fn14 Residue W42 (Sensitivity of P4A8 to W42A Mutation)

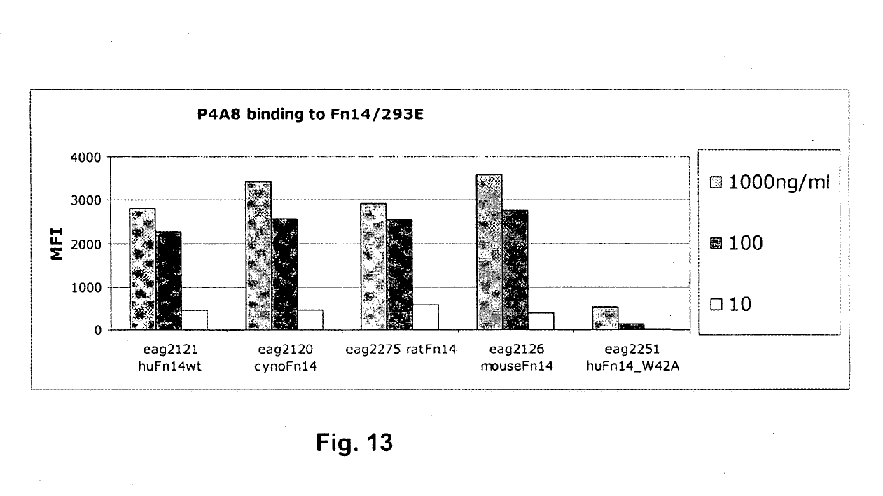

293E cells were transfected with nucleic acids encoding wildtype human, cynomolgus, rat, mouse and a human Fn14 with a W42A mutation, Binding of P4A8 to these cells was determined by FACS. The results are shown in FIG. 13. As indicated in the histogram, P4A8 binds significantly less well to the human Fn14 protein having a W42A mutation relative to the wildtype human Fn14 protein. Similarly, the P3G5 antibody also binds significantly less well to the human Fn14 protein having a W42A mutation (not shown).

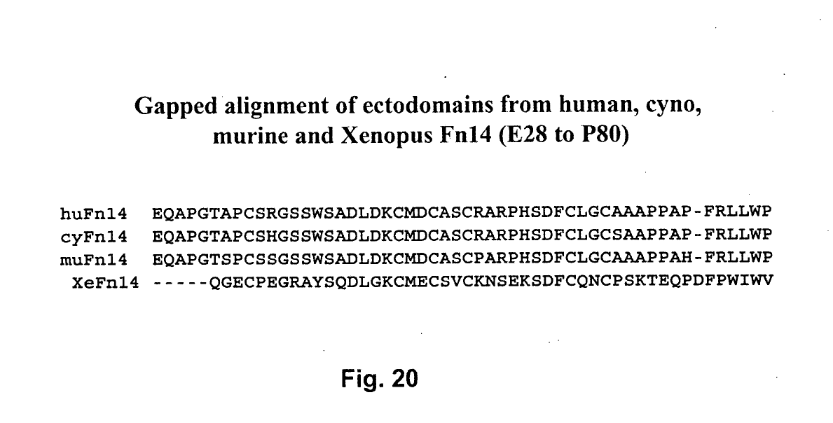

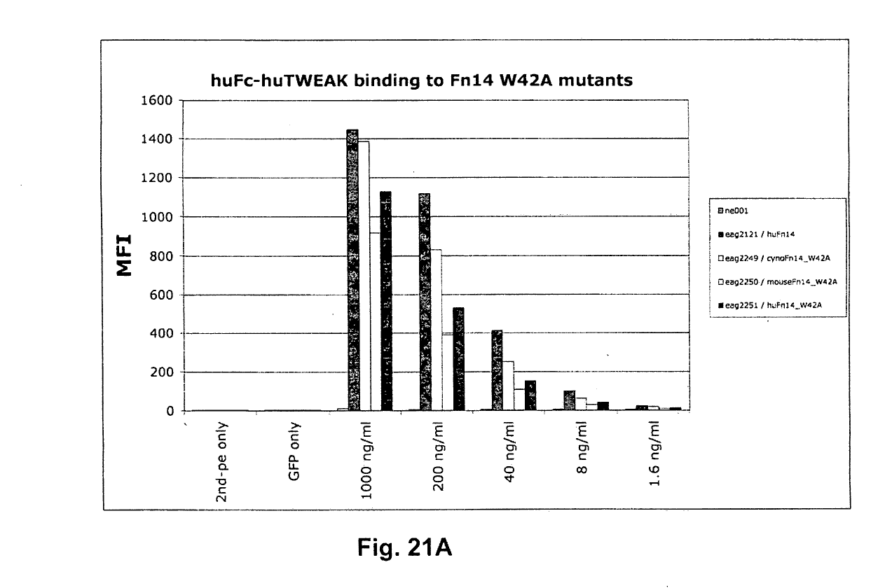

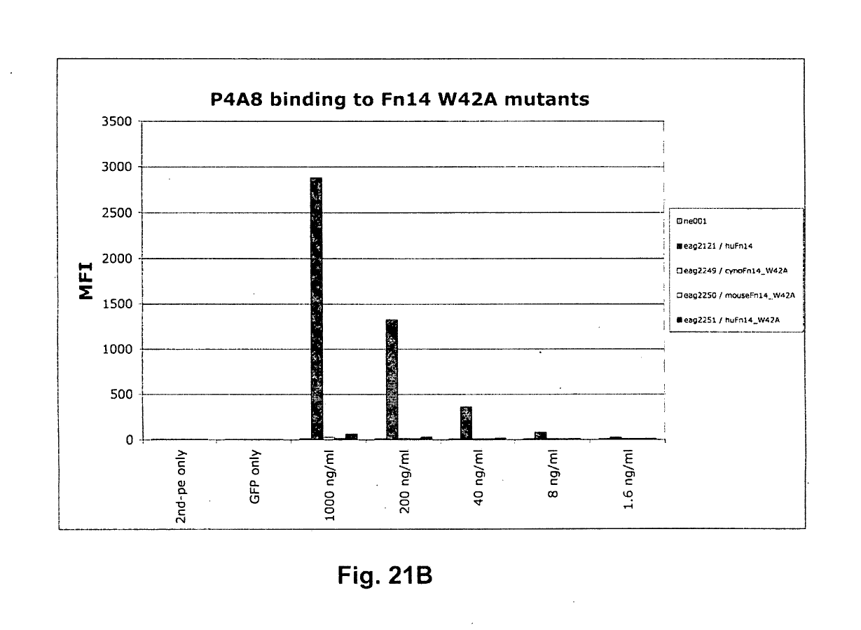

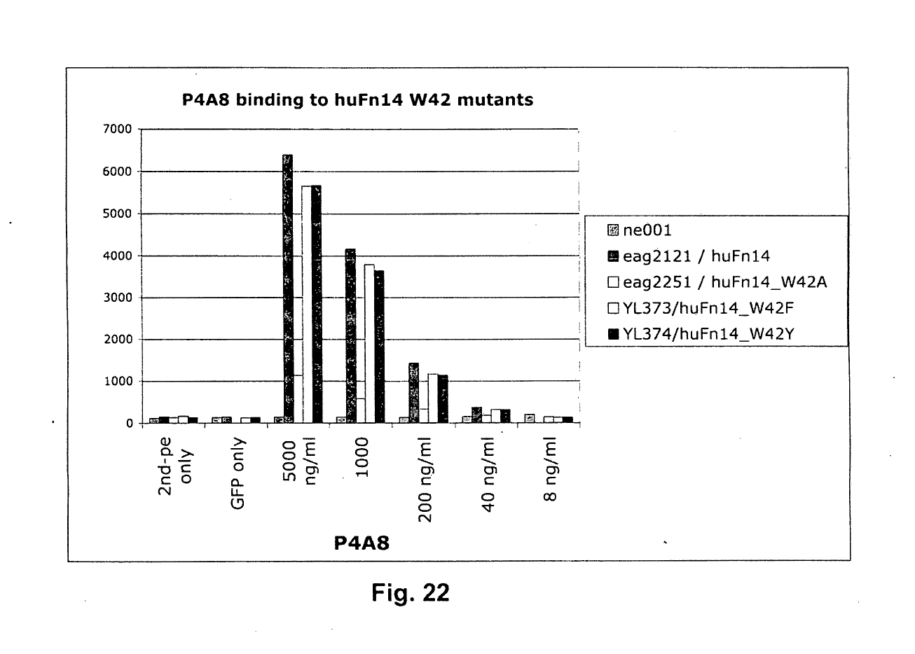

FIG. 20 is a gapped alignment of the Fn14 ectodomain (residues E28 to P80 to in human Fn14). W42A mutants were constructed in the EBV expression vectors for full-length human, cyno, and mouse Fn14 eDNAs by site-directed mutagenesis using Stratagene's QuikChange II kit following the manufacturer's recommended protocol. Mutated plasmids were identified by screening for introduced restriction site changes. The Fn14 cDNA sequences in the resultant plasmids were confirmed by DNA sequencing in the W42A mutant expression vectors: human Fn14 W42A designated pEAG2251, murine W42A designated pEAG2250, and cyno W42A designated pEAG2249. Wildtype huFn14 and W42A mutants in human, cyno, and murine Fn14 were over-expressed transiently in 293E cells and binding of Fc-TWEAK or P4A8 mAb assayed in FACS assay as previously described. FIG. 21A shows that Fc-TWEAK binds to all W42A mutants, while FIG. 21B shows that P4A8 binding is abrogated by mutation to W42A in all species examined. We performed site-directed mutagenesis on the huFn14 expression plasmid pEAG2121 to generate other point mutants for additional epitope mapping studies. FIG. 22 shows that P4A8 binding is restored to normal when residue W42 is mutated to large hydrophobic residues W42F or W42Y (pYL373 and pYL374, respectively).

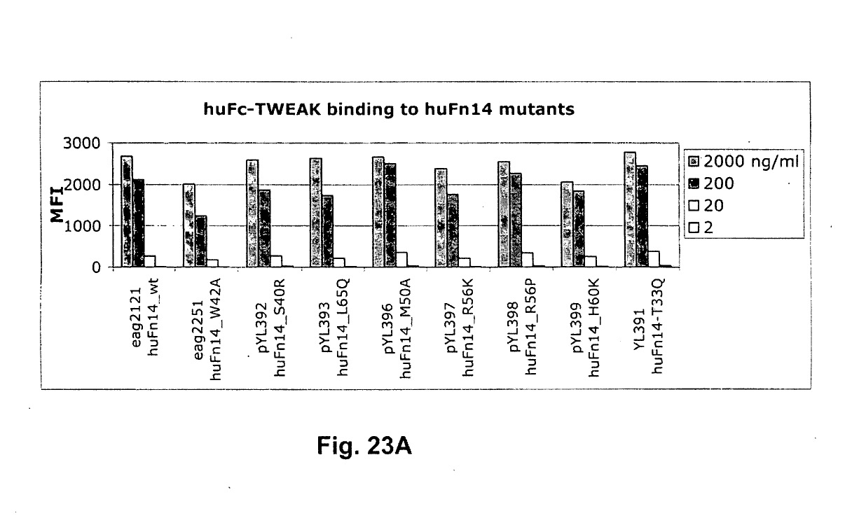

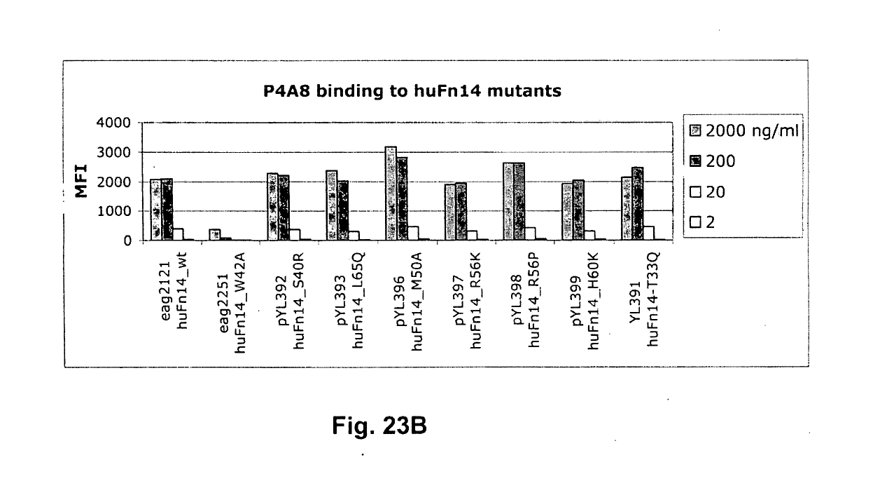

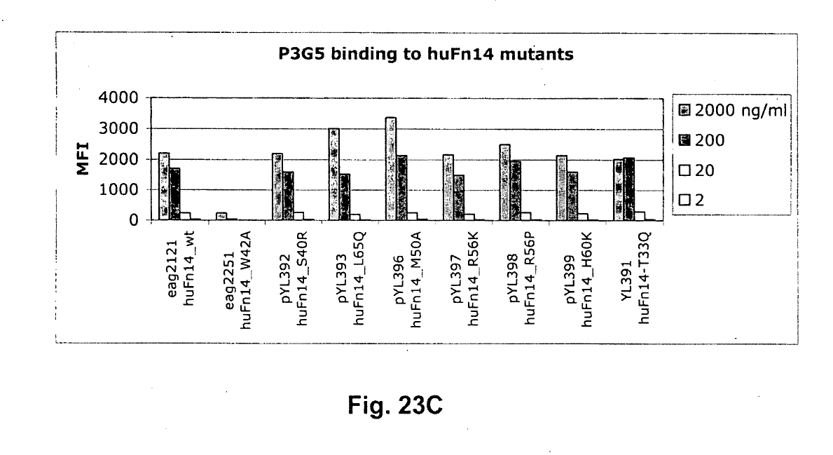

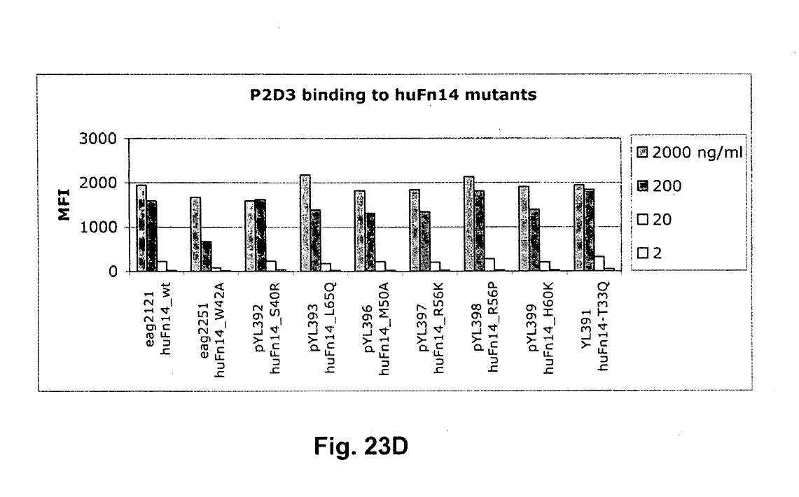

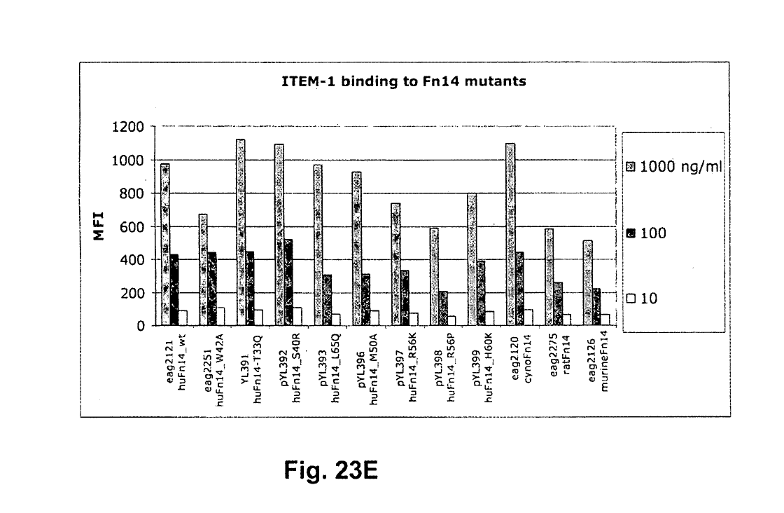

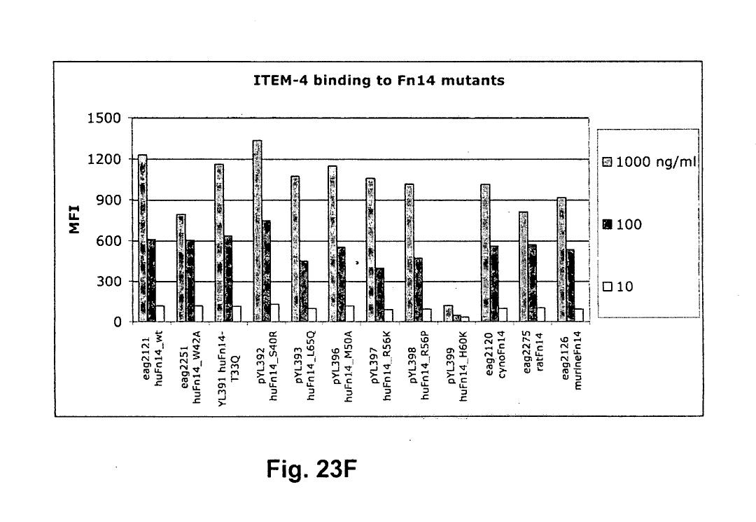

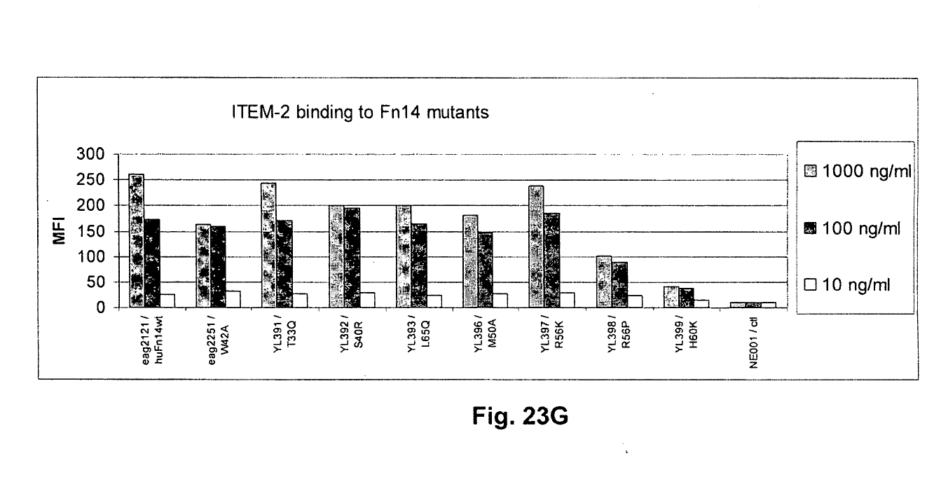

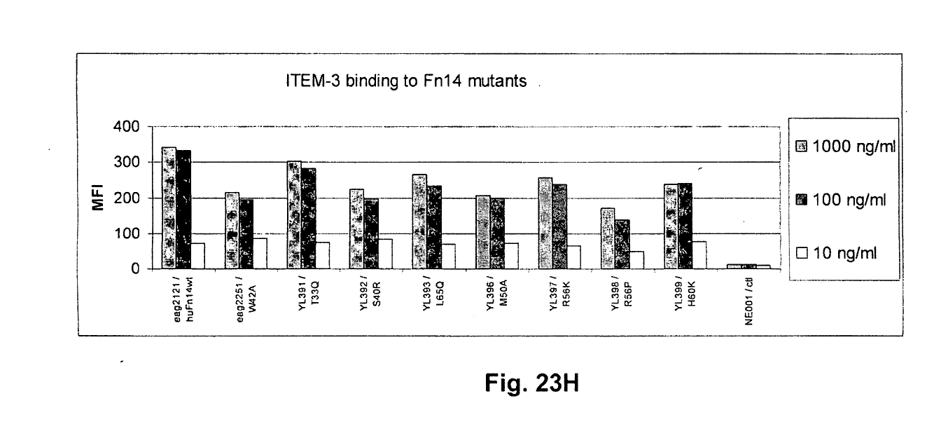

A panel of huFn14 point mutants was made by substituting Xenopus residues into the human sequence at a number positions by site-directed mutagenesis on the pEAG2121 template (EBV expression vector for huFn14): pYL391 T33Q, pYL392 S40R, pYL393 L65Q, pYL396 M50A, pYL397 R56K, pYL398 R56P (a more drastic substitution than the Xenopus change) and pYL399 H60K. Direct binding FACS assays showed that the entire mutant panel bound Fc-TWEAK (FIG. 23A). The agonist anti-Fn14 mAbs (P4A8, P3G5, P2D3 and P3D8) and ITEM-1, ITEM-2, ITEM-3, and ITEM-4 agonist mAbs described by Nakayama et al. (2003, J. Immunol. 170:341) were tested in direct binding FACS assay on human, cynomolgus monkey, rat, and mouse Fn14 and on the entire huFn14 mutant panel (W42A, T33Q, S40R, L65Q, M50A, R56K, R56P and H60K). P4A8 binding to the mutant panel is shown in FIG. 23B, P3G5 results are shown in FIG. 23C, P2D3 results are shown in FIG. 23D, ITEM-1 results are shown FIG. 23E, ITEM-4 results are shown in FIG. 23F, ITEM-2 results are shown in FIG. 23G, and ITEM-3 results are shown in FIG. 23H. The results indicate that P3G5 and P4A8 are sensitive to the Fn14 W42A substitution, but P2D3 (and P3D8) and the four ITEM anti-Fnl4 mAbs are insensitive to the W42A change. All of the antibodies tested bind to human, cynomolgus monkey, rat, and mouse Fn14.

### Example 10

### Immunohistochemistry

The anti-Fn14 antibody P4A8 was tested for use as an immunohistochemistry (IHC) reagent to detect Fn14 in sections of paraffin tissue sections. Paraffin sections were obtained for normal pancreatic tissue and pancreatic tumor tissue. P4A8 was able to stain Fn14 in the paraffin sections and the results demonstrated that Fn14 is overexpressed in pancreatic tumors as compared to normal tissue.

P4A8 was also used to measure Fn14 levels in normal tissue. Human tissue arrays (frozen and paraffin) were stained with P4A8. The results showed predominantly mild, but occasionally minimal or moderate staining of epithelial cells, endothelium and muscle, and a cytoplasmic distribution (membranes were not highlighted).

### Example 11

### Sequences of Anti-Fn14 Antibodies

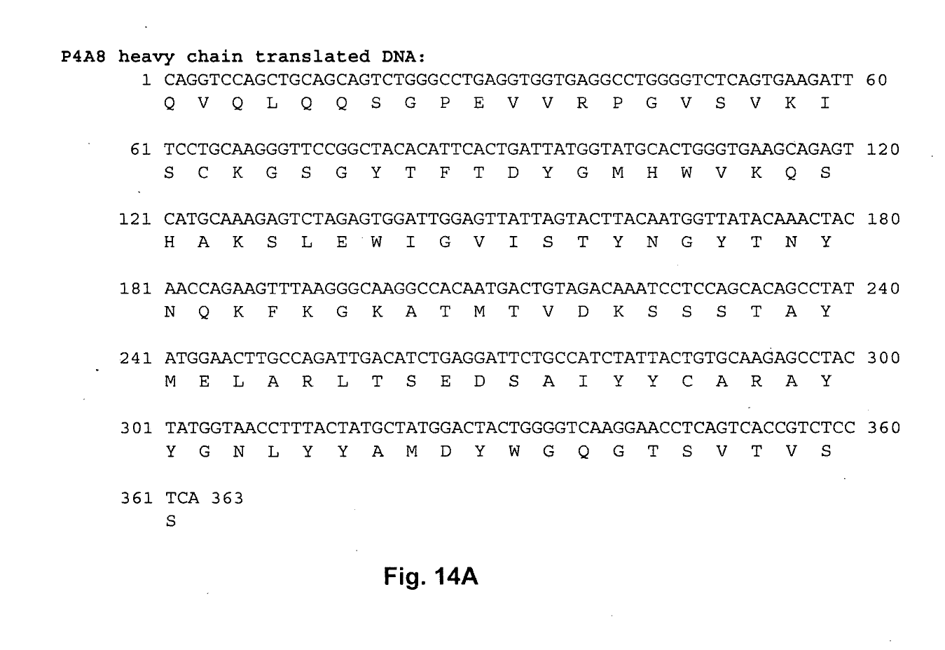

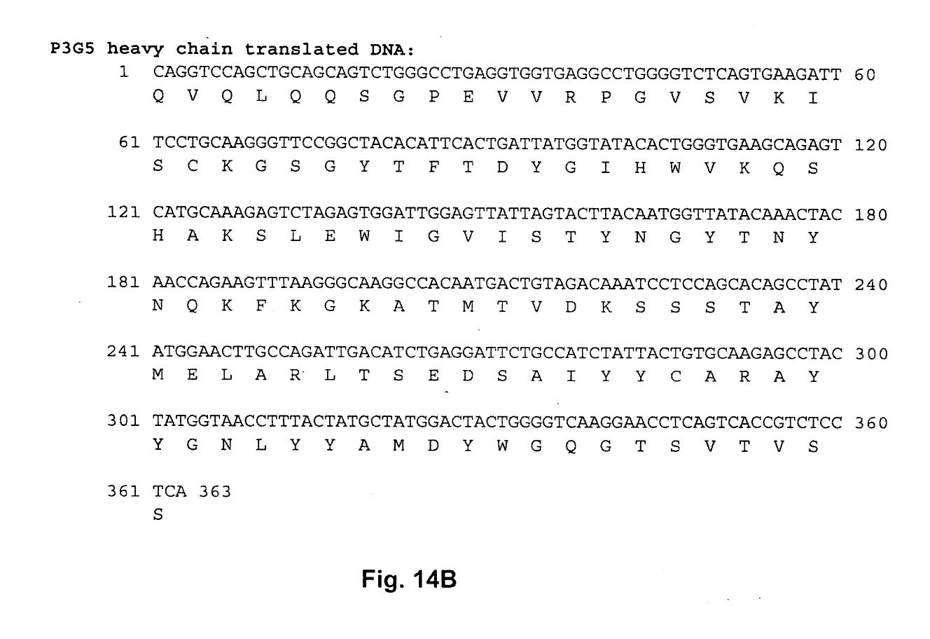

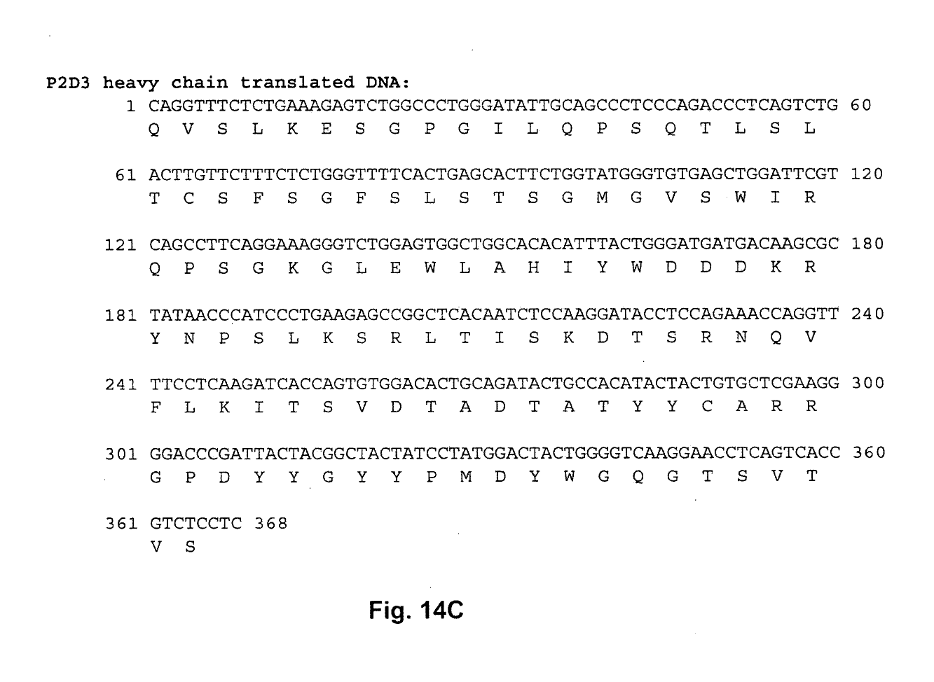

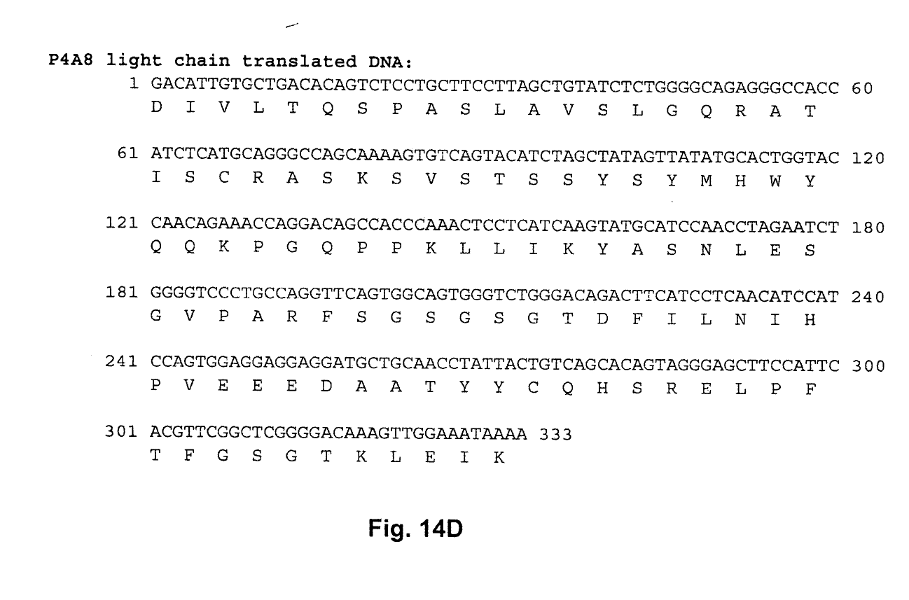

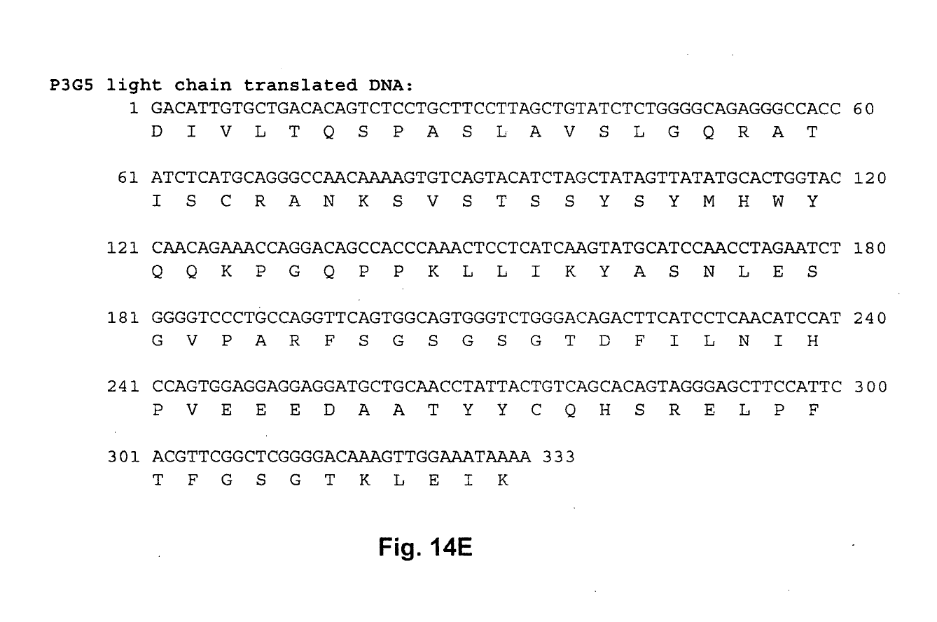

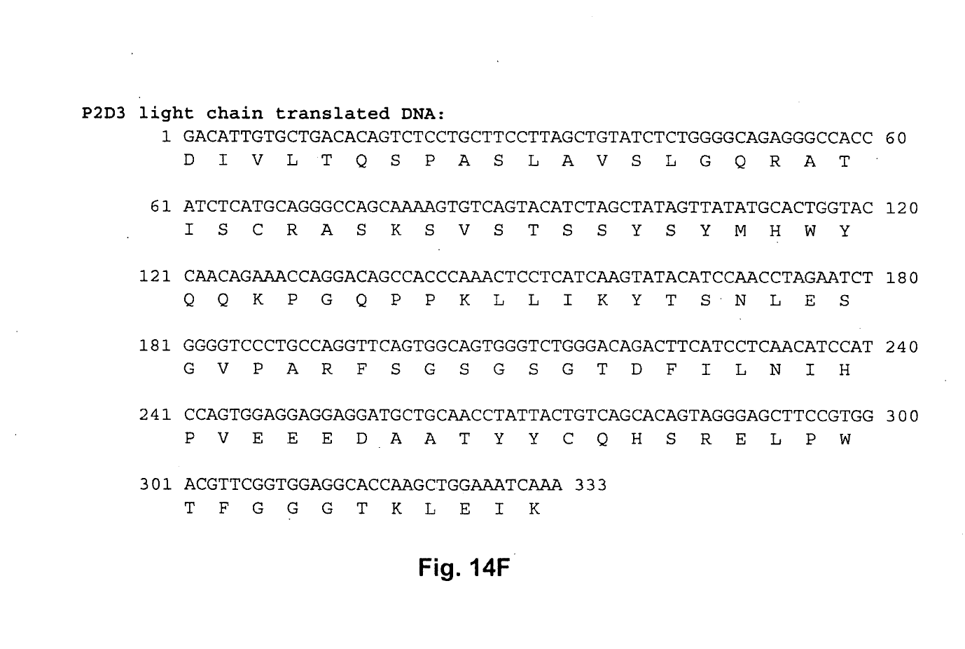

The amino acid sequence of the VH domain of the P4A8 antibody is: QVQLQQSGPEVVRPGVSVKISCKGSGYTFTDYGMHWVKQSHAKSLEWIGVISTYNGYTNYNOKFKGKATMTVDKSSSTAYMELARLTSEDSAIYYCARAYYGNLYYAMDYWGQGTSVTVSS (SEQ ID NO:2). The DNA sequence (SEQ ID NO:17) encoding the VH domain of P4A8 is depicted in FIG. 14A.

The amino acid sequence of the VH domain of the P3G5 antibody is: QVQLQQSGPEVVRPGVSVKISCKGSGYTFTDYGIHWVKQSHAKSLEWIGVISTYNGYTNYNQKFKGKATMTVDKSSSTAYMELARLTSEDSAIYYCARAYYGNLYYAMDYWGQGTSVTVSS (SEQ ID NO:3). The DNA sequence (SEQ ID NO:18) encoding the VH domain of P3G5 is depicted in FIG. 14B.

The amino acid sequence of the VH domain of the P2D3 antibody is: QVSLKESGPGILQPSQTLSLTCSFSGFSLSTSGMGVSWIRQPSGKGLEWLAHIYWDDDKRYNPSLKSRLTISKDTSRNQVFLKITSVDTADTATYYCARRGPDYYGYYPMDYWGQGTSVTVSS (SEQ ID NO:4). The DNA sequence (SEQ ID NO:19) encoding the VH domain of P2D3 is depicted in FIG. 14C.

The amino acid sequence of the VL domain of the P4A8 antibody is: DIVLTQSPASLAVSLGQRATISCRASKSVSTSSYSYMHWYQQKPGQPPKLLIKYASNLESGVPARFSGSGSGTDFILNIHPVEEEDAATYYCQHSRELPFTFGSGTKLEIK (SEQ ID NO:5). The DNA sequence (SEQ ID NO:20) encoding the VL domain of P4A8 is depicted in FIG. 14D.

The amino acid sequence of the VL domain of the P3G5 antibody is: DIVLTQSPASLAVSLGQRATISCRANKSVSTSSYSYMHWYQQKPGQPPKLLIKYASNLESGVPARFSGSGSGTDFILNIHPVEEEDAATYYCQHSRELPFTFGSGTKLEIK (SEQ ID NO:6). The DNA sequence (SEQ ID NO:21) encoding the VL domain of P3G5 is depicted in FIG. 14E.

The amino acid sequence of the VL domain of the P2D3 antibody is: DIVLTQSPASLAVSLGQRATISCRASKSVSTSSYSYMHWYQQKPGQPPKLLIKYTSNLESGVPARFSGSGSGTDFILNIHPVEEEDAATYYCQHSRELPWTFGGGTKLEIK (SEQ ID NO:7). The DNA sequence (SEQ ID NO:22) encoding the VL domain of P2D3 is depicted in FIG. 14F.

The CDRs (CDR-H1/CDR-H2/CDR-H3 and CDR-L1/CDR-L2/CDR-L3) are underlined for each of the variable domain sequences depicted above.

P3D8 has VH and VL domains that are identical to those of P2D3.

An alignment of anti-Fn14 antibody murine heavy chain subgroup II(A) variable domains is as follows:

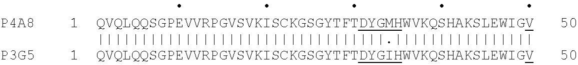

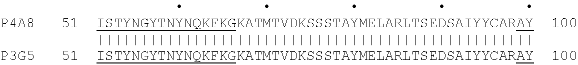

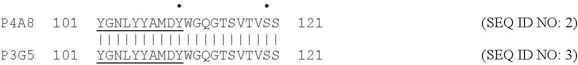

CDR-H1 (left), CDR-H2 (center), and CDR-H3 (right) are underlined for the heavy chains of each of P4A8 and P3G5.

An alignment of anti-Fn14 antibody murine heavy chains P3G5 (IIA) and P2D3 (IB) is as follows:

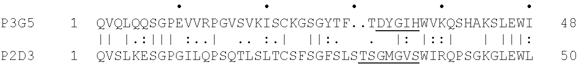

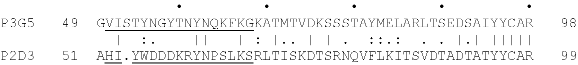

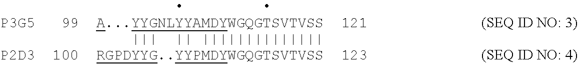

CDR-H1 (left), CDR-H2 (center), and CDR-H3 (right) are underlined for the heavy chains of each of P4A8 and P2D3.

An alignment of anti-Fn14 antibody murine kappa subgroup III light chain variable domains is as follows:

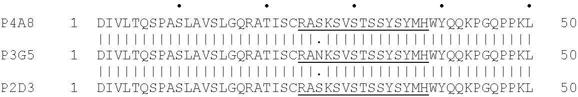

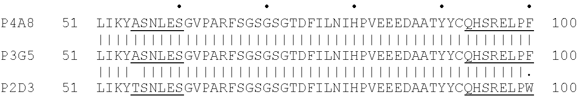

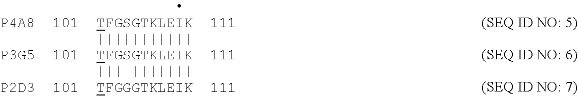

CDR-L1 (left), CDR-L2 (center), and CDR-L3 (right) are underlined for the light chains of each of P4A8, P3G5, and P2D3.

### Example 12

### Chimeric Antibodies

cDNAs encoding the murine P4A8 variable regions of the heavy and light chains were used to construct vectors for expression of murine-human chimeras (chP4A8) in which the muP4A8 variable regions were linked to human IgG1 and kappa constant regions. The sequence of the chimeric P4A8-huIgG1 heavy chain cDNA insert (from the signal sequence's initiator ATG through the terminator TGA) is shown below:

| | | |
|---|---|---|
| 1 | ATGGGATGCA GCTGGGTCAT GCTCTTTCTG GTAGCAACAG CTACAGGTGT | (SEQ ID NO: 32) |
| 51 | GCACTCCCAG GTCCAGCTGC AGCAGTCTGG GCCTGAGGTG GTGAGGCCTG | |
| 101 | GGGTCTCAGT GAAGATTTCC TGCAAGGGTT CCGGCTACAC ATTCACTGAT | |
| 151 | TATGGTATGC ACTGGGTGAA GCAGAGTCAT GCAAAGAGTC TAGAGTGGAT | |
| 201 | TGGAGTTATT AGTACTTACA ATGGTTATAC AAACTACAAC CAGAAGTTTA | |
| 251 | AGGGCAAGGC CACAATGACT GTAGACAAAT CCTCCAGCAC AGCCTATATG | |
| 301 | GAACTTGCCA GATTGACATC TGAGGATTCT GCCATCTATT ACTGTGCAAG | |
| 351 | AGCCTACTAT GGTAACCTTT ACTATGCTAT GGACTACTGG GGTCAAGGAA | |
| 401 | CCTCAGTCAC CGTCTCCTCA GCCTCAACGA AGGGCCCATC GGTCTTCCCC | |
| 451 | CTGGCACCCT CCTCCAAGAG CACCTCTGGG GGCACAGCGG CCCTGGGCTG | |
| 501 | CCTGGTCAAG GACTACTTCC CCGAACCGGT GACGGTGTCG TGGAACTCAG | |
| 551 | GCGCCCTGAC CAGCGGCGTG CACACCTTCC CGGCTGTCCT ACAGTCCTCA | |
| 601 | GGACTCTACT CCCTCAGCAG CGTGGTGACC GTGCCCTCCA GCAGCTTGGG | |
| 651 | CACCCAGACC TACATCTGCA ACGTGAATCA CAAGCCCAGC AACACCAAGG | |
| 701 | TGGACAAGAA AGTTGAGCCC AAATCTTGTG ACAAGACTCA CACATGCCCA | |
| 751 | CCGTGCCCAG CACCTGAACT CCTGGGGGGA CCGTCAGTCT TCCTCTTCCC | |
| 801 | CCCAAAACCC AAGGACACCC TCATGATCTC CCGGACCCCT GAGGTCACAT | |
| 851 | GCGTGGTGGT GGACGTGAGC CACGAAGACC CTGAGGTCAA GTTCAACTGG | |
| 901 | TACGTGGACG GCGTGGAGGT GCATAATGCC AAGACAAAGC CGCGGGAGGA | |
| 951 | GCAGTACAAC AGCACGTACC GTGTGGTCAG CGTCCTCACC GTCCTGCACC | |
| 1001 | AGGACTGGCT GAATGGCAAG GAGTACAAGT GCAAGGTCTC CAACAAAGCC | |
| 1051 | CTCCCAGCCC CCATCGAGAA AACCATCTCC AAAGCCAAAG GGCAGCCCCG | |
| 1101 | AGAACCACAG GTGTACACCC TGCCCCCATC CCGGGATGAG CTGACCAAGA | |
| 1151 | ACCAGGTCAG CCTGACCTGC CTGGTCAAAG GCTTCTATCC CAGCGACATC | |
| 1201 | GCCGTGGAGT GGGAGAGCAA TGGGCAGCCG GAGAACAACT ACAAGACCAC | |
| 1251 | GCCTCCCGTG TTGGACTCCG ACGGCTCCTT CTTCCTCTAC AGCAAGCTCA | |
| 1301 | CCGTGGACAA GAGCAGGTGG CAGCAGGGGA ACGTCTTCTC ATGCTCCGTG | |
| 1351 | ATGCATGAGG CTCTGCACAA CCACTACACG CAGAAGAGCC TCTCCCTGTC | |
| 1401 | TCCCGGTTGA | |

The deduced mature chP4A8 heavy chain protein sequence is shown below:

| | | |
|---|---|---|
| 1 | QVQLQQSGPE VVRPGVSVKI SCKGSGYTFT DYGMHWVKQS HAKSLEWIGV | (SEQ ID NO: 33) |
| 51 | ISTYNGYTNY NQKFKGKATM TVDKSSSTAY MELARLTSED SAIYYCARAY | |
| 101 | YGNLYYAMDY WGQGTSVTVS SASTKGPSVF PLAPSSKSTS GGTAALGCLV | |
| 151 | KDYFPEPVTV SWNSGALTSG VHTFPAVLQS SGLYSLSSVV TVPSSSLGTQ | |
| 201 | TYICNVNHKP SNTKVDKKVE PKSCDKTHTC PPCPAPELLG GPSVFLFPPK | |
| 251 | PKDTLMISRT PEVTCVVVDV SHEDPEVKFN WYVDGVEVHN AKTKPREEQY | |
| 301 | NSTYRVVSVL TVLHQDWLNG KEYKCKVSNK ALPAPIEKTI SKAKGQPREP | |
| 351 | QVYTLPPSRD ELTKNQVSLT CLVKGFYPSD IAVEWESNGQ PENNYKTTPP | |
| 401 | VLDSDGSFFL YSKLTVDKSR WQQGNVFSCS VMHEALHNHY TQKSLSLSPG | |

The sequence of the chimeric P4A8 light chain cDNA insert (from the signal sequence's initiator ATG through the terminator TAG) is shown below:

| | | |
|---|---|---|
| 1 | ATGGAGACAG ACACACTCCT GCTATGGGTA CTGCTGCTCT GGGTTCCAGG | (SEQ ID NO: 34) |
| 51 | TTCCACTGGT GACATTGTGC TGACACAGTC TCCTGCTTCC TTAGCTGTAT | |
| 101 | CTCTGGGGCA GAGGGCCACC ATCTCATGCA GGGCCAGCAA AAGTGTCAGT | |
| 151 | ACATCTAGCT ATAGTTATAT GCACTGGTAC CAACAGAAAC CAGGACAGCC | |
| 201 | ACCCAAACTC CTCATCAAGT ATGCATCCAA CCTAGAATCT GGGGTCCCTG | |
| 251 | CCAGGTTCAG TGGCAGTGGG TCTGGGACAG ACTTCATCCT CAACATCCAT | |
| 301 | CCAGTGGAGG AGGAGGATGC TGCAACCTAT TACTGTCAGC ACAGTAGGGA | |
| 351 | GCTTCCATTC ACGTTCGGCT CGGGGACAAA GTTGGAAATA AAACGTACGG | |
| 401 | TGGCTGCACC ATCTGTCTTC ATCTTCCCGC CATCTGATGA GCAGTTGAAA | |
| 451 | TCTGGAACTG CCTCTGTTGT GTGCCTGCTG AATAACTTCT ATCCCAGAGA | |
| 501 | GGCCAAAGTA CAGTGGAAGG TGGATAACGC CCTCCAATCG GGTAACTCCC | |
| 551 | AGGAGAGTGT CACAGAGCAG GACAGCAAGG ACAGCACCTA CAGCCTCAGC | |
| 601 | AGCACCCTGA CGCTGAGCAA AGCAGACTAC GAGAAACACA AAGTCTACGC | |
| 651 | CTGCGAAGTC ACCCATCAGG GCCTGAGCTC GCCCGTCACA AAGAGCTTCA | |
| 701 | ACAGGGGAGA GTGTTAG | |

The deduced mature chP4A8-human kappa light chain protein sequence is shown below:

| | | |
|---|---|---|
| 1 | DIVLTQSPAS LAVSLGQRAT ISCRASKSVS TSSYSYMHWY QQKPGQPPKL | (SEQ ID NO: 35) |
| 51 | LIKYASNLES GVPARFSGSG SGTDFILNIH PVEEEDAATY YCQHSRELPF | |
| 101 | TFGSGTKLEI KRTVAAPSVF IFPPSDEQLK SGTASVVCLL NNFYPREAKV | |
| 151 | QWKVDNALQS GNSQESVTEQ DSKDSTYSLS STLTLSKADY EKHKVYACEV | |
| 201 | THQGLSSPVT KSFNRGEC | |

Expression vectors (chP4A8 heavy chain vector pXW362 and chP4A8 light chain vector pXW364) were co-transfected into 293-EBNA cells and transfected cells were tested for antibody secretion and specificity (empty vector- and a molecularly cloned irrelevant mAb vector-transfected cells served as controls). Western blot analysis (developed with anti-human heavy and light chain antibodies) of conditioned medium indicated that chP4A8-transfected cells synthesized and efficiently secreted heavy and light chains. Direct FACS binding to human Fn14 confirmed the specificity of chP4A8.

Expression vectors for stable expression of chP4A8 in CHO cells were constructed. A stable CHO cell line secreting chP4A8-huIgG1, kappa mAb was derived by co-transfection with the vectors encoding the light and the heavy chains. The binding affinity of chP4A8 was demonstrated to be equivalent to that of the murine P4A8 mAb by direct binding to surface expressed human Fn14 by dilution titration FACS assay.

### Example 13

### Humanized Antibodies

Examples of two humanized P4A8 (huP4A8) heavy chains (germline huVH1-18 framework/consensus HUMVH1 FR4/P4A8H CDRs) are depicted below (the amino acid and DNA sequences are shown for each; CDRs are underlined and backmutations are shown in bold):

Version H1

(SEQ ID NO: 11)

QVQLVQSGAEVKKPGASVKVSCKGSGYTFTDYGMHWVRQAPGQGLEW
MGVISTYNGYTNYNQKFKGRVTMTVDKSTSTAYMELRSLRSDDTAVYYCA
RAYYGNLYYAMDYWGQGTLVTVSS

(SEQ ID NO: 23)

CAGGTCCAGCTGGTGCAGTCTGGGGCTGAGGTGAAGAAGCCTGGGGC
CTCAGTGAAGGTTTCCTGCAAGGGTTCCGGCTACACATTCACTGATTATG
GCATGCACTGGGTGCGGCAGGCCCCTGGACAAGGGCTAGAGTGGATGGGA
GTTATTAGTACTTACAATGGTTATACAAACTACAACCAGAAGTTTAAGGG
CAGAGTCACAATGACTGTAGACAAATCCACGAGCACAGCCTATATGGAAC
TTCGGAGCTTGAGATCTGACGATACGGCCGTGTATTACTGTGCAAGAGCC
TACTATGGCAACCTTTACTATGCTATGGACTACTGGGGTCAAGGAACCCT
GGTCACCGTCTCCTCA
Version H2

(SEQ ID NO: 12)

QVQLVQSGAEVKKPGASVKVSCKGSGYTFTDYGMHWVRQAPGQGLEW
IGVISTYNGYTNYNQKFKGRATMTVDKSTSTAYMELRSLRSDDTAVYYCA
RAYYGNLYYAMDYWGQGTLVTVSS

(SEQ ID NO: 24)

CAGGTCCAGCTGGTGCAGTCTGGGGCTGAGGTGAAGAAGCCTGGGGC
CTCAGTGAAGGTTTCCTGCAAGGGTTCCGGCTACACATTCACTGATTATG
GCATGCACTGGGTGCGGCAGGCCCCTGGACAAGGGCTCGAGTGGATCGGA
GTTATTAGTACTTACAATGGTTATACAAACTACAACCAGAAGTTTAAGGG
AAGAGCCACAATGACTGTAGACAAATCCACGAGCACAGCCTATATGGAAC
TTCGGAGCTTGAGATCTGACGATACGGCCGTGTATTACTGTGCAAGAGCC
TACTATGGCAACCTTTACTATGCTATGGACTACTGGGGTCAAGGAACCCT
GGTCACCGTCTCCTCA

Examples of three humanized P4A8 (huP4A8) light chains (K037659 framework/P4A8L CDRs) are depicted below (the amino acid and DNA sequences are shown for each; CDRs are underlined and backmutations are shown in bold):

Version L1

(SEQ ID NO: 13)

DIVLTQSPASLAVSLGQRATISCRASKSVSTSSYSYMHWYQQKPGQPPKL
LIKYASNLESGVPARFSGSGSGTDFSLNIHPMEEDDTAMYFCQHSRELPF
TFGGGTKLEIK

(SEQ ID NO: 25)

GACATTGTGCTGACACAGTCTCCTGCTTCCCTGGCTGTATCTCTGGGGC
AGAGGGCCACCATCTCATGCAGGGCCAGCAAAAGTGTCAGTACATCTAGC
TATAGTTATATGCACTGGTACCAACAGAAACCAGGACAGCCACCCAAACT
CCTCATCAAATATGCATCCAACCTAGAATCTGGGGTCCCTGCCAGGTTCA
GTGGCAGTGGGTCTGGGACAGACTTCTCCCTCAACATCCATCCCATGGAG
GAGGACGATACCGCAATGTATTTCTGTCAGCACAGTAGGGAGCTTCCATT
CACGTTCGGCGGAGGGACAAAGTTGGAAATAAAA
Version L2

(SEQ ID NO: 14)

DIVLTQSPASLAVSLGQRATISCRASKSVSTSSYSYMHWYQQKPGQPPKL
LIKYASNLESGVPARFSGSGSGTDFILNIHPMEEDDTAMYFCQHSRELPF
TFGGGTKLEIK

(SEQ ID NO: 26)

GACATTGTGCTGACACAGTCTCCTGCTTCCCTGGCTGTATCTCTGGGGC
AGAGGGCCACCATCTCATGCAGGGCCAGCAAAAGTGTCAGTACATCTAGC
TATAGTTATATGCACTGGTACCAACAGAAACCAGGACAGCCACCCAAACT
CCTCATCAAATATGCATCCAACCTAGAATCTGGGGTCCCTGCCAGGTTCA
GTGGCAGTGGGTCTGGGACAGACTTCATCCTCAACATCCATCCAATGGAG
GAGGACGATACCGCAATGTATTTCTGTCAGCACAGTAGGGAGCTTCCATT
CACGTTCGGCGGAGGGACAAAGTTGGAAATAAAA
Version L3

(SEQ ID NO: 15)

DIVLTQSPASLAVSLGQRATISCRASKSVSTSSYSYMHWYQQKPGQPPKL
LIKYASNLESGVPARFSGSGSGTDFILNIHPMEEDDTATYYCQHSRELPF
TFGGGTKLEIK

(SEQ ID NO: 27)

GACATTGTGCTGACACAGTCTCCTGCTTCCCTGGCTGTATCTCTGGGGC
AGAGGGCCACCATCTCATGCAGGGCCAGCAAAAGTGTCAGTACATCTAGC
TATAGTTATATGCACTGGTACCAACAGAAACCAGGACAGCCACCCAAACT
CCTCATCAAATATGCATCCAACCTAGAATCTGGGGTCCCTGCCAGGTTCA
GTGGCAGTGGGTCTGGGACAGACTTCATCCTCAACATCCATCCAATGGAG
GAGGACGATACCGCAACCTATTACTGTCAACACAGTAGGGAGCTTCCATT
CACGTTCGGCGGAGGGACAAAGTTGGAAATAAAA

A stable CHO expression vector for the H1 huP4A8-huIgG1 heavy chain, pYL310, was constructed. The sequence of the H1 huP4A8-huIgG1 heavy chain cDNA insert of pYL310 (from the signal sequence's initiator ATG through the terminator TGA) is shown below:

| | | |
|---|---|---|
| 1 | ATGGGATGCA GCTGGGTCAT GCTCTTTCTG GTAGCAACAG CTACAGGCGT | (SEQ ID NO: 36) |
| 51 | GCACTCCCAG GTCCAGCTGG TGCAGTCTGG GGCTGAGGTG AAGAAGCCTG | |
| 101 | GGGCCTCAGT GAAGGTTTCC TGCAAGGGTT CCGGCTACAC ATTCACTGAT | |
| 151 | TATGGCATGC ACTGGGTGCG GCAGGCCCCT GGACAAGGGC TAGAGTGGAT | |
| 201 | GGGAGTTATT AGTACTTACA ATGGTTATAC AAACTACAAC CAGAAGTTTA | |
| 251 | AGGGCAGAGT CACAATGACT GTAGACAAAT CCACGAGCAC AGCCTATATG | |
| 301 | GAACTTCGGA GCTTGAGATC TGACGATACG GCCGTGTATT ACTGTGCAAG | |
| 351 | AGCCTACTAT GGCAACCTTT ACTATGCTAT GGACTACTGG GGTCAAGGAA | |
| 401 | CCCTGGTCAC CGTCTCCTCA GCCTCCACCA AGGGCCCATC GGTCTTCCCC | |
| 451 | CTGGCACCCT CCTCCAAGAG CACCTCTGGG GGCACAGCGG CCCTGGGCTG | |
| 501 | CCTGGTCAAG GACTACTTCC CCGAACCGGT GACGGTGTCG TGGAACTCAG | |
| 551 | GCGCCCTGAC CAGCGGCGTG CACACCTTCC CGGCTGTCCT ACAGTCCTCA | |
| 601 | GGACTCTACT CCCTCAGCAG CGTGGTGACC GTGCCCTCCA GCAGCTTGGG | |
| 651 | CACCCAGACC TACATCTGCA ACGTGAATCA CAAGCCCAGC AACACCAAGG | |
| 701 | TGGACAAGAA AGTTGAGCCC AAATCTTGTG ACAAGACTCA CACATGCCCA | |
| 751 | CCGTGCCCAG CACCTGAACT CCTGGGGGGA CCGTCAGTCT TCCTCTTCCC | |
| 801 | CCCAAAACCC AAGGACACCC TCATGATCTC CCGGACCCCT GAGGTCACAT | |
| 851 | GCGTGGTGGT GGACGTGAGC CACGAAGACC CTGAGGTCAA GTTCAACTGG | |
| 901 | TACGTGGACG GCGTGGAGGT GCATAATGCC AAGACAAAGC CGCGGGAGGA | |
| 951 | GCAGTACAAC AGCACGTACC GTGTGGTCAG CGTCCTCACC GTCCTGCACC | |
| 1001 | AGGACTGGCT GAATGGCAAG GAGTACAAGT GCAAGGTCTC CAACAAAGCC | |
| 1051 | CTCCCAGCCC CCATCGAGAA AACCATCTCC AAAGCCAAAG GGCAGCCCCG | |
| 1101 | AGAACCACAG GTGTACACCC TGCCCCCATC CCGGGATGAG CTGACCAAGA | |
| 1151 | ACCAGGTCAG CCTGACCTGC CTGGTCAAAG GCTTCTATCC CAGCGACATC | |
| 1201 | GCCGTGGAGT GGGAGAGCAA TGGGCAGCCG GAGAACAACT ACAAGACCAC | |
| 1251 | GCCTCCCGTG TTGGACTCCG ACGGCTCCTT CTTCCTCTAC AGCAAGCTCA | |
| 1301 | CCGTGGACAA GAGCAGGTGG CAGCAGGGGA ACGTCTTCTC ATGCTCCGTG | |
| 1351 | ATGCATGAGG CTCTGCACAA CCACTACACG CAGAAGAGCC TCTCCCTGTC | |
| 1401 | TCCCGGTTGA | |

The deduced mature huP4A8-IgG1 H1 heavy chain protein sequence encoded by pYL310 is shown below:

| | | |
|---|---|---|
| 1 | QVQLVQSGAE VKKPGASVKV SCKGSGYTFT DYGMHWVRQA PGQGLEWMGV | (SEQ ID NO: 37) |
| 51 | ISTYNGYTNY NQKFKGRVTM TVDKSTSTAY MELRSLRSDD TAVYYCARAY | |
| 101 | YGNLYYAMDY WGQGTLVTVS SASTKGPSVF PLAPSSKSTS GGTAALGCLV | |
| 151 | KDYFPEPVTV SWNSGALTSG VHTFPAVLQS SGLYSLSSVV TVPSSSLGTQ | |
| 201 | TYICNVNHKP SNTKVDKKVE PKSCDKTHTC PPCPAPELLG GPSVFLFPPK | |
| 251 | PKDTLMISRT PEVTCVVVDV SHEDPEVKFN WYVDGVEVHN AKTKPREEQY | |
| 301 | NSTYRVVSVL TVLHQDWLNG KEYKCKVSNK ALPAPIEKTI SKAKGQPREP | |
| 351 | QVYTLPPSRD ELTKNQVSLT CLVKGFYPSD IAVEWESNGQ PENNYKTTPP | |
| 401 | VLDSDGSFFL YSKLTVDKSR WQQGNVFSCS VMHEALHNHY TQKSLSLSPG | |

A stable CHO expression vector for the H2 huP4A8-huIgG1 heavy chain, pYL320, was constructed. The sequence of the H2 huP4A8-huIgG1 heavy chain cDNA insert of pYL320 (from the signal sequence's initiator ATG through the terminator TGA) is shown below:

| | | |
|---|---|---|
| 1 | ATGGGATGCA GCTGGGTCAT GCTCTTTCTG GTAGCAACAG CTACAGGCGT | (SEQ ID NO: 38) |
| 51 | GCACTCCCAG GTCCAGCTGG TGCAGTCTGG GGCTGAGGTG AAGAAGCCTG | |
| 101 | GGGCCTCAGT GAAGGTTTCC TGCAAGGGTT CCGGCTACAC ATTCACTGAT | |
| 151 | TATGGCATGC ACTGGGTGCG GCAGGCCCCT GGACAAGGGC TCGAGTGGAT | |
| 201 | CGGAGTTATT AGTACTTACA ATGGTTATAC AAACTACAAC CAGAAGTTTA | |
| 251 | AGGGAAGAGC CACAATGACT GTAGACAAAT CCACGAGCAC AGCCTATATG | |
| 301 | GAACTTCGGA GCTTGAGATC TGACGATACG GCCGTGTATT ACTGTGCAAG | |
| 351 | AGCCTACTAT GGCAACCTTT ACTATGCTAT GGACTACTGG GGTCAAGGAA | |
| 401 | CCCTGGTCAC CGTCTCCTCA GCCTCCACCA AGGGCCCATC GGTCTTCCCC | |
| 451 | CTGGCACCCT CCTCCAAGAG CACCTCTGGG GGCACAGCGG CCCTGGGCTG | |
| 501 | CCTGGTCAAG GACTACTTCC CCGAACCGGT GACGGTGTCG TGGAACTCAG | |
| 551 | GCGCCCTGAC CAGCGGCGTG CACACCTTCC CGGCTGTCCT ACAGTCCTCA | |
| 601 | GGACTCTACT CCCTCAGCAG CGTGGTGACC GTGCCCTCCA GCAGCTTGGG | |
| 651 | CACCCAGACC TACATCTGCA ACGTGAATCA CAAGCCCAGC AACACCAAGG | |
| 701 | TGGACAAGAA AGTTGAGCCC AAATCTTGTG ACAAGACTCA CACATGCCCA | |
| 751 | CCGTGCCCAG CACCTGAACT CCTGGGGGGA CCGTCAGTCT TCCTCTTCCC | |
| 801 | CCCAAAACCC AAGGACACCC TCATGATCTC CCGGACCCCT GAGGTCACAT | |
| 851 | GCGTGGTGGT GGACGTGAGC CACGAAGACC CTGAGGTCAA GTTCAACTGG | |
| 901 | TACGTGGACG GCGTGGAGGT GCATAATGCC AAGACAAAGC CGCGGGAGGA | |
| 951 | GCAGTACAAC AGCACGTACC GTGTGGTCAG CGTCCTCACC GTCCTGCACC | |
| 1001 | AGGACTGGCT GAATGGCAAG GAGTACAAGT GCAAGGTCTC CAACAAAGCC | |
| 1051 | CTCCCAGCCC CCATCGAGAA AACCATCTCC AAAGCCAAAG GGCAGCCCCG | |
| 1101 | AGAACCACAG GTGTACACCC TGCCCCCATC CCGGGATGAG CTGACCAAGA | |
| 1151 | ACCAGGTCAG CCTGACCTGC CTGGTCAAAG GCTTCTATCC CAGCGACATC | |
| 1201 | GCCGTGGAGT GGGAGAGCAA TGGGCAGCCG GAGAACAACT ACAAGACCAC | |
| 1251 | GCCTCCCGTG TTGGACTCCG ACGGCTCCTT CTTCCTCTAC AGCAAGCTCA | |
| 1301 | CCGTGGACAA GAGCAGGTGG CAGCAGGGGA ACGTCTTCTC ATGCTCCGTG | |
| 1351 | ATGCATGAGG CTCTGCACAA CCACTACACG CAGAAGAGCC TCTCCCTGTC | |
| 1401 | TCCCGGTTGA | |

The deduced mature huP4A8-IgG1 H2 heavy chain protein sequence encoded by pYL320 is shown below:

| | | |
|---|---|---|
| 1 | QVQLVQSGAE VKKPGASVKV SCKGSGYTFT DYGMHWVRQA PGQGLEWIGV | (SEQ ID NO: 39) |
| 51 | ISTYNGYTNY NQKFKGRATM TVDKSTSTAY MELRSLRSDD TAVYYCARAY | |
| 101 | YGNLYYAMDY WGQGTLVTVS SASTKGPSVF PLAPSSKSTS GGTAALGCLV | |
| 151 | KDYFPEPVTV SWNSGALTSG VHTFPAVLQS SGLYSLSSVV TVPSSSLGTQ | |
| 201 | TYICNVNHKP SNTKVDKKVE PKSCDKTHTC PPCPAPELLG GPSVFLFPPK | |
| 251 | PKDTLMISRT PEVTCVVVDV SHEDPEVKFN WYVDGVEVHN AKTKPREEQY | |
| 301 | NSTYRVVSVL TVLHQDWLNG KEYKCKVSNK ALPAPIEKTI SKAKGQPREP | |
| 351 | QVYTLPPSRD ELTKNQVSLT CLVKGFYPSD IAVEWESNGQ PENNYKTTPP | |
| 401 | VLDSDGSFFL YSKLTVDKSR WQQGNVFSCS VMHEALHNHY TQKSLSLSPG | |

A stable CHO expression vector for the full-length version L2 huP4A8-kappa light chain, pYL317, cDNA was also constructed. The sequence of the huP4A8 L2 kappa light chain cDNA insert of pYL317 (from the signal sequence's initiator ATG through the terminator TAG) is shown below:

| | | |
|---|---|---|
| 1 | ATGGAGACAG ACACACTCCT GCTATGGGTA CTGCTGCTCT GGGTTCCTGG | (SEQ ID NO: 40) |
| 51 | TTCCACTGGT GACATTGTGC TGACACAGTC TCCTGCTTCC CTGGCTGTAT | |
| 101 | CTCTGGGGCA GAGGGCCACC ATCTCATGCA GGGCCAGCAA AAGTGTCAGT | |
| 151 | ACATCTAGCT ATAGTTATAT GCACTGGTAC CAACAGAAAC CAGGACAGCC | |
| 201 | ACCCAAACTC CTCATCAAAT ATGCATCCAA CCTAGAATCT GGGGTCCCTG | |
| 251 | CCAGGTTCAG TGGCAGTGGG TCTGGGACAG ACTTCATCCT CAACATCCAT | |
| 301 | CCAATGGAGG AGGACGATAC CGCAATGTAT TTCTGTCAGC ACAGTAGGGA | |
| 351 | GCTTCCATTC ACGTTCGGCG GAGGGACAAA GTTGGAAATA AAACGTACGG | |
| 401 | TGGCTGCACC ATCTGTCTTC ATCTTCCCGC CATCTGATGA GCAGTTGAAA | |
| 451 | TCTGGAACTG CCTCTGTTGT GTGCCTGCTG AATAACTTCT ATCCCAGAGA | |
| 501 | GGCCAAAGTA CAGTGGAAGG TGGATAACGC CCTCCAATCG GGTAACTCCC | |
| 551 | AGGAGAGTGT CACAGAGCAG GACAGCAAGG ACAGCACCTA CAGCCTCAGC | |
| 601 | AGCACCCTGA CGCTGAGCAA AGCAGACTAC GAGAAACACA AAGTCTACGC | |
| 651 | CTGCGAAGTC ACCCATCAGG GCCTGAGCTC GCCCGTCACA AAGAGCTTCA | |
| 701 | ACAGGGGAGA GTGTTAG | |

The deduced mature huP4A8 L2 kappa light chain protein sequence encoded by pYL317 is shown below:

| | | |
|---|---|---|
| 1 | DIVLTQSPAS LAVSLGQRAT ISCRASKSVS TSSYSYMHWY QQKPGQPPKL | (SEQ ID NO: 41) |
| 51 | LIKYASNLES GVPARFSGSG SGTDFILNIH PMEEDDTAMY FCQHSRELPF | |
| 101 | TFGGGTKLEI KRTVAAPSVF IFPPSDEQLK SGTASVVCLL NNFYPREAKV | |
| 151 | QWKVDNALQS GNSQESVTEQ DSKDSTYSLS STLTLSKADY EKHKVYACEV | |
| 201 | THQGLSSPVT KSFNRGEC | |

A stable CHO expression vector for the full-length version L1 huP4A8-kappa light chain cDNA variant, pYL321, was also constructed. The sequence of the huP4A8 L1 kappa light chain cDNA insert of pYL321 (from the signal sequence's initiator ATG through the terminator TAG) is shown below:

| | | |
|---|---|---|
| 1 | ATGGAGACAG ACACACTCCT GCTATGGGTA CTGCTGCTCT GGGTTCCTGG | (SEQ ID NO: 42) |
| 51 | TTCCACTGGT GACATTGTGC TGACACAGTC TCCTGCTTCC CTGGCTGTAT | |
| 101 | CTCTGGGGCA GAGGGCCACC ATCTCATGCA GGGCCAGCAA AAGTGTCAGT | |
| 151 | ACATCTAGCT ATAGTTATAT GCACTGGTAC CAACAGAAAC CAGGACAGCC | |
| 201 | ACCCAAACTC CTCATCAAAT ATGCATCCAA CCTAGAATCT GGGGTCCCTG | |
| 251 | CCAGGTTCAG TGGCAGTGGG TCTGGGACAG ACTTCTCCCT CAACATCCAT | |
| 301 | CCCATGGAGG AGGACGATAC CGCAATGTAT TTCTGTCAGC ACAGTAGGGA | |
| 351 | GCTTCCATTC ACGTTCGGCG GAGGGACAAA GTTGGAAATA AAACGTACGG | |
| 401 | TGGCTGCACC ATCTGTCTTC ATCTTCCCGC CATCTGATGA GCAGTTGAAA | |
| 451 | TCTGGAACTG CCTCTGTTGT GTGCCTGCTG AATAACTTCT ATCCCAGAGA | |
| 501 | GGCCAAAGTA CAGTGGAAGG TGGATAACGC CCTCCAATCG GGTAACTCCC | |
| 551 | AGGAGAGTGT CACAGAGCAG GACAGCAAGG ACAGCACCTA CAGCCTCAGC | |
| 601 | AGCACCCTGA CGCTGAGCAA AGCAGACTAC GAGAAACACA AAGTCTACGC | |
| 651 | CTGCGAAGTC ACCCATCAGG GCCTGAGCTC GCCCGTCACA AAGAGCTTCA | |
| 701 | ACAGGGGAGA GTGTTAG | |

The deduced mature huP4A8 L1 kappa light chain protein sequence encoded by pYL321 is shown below:

| | | |
|---|---|---|
| 1 | DIVLTQSPAS LAVSLGQRAT ISCRASKSVS TSSYSYMHWY QQKPGQPPKL | (SEQ ID NO: 43) |
| 51 | LIKYASNLES GVPARFSGSG SGTDFSLNIH PMEEDDTAMY FCQHSRELPF | |
| 101 | TFGGGTKLEI KRTVAAPSVF IFPPSDEQLK SGTASVVCLL NNFYPREAKV | |
| 151 | QWKVDNALQS GNSQESVTEQ DSKDSTYSLS STLTLSKADY EKHKVYACEV | |
| 201 | THQGLSSPVT KSFNRGEC | |

A stable CHO expression vector for the full-length version L3 huP4A8-kappa light chain cDNA variant, pYL322, was constructed. The sequence of the huP4A8 L3 kappa light chain cDNA insert of pYL322 (from the signal sequence's initiator ATG through the terminator TAG) is shown below:

| | | |
|---|---|---|
| 1 | ATGGAGACAG ACACACTCCT GCTATGGGTA CTGCTGCTCT GGGTTCCTGG | (SEQ ID NO: 44) |
| 51 | TTCCACTGGT GACATTGTGC TGACACAGTC TCCTGCTTCC CTGGCTGTAT | |
| 101 | CTCTGGGGCA GAGGGCCACC ATCTCATGCA GGGCCAGCAA AAGTGTCAGT | |
| 151 | ACATCTAGCT ATAGTTATAT GCACTGGTAC CAACAGAAAC CAGGACAGCC | |
| 201 | ACCCAAACTC CTCATCAAAT ATGCATCCAA CCTAGAATCT GGGGTCCCTG | |
| 251 | CCAGGTTCAG TGGCAGTGGG TCTGGGACAG ACTTCATCCT CAACATCCAT | |
| 301 | CCAATGGAGG AGGACGATAC CGCAACCTAT TACTGTCAAC ACAGTAGGGA | |
| 351 | GCTTCCATTC ACGTTCGGCG GAGGGACAAA GTTGGAAATA AAACGTACGG | |
| 401 | TGGCTGCACC ATCTGTCTTC ATCTTCCCGC CATCTGATGA GCAGTTGAAA | |
| 451 | TCTGGAACTG CCTCTGTTGT GTGCCTGCTG AATAACTTCT ATCCCAGAGA | |
| 501 | GGCCAAAGTA CAGTGGAAGG TGGATAACGC CCTCCAATCG GGTAACTCCC | |
| 551 | AGGAGAGTGT CACAGAGCAG GACAGCAAGG ACAGCACCTA CAGCCTCAGC | |
| 601 | AGCACCCTGA CGCTGAGCAA AGCAGACTAC GAGAAACACA AAGTCTACGC | |
| 651 | CTGCGAAGTC ACCCATCAGG GCCTGAGCTC GCCCGTCACA AAGAGCTTCA | |
| 701 | ACAGGGGAGA GTGTTAG | |

The deduced mature huP4A8 L3 kappa light chain protein sequence encoded by pYL322 is shown below:

| | | |
|---|---|---|
| 1 | DIVLTQSPAS LAVSLGQRAT ISCRASKSVS TSSYSYMHWY QQKPGQPPKL | (SEQ ID NO: 45) |
| 51 | LIKYASNLES GVPARFSGSG SGTDFILNIH PMEEDDTATY YCQHSRELPF | |
| 101 | TFGGGTKLEI KRTVAAPSVF IFPPSDEQLK SGTASVVCLL NNFYPREAKV | |
| 151 | QWKVDNALQS GNSQESVTEQ DSKDSTYSLS STLTLSKADY EKHKVYACEV | |
| 201 | THQGLSSPVT KSFNRGEC | |

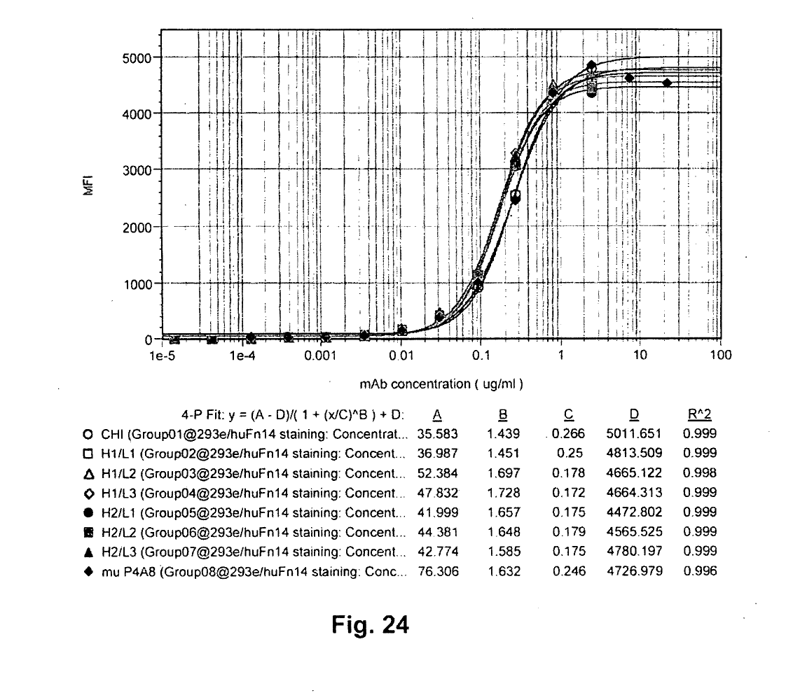

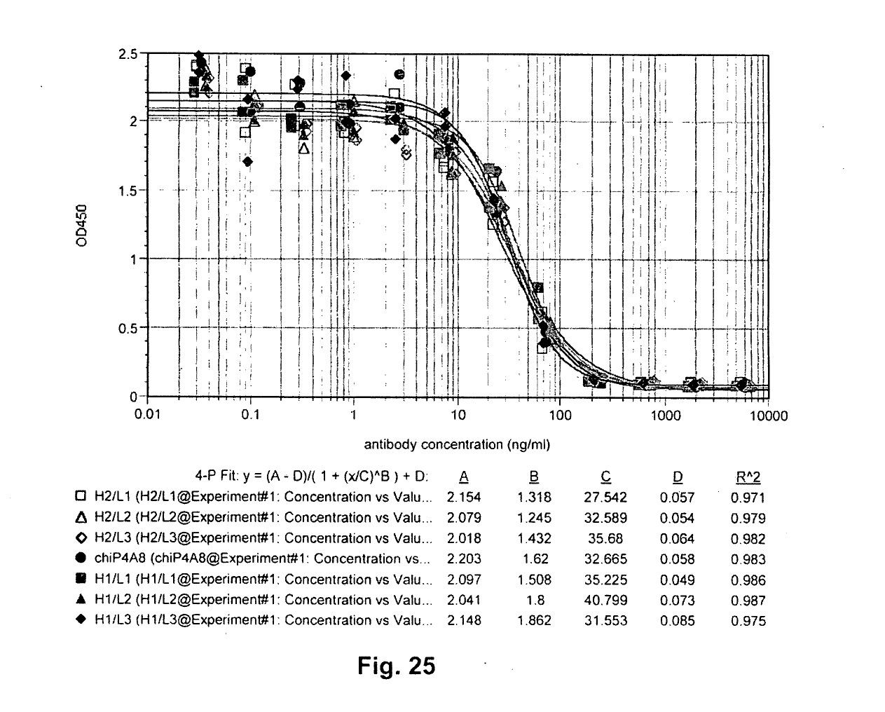

All six versions of huP4A8 were expressed transiently in 293E cells by co-transfection of heavy chain and light chain plasmids. All versions of huP4A8 were assembled and secreted at similar titers (titers in conditioned medium from transiently transfected cells were quantitated by ELISA and normalized for binding assays). FIG. 24 shows that all versions of huP4A8 expressed transiently had equivalent bioactivities to chP4A8 as assayed by FACS dilution titration direct binding to surface human Fn14 transiently overexpressed in 293E cells. FIG. 25 shows that all six versions of huP4A8 retained Fn14 binding affinities essentially equivalent to chP4A8 assayed by competition ELISA (binding to huFn14-huFc fusion protein coated onto the wells of a 96 well plate, competing with binding by a constant amount of biotinylated murine P4A8). A stable CHO cell line secreting huP4A8-huIgG1, kappa (H1/L1) mAb was derived by co-transfection with pYL310 and pYL321. This antibody has a glycosylation at Asn301 (natural glycosylation site in CH2 domain of IgG1) in the mature sequence of the heavy chain. Asn301 corresponds to Asn297 in the Kabat EU numbering scheme (see Kabat et al., 1991, “Sequences of proteins of immunological interest,” NIH publication No. 91-3242).

A humanized version of P4A8 was constructed that contains the H1/L1 combination above and has an aglycosylated S228P/T299A huIgG4 heavy chain (huP4A8-aglyG4P heavy chain). The IgG4 heavy chain S228P change is made to eliminate half-antibody and the T299A change is made to eliminate the CH₂'s N-linked glycan and thereby attenuate effector function. The aglycosylated antibody exhibits reduced effector function with respect to both antibody-dependent cellular cytotoxicity (ADCC) and complement-mediated cytotoxicity (CMC). The mature sequence of the heavy chain (SEQ ID NO:8) is depicted below, with residues S228P and T299A underlined and in bold (the VH domain corresponds to residues I-121; the IgG4 constant domain corresponds to residues 122-447):

| | | |
|---|---|---|
| 1 | QVQLVQSGAE VKKPGASVKV SCKGSGYTFT DYGMHWVRQA PGQGLEWMGV | (SEQ ID NO: 8) |
| 51 | ISTYNGYTNY NQKFKGRVTM TVDKSTSTAY MELRSLRSDD TAVYYCARAY | |
| 101 | YGNLYYAMDY WGQGTLVTVS SASTKGPSVF PLAPCSRSTS ESTAALGCLV | |
| 151 | KDYFPEPVTV SWNSGALTSG VHTFPAVLQS SGLYSLSSVV TVPSSSLGTK | |
| 201 | TYTCNVDHKP SNTKVDKRVE SKYGPPCPPC PAPEFLGGPS VFLFPPKPKD | |
| 251 | TLMISRTPEV TCVVVDVSQE DPEVQFNWYV DGVEVHNAKT KPREEQFNSA | |
| 301 | YRVVSVLTVL HQDWLNGKEY KCKVSNKGLP SSIEKTISKA KGQPREPQVY | |
| 351 | TLPPSQEEMT KNQVSLTCLV KGFYPSDIAV EWESNGQPEN NYKTTPPVLD | |
| 401 | SDGSFFLYSR LTVDKSRWQE GNVFSCSVMH EALHNHYTQK SLSLSLG | |

This protein is encoded by the following nucleotide sequence:

| | | |
|---|---|---|
| 1 | ATGGGATGCA GCTGGGTCAT GCTCTTTCTG GTAGCAACAG CTACAGGCGT | (SEQ ID NO: 46) |
| 51 | GCACTCCCAG GTCCAGCTGG TGCAGTCTGG GGCTGAGGTG AAGAAGCCTG | |
| 101 | GGGCCTCAGT GAAGGTTTCC TGCAAGGGTT CCGGCTACAC ATTCACTGAT | |
| 151 | TATGGCATGC ACTGGGTGCG GCAGGCCCCT GGACAAGGGC TAGAGTGGAT | |
| 201 | GGGAGTTATT AGTACTTACA ATGGTTATAC AAACTACAAC CAGAAGTTTA | |
| 251 | AGGGCAGAGT CACAATGACT GTAGACAAAT CCACGAGCAC AGCCTATATG | |
| 301 | GAACTTCGGA GCTTGAGATC TGACGATACG GCCGTGTATT ACTGTGCAAG | |
| 351 | AGCCTACTAT GGCAACCTTT ACTATGCTAT GGACTACTGG GGTCAAGGAA | |
| 401 | CCCTGGTCAC CGTCTCCTCA GCCTCCACCA AGGGCCCATC CGTCTTCCCC | |
| 451 | CTGGCGCCCT GCTCCAGATC TACCTCCGAG AGCACAGCCG CCCTGGGCTG | |
| 501 | CCTGGTCAAG GACTACTTCC CCGAACCGGT GACGGTGTCG TGGAACTCAG | |
| 551 | GCGCCCTGAC CAGCGGCGTG CACACCTTCC CGGCTGTCCT ACAGTCCTCA | |
| 601 | GGACTCTACT CCCTCAGCAG CGTGGTGACC GTGCCCTCCA GCAGCTTGGG | |
| 651 | CACGAAGACC TACACCTGCA ACGTAGATCA CAAGCCCAGC AACACCAAGG | |
| 701 | TGGACAAGAG AGTTGAGTCC AAATATGGTC CCCCATGCCC ACCGTGCCCA | |
| 751 | GCACCTGAGT TCCTGGGGGG ACCATCAGTC TTCCTGTTCC CCCCAAAACC | |
| 801 | CAAGGACACT CTCATGATCT CCCGGACCCC TGAGGTCACG TGCGTGGTGG | |
| 851 | TGGACGTGAG CCAGGAAGAC CCCGAGGTCC AGTTCAACTG GTACGTGGAT | |
| 901 | GGCGTGGAGG TGCATAATGC CAAGACAAAG CCGCGGGAGG AGCAGTTCAA | |
| 951 | CAGCGCGTAC CGTGTGGTCA GCGTCCTCAC CGTCCTGCAC CAGGACTGGC | |
| 1001 | TGAACGGCAA GGAGTACAAG TGCAAGGTCT CCAACAAAGG CCTCCCGTCC | |
| 1051 | TCCATCGAGA AAACCATCTC CAAAGCCAAA GGGCAGCCCC GAGAGCCACA | |
| 1101 | AGTGTACACC CTGCCCCCAT CCCAGGAGGA GATGACCAAG AACCAGGTCA | |
| 1151 | GCCTGACCTG CCTGGTCAAA GGCTTCTACC CCAGCGACAT CGCCGTGGAG | |
| 1201 | TGGGAGAGCA ATGGGCAGCC GGAGAACAAC TACAAGACCA CGCCTCCCGT | |
| 1251 | CCTCGATTCC GACGGCTCCT TCTTCCTCTA CAGCAGGCTA ACCGTGGACA | |
| 1301 | AGAGCAGGTG GCAGGAGGGG AATGTCTTCT CATGCTCCGT GATGCATGAG | |
| 1351 | GCTCTGCACA ACCACTACAC ACAGAAGAGC CTCTCCCTGT CTCTGGGTTG | |
| 1401 | A | |

The mature sequence of the huP4A8 kappa light chain (SEQ ID NO:9) of the antibody is as follows (the VL domain corresponds to residues 1-111):

| | | |
|---|---|---|
| 1 | DIVLTQSPAS LAVSLGQRAT ISCRASKSVS TSSYSYMHWY QQKPGQPPKL | (SEQ ID NO: 9) |
| 51 | LIKYASNLES GVPARFSGSG SGTDFSLNIH PMEEDDTAMY FCQHSRELPF | |
| 101 | TFGGGTKLEI KRTVAAPSVF IFPPSDEQLK SGTASVVCLL NNFYPREAKV | |
| 151 | QWKVDNALQS GNSQESVTEQ DSKDSTYSLS STLTLSKADY EKHKVYACEV | |
| 201 | THQGLSSPVT KSFNRGEC | |

In addition to the aglycosylated huIgG4 heavy chain above, a T299A aglycosylated huP4A8-huIgG1 heavy chain can also be used in combination with the light chain of SEQ ID NO:9. The mature sequence of the T299A aglycosylated huP4A8-huIgG1 heavy chain (SEQ ID NO:16), with residue T299A underlined and in bold, is depicted below (the VH domain corresponds to residues 1-121):

| | | |
|---|---|---|
| 1 | QVQLVQSGAE VKKPGASVKV SCKGSGYTFT DYGMHWVRQA PGQGLEWMGV | (SEQ ID NO: 16) |
| 51 | ISTYNGYTNY NQKFKGRVTM TVDKSTSTAY MELRSLRSDD TAVYYCARAY | |
| 101 | YGNLYYAMDY WGQGTLVTVS SASTKGPSVF PLAPSSKSTS GGTAALGCLV | |
| 151 | KDYFPEPVTV SWNSGALTSG VHTFPAVLQS SGLYSLSSVV TVPSSSLGTQ | |
| 201 | TYICNVNHKP SNTKVDKKVE PKSCDKTHTC PPCPAPELLG GPSVFLFPPK | |
| 251 | PKDTLMISRT PEVTCVVVDV SHEDPEVKFN WYVDGVEVHN AKTKPREEQY | |
| 301 | NSAYRVVSVL TVLHQDWLNG KEYKCKVSNK ALPAPIEKTI SKAKGQPREP | |
| 351 | QVYTLPPSRD ELTKNQVSLT CLVKGFYPSD IAVEWESNGQ PENNYKTTPP | |
| 401 | VLDSDGSFFL YSKLTVDKSR WQQGNVFSCS VMHEALHNHY TQKSLSLSPG | |

This protein is encoded by the following nucleotide sequence:

| | | |
|---|---|---|
| 1 | ATGGGATGCA GCTGGGTCAT GCTCTTTCTG GTAGCAACAG CTACAGGCGT | (SEQ ID NO: 47) |
| 51 | GCACTCCCAG GTCCAGCTGG TGCAGTCTGG GGCTGAGGTG AAGAAGCCTG | |
| 101 | GGGCCTCAGT GAAGGTTTCC TGCAAGGGTT CCGGCTACAC ATTCACTGAT | |
| 151 | TATGGCATGC ACTGGGTGCG GCAGGCCCCT GGACAAGGGC TAGAGTGGAT | |
| 201 | GGGAGTTATT AGTACTTACA ATGGTTATAC AAACTACAAC CAGAAGTTTA | |
| 251 | AGGGCAGAGT CACAATGACT GTAGACAAAT CCACGAGCAC AGCCTATATG | |
| 301 | CAACTTCGGA GCTTGAGATC TGACGATACG GCCGTGTATT ACTGTGCAAG | |
| 351 | AGCCTACTAT GGCAACCTTT ACTATGCTAT GGACTACTGG GGTCAAGGAA | |
| 401 | CCCTGGTCAC CGTCTCCTCA GCCTCCACCA AGGGCCCATC GGTCTTCCCC | |
| 451 | CTGGCACCCT CCTCCAAGAG CACCTCTGGG GGCACAGCGG CCCTGGGCTG | |
| 501 | CCTGGTCAAG GACTACTTCC CCGAACCGGT GACGGTGTCG TGGAACTCAG | |
| 551 | GCGCCCTGAC CAGCGGCGTG CACACCTTCC CGGCTGTCCT ACAGTCCTCA | |
| 601 | GGACTCTACT CCCTCAGCAG CGTGGTGACC GTGCCCTCCA GCAGCTTGGG | |
| 651 | CACCCAGACC TACATCTGCA ACGTGAATCA CAAGCCCAGC AACACCAAGG | |
| 701 | TGGACAAGAA AGTTGAGCCC AAATCTTGTG ACAAGACTCA CACATGCCCA | |
| 751 | CCGTGCCCAG CACCTGAACT CCTGGGGGGA CCGTCAGTCT TCCTCTTCCC | |
| 801 | CCCAAAACCC AAGGACACCC TCATGATCTC CCGGACCCCT GAGGTCACAT | |
| 851 | GCGTGGTGGT GGACGTGAGC CACGAAGACC CTGAGGTCAA GTTCAACTGG | |
| 901 | TACGTGGACG GCGTGGAGGT GCATAATGCC AAGACAAAGC CGCGGGAGGA | |
| 951 | GCAGTACAAC AGCGCGTACC GTGTGGTCAG CGTCCTCACC GTCCTGCACC | |
| 1001 | AGGACTGGCT GAATGGCAAG GAGTACAAGT GCAAGGTCTC CAACAAAGCC | |
| 1051 | CTCCCAGCCC CCATCGAGAA AACCATCTCC AAAGCCAAAG GGCAGCCCCG | |
| 1101 | AGAACCACAG GTGTACACCC TGCCCCCATC CCGGGATGAG CTGACCAAGA | |
| 1151 | ACCAGGTCAG CCTGACCTGC CTGGTCAAAG GCTTCTATCC CAGCGACATC | |
| 1201 | GCCGTGGAGT GGGAGAGCAA TGGGCAGCCG GAGAACAACT ACAAGACCAC | |
| 1251 | GCCTCCCGTG TTGGACTCCG ACGGCTCCTT CTTCCTCTAC AGCAAGCTCA | |
| 1301 | CCGTGGACAA GAGCAGGTGG CAGCAGGGGA ACGTCTTCTC ATGCTCCGTG | |
| 1351 | ATGCATGAGG CTCTGCACAA CCACTACACG CAGAAGAGCC TCTCCCTGTC | |
| 1401 | TCCCGGTTGA | |

The deduced mature huP4A8-agly IgG1 heavy chain protein sequence encoded by pEAG2228 is shown below:

| | | |
|---|---|---|
| 1 | QVQLVQSGAE VKKPGASVKV SCKGSGYTFT DYGMHWVRQA PGQGLEWMGV | (SEQ ID NO: 48) |
| 51 | ISTYNGYTNY NQKFKGRVTM TVDKSTSTAY MELRSLRSDD TAVYYCARAY | |
| 101 | YGNLYYAMDY WGQGTLVTVS SASTKGPSVF PLAPSSKSTS GGTAALGCLV | |
| 151 | KDYFPEPVTV SWNSGALTSG VHTFPAVLQS SGLYSLSSVV TVPSSSLGTQ | |
| 201 | TYICNVNHKP SNTKVDKKVE PKSCDKTHTC PPCPAPELLG GPSVFLFPPK | |
| 251 | PKDTLMISRT PEVTCVVVDV SHEDPEVKFN WYVDGVEVHN AKTKPREEQY | |
| 301 | NSAYRVVSVL TVLHQDWLNG KEYKCKVSNK ALPAPIEKTI SKAKGQPREP | |
| 351 | QVYTLPPSRD ELTKNQVSLT CLVKGFYPSD IAVEWESNGQ PENNYKTTPP | |
| 401 | VLDSDGSFFL YSKLTVDKSR WQQGNVFSCS VMHEALHNHY TQKSLSLSPG | |

Characteristics of the humanized P4A8 IgG1 include: a solubility of over 12 mg/ml; pI (calculated) of 8.1; pI (IEF) of 9.1-9.2; the EC₅₀ of in vitro cytotoxicity of 30 ng/ml (WiDr cell MTT assay); the EC₅₀ for in vivo xenograft is 3.2 or 6.4 mg/kg depending on the animal model (as further shown herein); EC₅₀ of binding to WiDr cells by FACS is 0.12 nM.

### Example 14

### Binding Affinity

The EC₅₀ of hP4A8.IgG1 for Fn14 was estimated using an ELISA direct binding assay. 96 well ELISA plate was coated with 2 μg/ml of mouse Fn14-mouse Fc in sodium carbonate pH 9.5 overnight at 4° C. Plate was blocked with 3% BSA in PBS for 1 hour at room temperature. The concentrations of hP4A8.IgG1 were titrated from 2 μg/ml to 11 pg/ml and the incubation time was 1 hour at room temperature. The bound hP4A8.IgG1 was detected by HRP-goat anti-human IgG. The EC₅₀ for hP4A8.IgG1 under this ELISA condition is ˜6.79 ng/ml.

In another experiment, various isoforms of murine or humanized P4A8 were immobilized on CM5 sensorchips using the Biacore Amine Coupling kit according to manufacturer's instructions. Briefly, proteins were diluted to 30 μg/ml in 10 mM acetate, pH 5.0 and 10 μl was injected over chip surfaces that had been activated with a 10 μl injection of 1:1 N-hydroxsuccinimide (NHS): 1-Ethyl-3(3-dimethylaminopropyl)-carbodiimide hydrochloride (EDC). In addition one flow cell in each experiment was left underivitized as a background control. Excess free amine groups were then capped with a 50 μl injection of 1 M Ethanolamine. Typical immobilization levels were ˜1200 RU.

Concentration series ranging from 0.3 to 30 nM, of human Fn14 were prepared in Biacore buffer #1 (10 mM HEPES pH 7.0+150 mM NaCl+3.4 mM EDTA+0.005% P-20 detergent+0.05% BSA). The amino acid sequence of the soluble Fn14 protein used in these experiments was EQAPGTAPCSRGSSWSADLDKCMDCASCRARPHSD FCLGCAAAPPAPFRLLWPEQKLISEEDLHHHHHH. Samples were run over antibody and control surfaces in non-sequential order at a flow rate of 50 μl/min for 5 minutes followed by 15 minutes dissociation in Biacore buffer #1. After each cycle the chip was regenerated with 15 mM NaOH.

Raw data were normalized by setting the preinjection response to zero on the Y-axis and the injection start to zero on the X-axis for each concentration series. Data were further normalized by subtracting the response on the underivitized surface from the response on the active surfaces and then subtracting the buffer only response on the active surface from the binding data on the same surface (so-called ‘double referencing’ of the data). The global association and dissociation rate constants were then determined for each concentration series by fitting the data using a Marquardt-Levenberg algorithm for 1:1 binding within the Biaevaluation software. The affinity constant was calculated from the ratio of the rate constants (K_{D}=k_{d}/kₐ). The binding assays were done with greater than 95% pure monomeric soluble human Fn14.

Absorbance scan of human Fn14 was performed prior to binding assays. An extinction coefficient calculated from the amino acid sequence by the method of Pace et al. (Pace, C. N., Vajdos, F., Fee, L., Grimsley, G., and Gray, T. (1995) “How to measure and predict the molar absorption coefficient of a protein.” Protein Science, 4:2411-23.) was used.

Absorbance scans were performed prior to binding assays. Because the mAb is the immobilized species in these experiments accurate knowledge of the mass, concentration or molecular weight are not required for determination of accurate rate constants, therefore an approximate molecular weight of 150 kDa and an approximate mass extinction coefficient of 1.5 was used to estimate the concentrations of the antibodies from the absorbance at 280 nm.

TABLE 1
Affinity constant was calculated from the ratio of the rate constants (KD = kd/ka).

| P4A8 isoform | kₐ (M⁻¹ s⁻¹) | k_{d} (s⁻¹) | K_{D} (M) |
|---|---|---|---|
| Parental mAb (n = 6) | 8.2 ± 2.3 × 10⁵ | 1.6 ± 0.5 × 10⁻³ | 2.0 ± 0.6 × 10⁻⁹ |
| Murine IgG1 (n = 4) | 2.7 ± 1.1 × 10⁶ | 2.6 ± 0.3 × 10⁻³ | 1.1 ± 0.6 × 10⁻⁹ |
| Murine IgG2a (n = 6) | 2.4 ± 2.4 × 10⁶ | 3.4 ± 2.6 × 10⁻³ | 1.5 ± 1.5 × 10⁻⁹ |
| Chimeric (n = 2) | 5.5 ± 2.4 × 10⁵ | 1.5 ± 0.3 × 10⁻³ | 3.5 ± 2 × 10⁻⁹ |
| Murine IgG1 -agly (n = 2) | 5.6 ± 3 × 10⁵ | 1.5 ± 0.4 × 10⁻³ | 3.0 ± 1.3 × 10⁻⁹ |
| Humanized IgG1 (n = 5) | 1.7 ± 0.9 × 10⁶ | 2.9 ± 0.9 × 10⁻³ | 2.6 ± 2.1 × 10⁻⁹ |
| Humanized IgG1-RRS (n = 3) | 1.8 ± 0.3 × 10⁶ | 3 ± 0.1 × 10⁻³ | 1.7 ± 0.3 × 10⁻⁹ |
| Humanized IgG4(P) agly (n = 5) | 2.0 ± 1.6 × 10⁶ | 2.9 ± 0.7 × 10⁻³ | 3.4 ± 3.1 × 10⁻⁹ |
| Humanized IgG4(P) agly RRS (n = 3) | 2.9 ± 2 × 10⁶ | 4.7 ± 2 × 10⁻³ | 2.2 ± 1.3 × 10⁻⁹ |

The monovalent binding affinity (or “intrinsic affinity”) of humanized P4A8 to soluble monomeric human Fn14 is in the range of about 1 to 4 or 5 nM.

The bivalent binding affinity (affinity and avidity components) of P4A8 whole antibody to immobilized Fn14 (bivalent Fn14-Fc) is about 50 pM.

### Example 15

### Caspase Assay

The caspase assay measures levels of cleaved caspases 3 and 7. Induction of caspase cleavage was measured in response to treatment with hP4A8. Caspases 3 and 7 are considered to be the “executioner” caspases, immediately proximal to induction of apoptosis; and therefore, this assay is relevant to the proposed MOA of hP4A8.

WiDr tumor cells were seeded in 96-well plates, and exposed to a range of concentrations (1 μg/ml titrated at 1:3 dilutions) of hP4A8 in the presence of 80 U/ml of hIFNg. After 3 days in culture, the Promega Caspase-Glo 3/7 Assay reagent was used to measure the presence of cleaved caspases 3 and 7. The data are presented as fold change as compared to untreated cells.

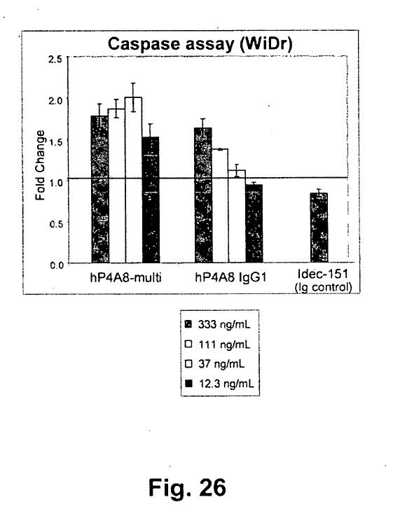

Results show induction of Caspases 3/7 in WiDr cells in response to stimulation with hP4A8, with a maximal effect observed in response to the multimeric version of hP4A8 (hP4A8-multi) even when tested at even the lowest concentration (FIG. 26). A dose response is observed when testing increasing concentrations of the monomeric form of P4A8. Similar results were obtained in ex vivo tumors.

### Example 16

### NFkB Induction Assay

The NF-kB assay measures induction of the canonical (p50, p65) and non-canonical (p52, RelB) NF-kB pathways. It has been well established that the TWEAK/Fn14 pathway signals through NF-kB; therefore, this is a relevant assay for demonstrating agonist activity of hP4A8.

WiDr tumor cells were grown in 6-well dishes and exposed to 1 μg/ml of P4A8 (in this assay the murine version of P4A8 was used), or 100 ng/ml hFc-TWEAK for comparison. At various time points post-treatment, ranging from 1 minute to 24 hours, nuclear extracts were prepared from the cultures. The nuclear extracts were then subjected to analysis by an ELISA kit (Active Motif—TransAM NFkB Family transcription factor Assay kit) to measure the individual NF-kB family members (p50, p65, p52, RelB, c-Rel). All values are normalized relative to unstimulated cells.

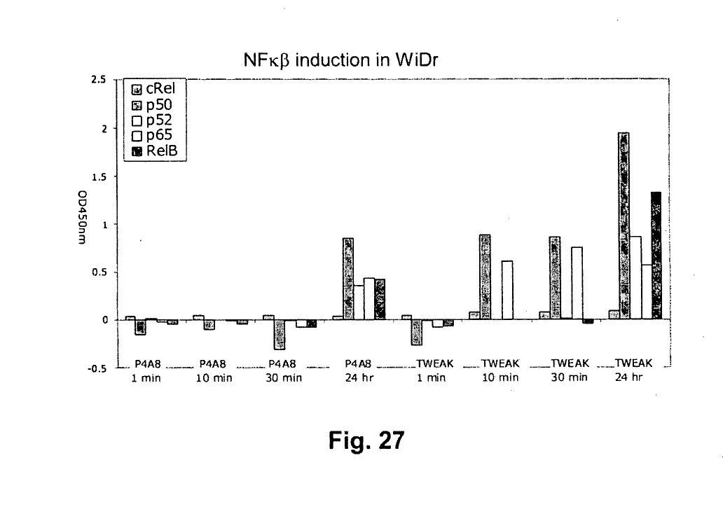

The results show induction of NFkB family members p50, p52, p65, and RelB in WiDr cells in response to P4A8, indicating stimulation of both the canonical and non-canonical NF-kB pathways (FIG. 27). Similar results were obtained in ex vivo tumors.

### Example 17

### Effector Function

ADCC activity of hP4A8 was assessed in vitro. Activity was measured in WiDr and MDA-MB231 tumor cell lines. hP4A8.IgG1 (i.e., humanized P4A8 having the VH1 and VL1 sequences linked to human IgG1), was compared to Fc-crippled versions of P4A8 (hP4A8-IgG1agly and hP4A8.IgG4Pagly).

NK cells isolated from donor PBMCs were incubated overnight in the presence of IL-2. WiDr and MDA-MB-231 target cells were labeled with ⁵¹Cr. Cultured NK cells and labeled target cells were incubated together at 5:1 ratio in the presence of varying concentrations of antibody for 4 hours at 37 degrees (also conducted at 2:1 ratio, data not shown). A spontaneous release control (no NK cells) and maximum release control (Triton-X-10 treated target cells) were included in the assay. Cpm in supernatant was measured following the incubation period. The % lysis was calculated as follows:

$$\% Lysis = \frac{( sample cpm - spont . cpm ) \times 100}{( max cpm - spont . cpm )}$$

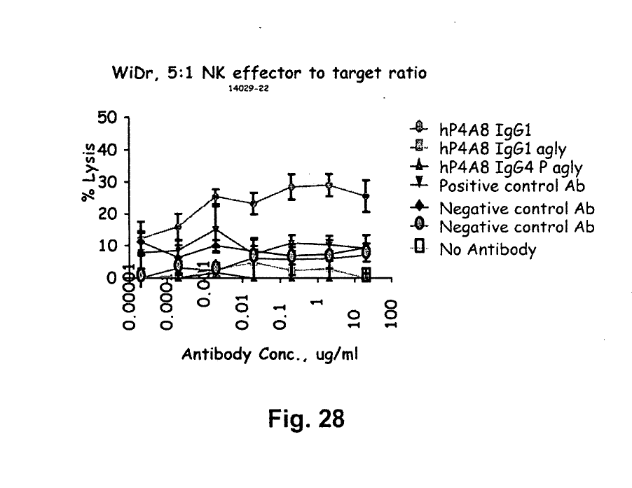

In both the WiDr and the MDA-MB231 experiments, significant ADCC activity was observed with the hIgG1 but not with the Fc crippled (hP4A8-IgG1agly and hP4A81gG4Pagly) P4A8 antibody. The positive controls showed some activity, though not as robust as hP4A8.IgG1 (FIG. 28). These studies demonstrate that hP4A8.IgG1 has ADCC capacity, as measured by the ability of the antibody to induce ADCC in the in vitro assay.

The effect of glycosylation on activity was also determined. The MTT assay (described above) in WiDr cells was used to test whether glycosylation has an effect on in vitro activity. hP4A8.IgG1 (full effector function) and hP4A8.IgG4 Pagly (no effector function) were compared in this assay. The Research Reference Standard materials were tested in this assay. Results show a slight but reproducible enhancement in activity of the hP4A8.IgG1 as compared to hP4A8.IgG4 Pagly in the in vitro assay.

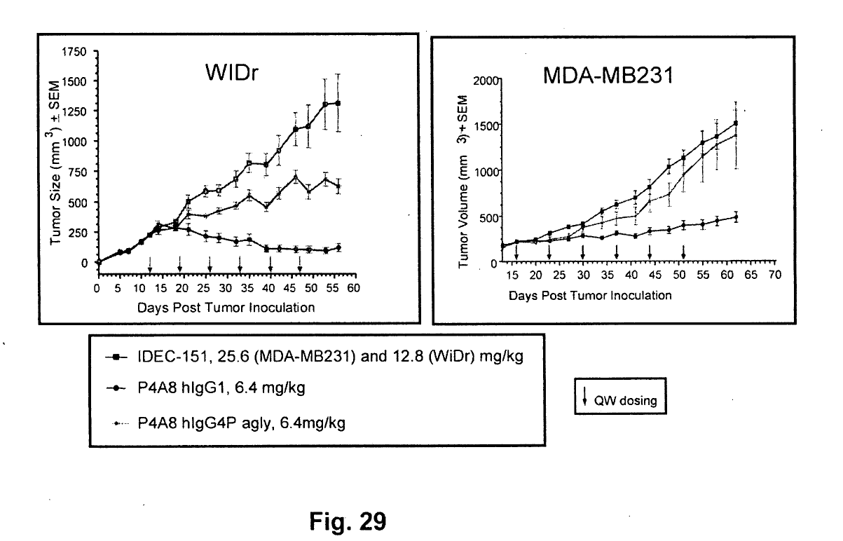

The Fc effector function of hP4A8.IgG1 has also been shown to contribute to P4A8 activity in vivo in both WiDr and MDA-MB231 xenograft assays. Administration of P4A8 hIgG1 at 6.4 mg/kg to either animal model is more efficacious than administration of P4A8hIgG4 Pagly at the same dose (FIG. 29).

### Example 18

### In Vivo Short and Long Term Efficacy of the Humanized P4A8.IgG1

Efficacy of P4A8.hIgG1 Fn14 antibody, administered as a single agent at doses ranging from 0.9 to 25.6 mg/kg administered intraperitoneally (i.p.) on a once a week schedule (qw) for 6 weeks was evaluated in WiDr human colon tumor-bearing athymic nude mice. Mice were treated with IDEC 151 (negative control) at 12.8 mg/kg and P4A8.hIgG1 at 12.8, 6.4, 3.2, 1.8 and 0.9 mg/kg IP, on a QW schedule (as indicated by arrows) starting on Day 12 following tumor cell inoculation when the average tumor volume was approximately 200 mm³. Data are Mean±SEM of 10 mice per treatment group. * p<0.001 compared to IDEC 151 negative control from Days 20 to 60 for all dosing groups.

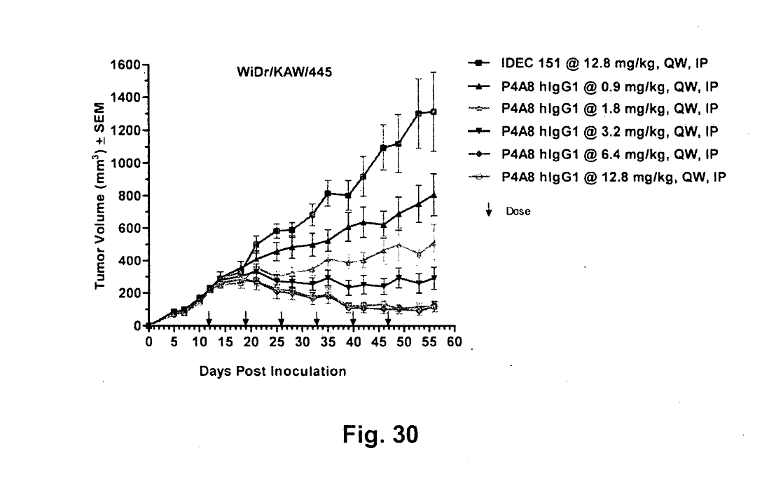

P4A8hIgG1 demonstrated statistically significant (p<0.001) efficacy at doses ranging from 0.9-25.6 mg/kg, compared to the isotype matched negative control antibody (FIG. 30, FIG. 31, and FIG. 32). Dose-dependent efficacy was observed across 0.9, 1.8, 3.2 and 6.4 mg/kg dose groups. Above 6.4 mg/kg dose, no dose-dependency was observed across 6.4, 12.8 and 25.6 mg/kg dose groups (FIG. 30 and FIG. 31). Across the dose range tested, the minimally efficacious dose of P4A8hIgG1, administered as a single agent in this model appears to be 0.9 mg/kg on a qwx6 schedule (FIG. 30 and FIG. 31). On the same dosing schedule the maximally efficacious dose is 6.4 mg/kg. As shown in FIG. 32, P4A8.hIgG1 antibody maintained efficacy for over 50 days following termination of dosing. All doses ranging from 0.9 to 25.6 mg/kg (n=10 mice/treatment group) were well tolerated on a qwx6 schedule as indicated by no body weight loss.

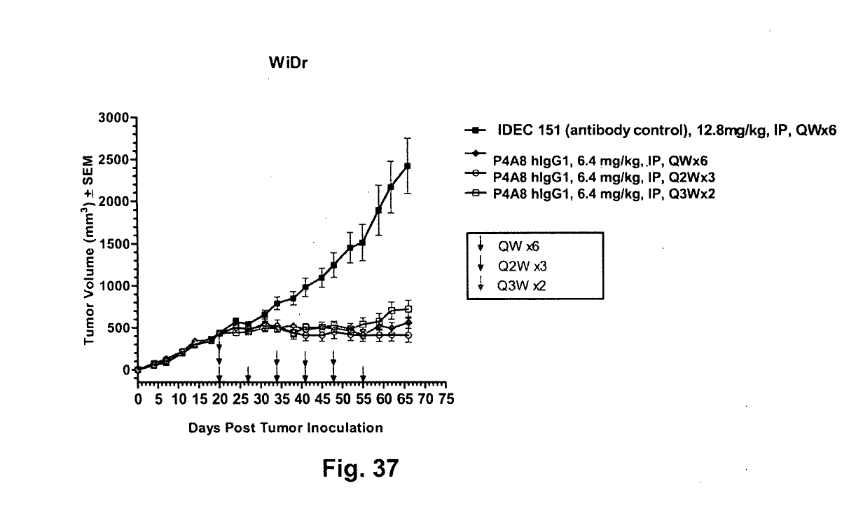

In addition to a weekly dosing schedule, administration of P4A8hIgG1 was also found to be effective in WiDr human colon tumor-bearing athymic nude mice when administered every other week or once every three weeks (FIG. 37). Treatment in this study began when the tumors were relatively large (approximately 500 mm³), and tumor stasis was still observed. Even though the half life of the antibody is within the normal to low range for antibodies (less than 2.5 days in tumor bearing mice), the antibody is surprisingly effective in vivo even if administered infrequently.

Efficacy of P4A8.hIgG1 Fn14 antibody, administered as a single agent at doses ranging from 6.4 to 25.6 mg/kg administered intraperitoneally (i.p) on a once a week schedule (qw) for 6 weeks was evaluated in the MDA-MB-231 breast carcinoma tumor-bearing SCID mice. MDA-MB-231 human breast tumor-bearing mice were treated with IDEC 151 (negative control) at 25.6 mg/kg and P4A8hIgG1 at 25.6, 12.8 and 6.4, mg/kg IP, on a QW schedule (as indicated by arrows) starting on Day 16 following tumor cell inoculation when the average tumor volume was approximately 200 mm³. Data are Mean±SEM of 9 mice per treatment group. * p<0.001 compared to IDEC 151 negative control from Days 23 to 63.

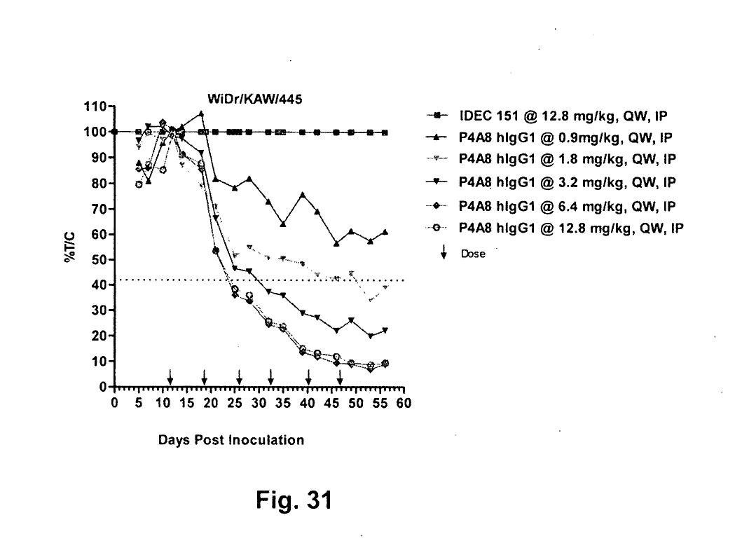

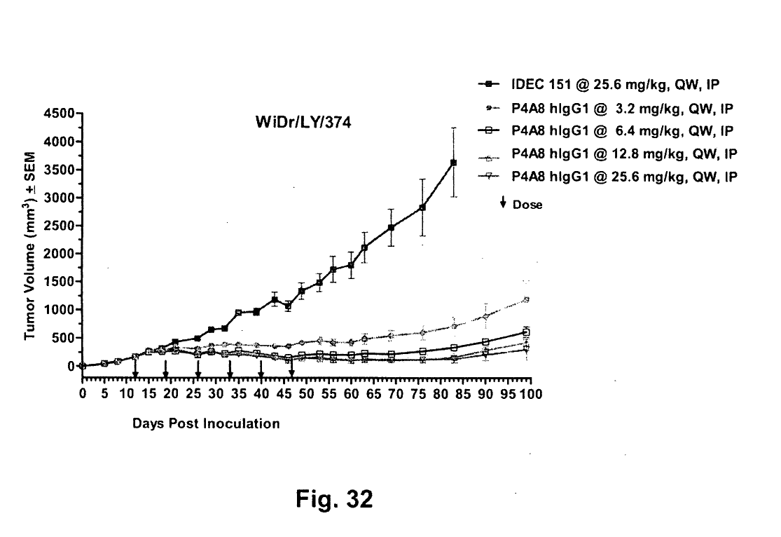

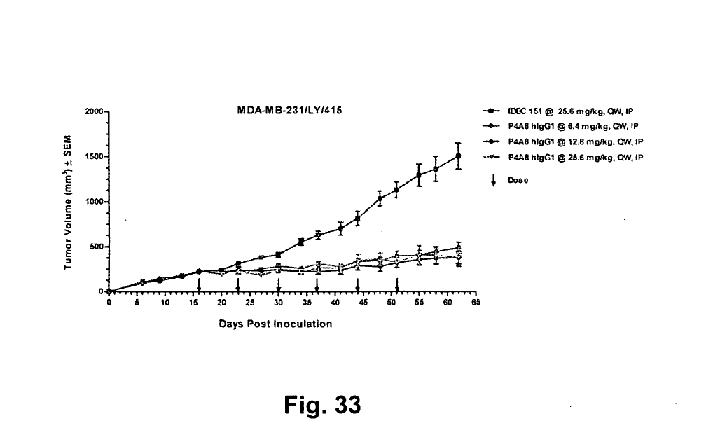

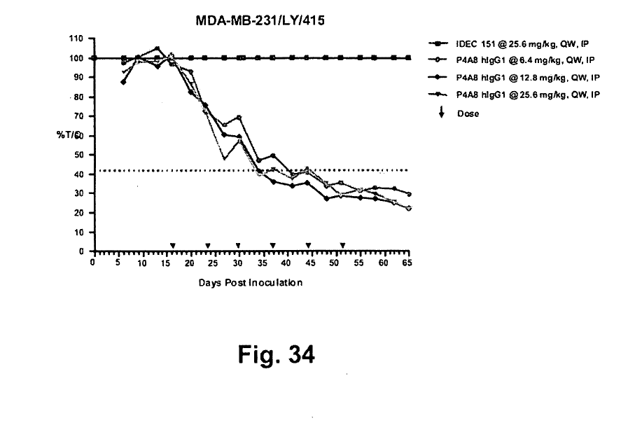

P4A8.hIgG1 demonstrated statistically significant (p<0.001) efficacy at doses ranging from 6.4-25.6 mg/kg, compared to the isotype matched negative control antibody (FIG. 33). Comparison of the test group mean tumor sizes as a percentage of the mean negative control are presented in FIG. 34, the dotted line indicates the National Cancer Institute's criteria for activity (42%).

The minimally efficacious dose of P4A8.hIgG1, when administered as a single agent, has not yet been determined for this model. No dose-dependency was observed across 6.4, 12.8 and 25.6 mg/kg dose groups. These doses were well tolerated as indicated by no significant body weight loss.

Unexpectedly, P4A8.hIgG1 exhibited greater efficacy in the MDA-MB-231 human breast tumor assay than did the parent antibody P4A8. The two antibodies exhibited similar efficacy in the WiDr human colon tumor assay.

### Example 19

### Multimerization of P4A8.hIgG1 Enhances Activity

Multimerization of P4A8.hIgG1 with Protein A enhanced WiDr cell death in an MTT assay, as well as Caspase activation in WiDr cells (FIG. 15 and FIG. 26).

### Example 20

### Efficacy of Humanized P4A8 IgG1 in Gastric Carcinoma

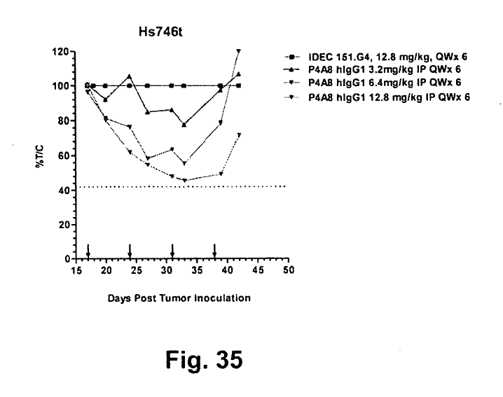

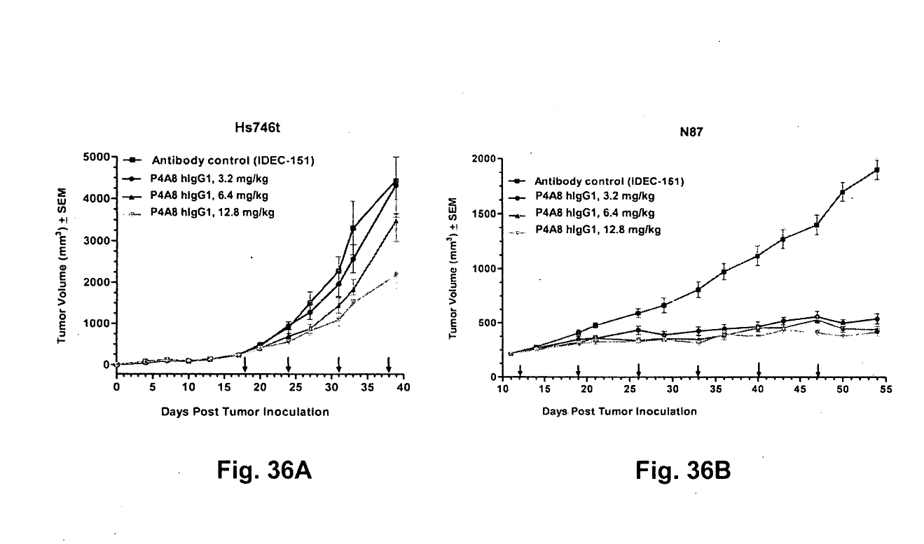

The humanized P4A8 IgG1 antibody was shown to exhibit an anti-tumor effect at various doses tested in the Hs746T gastric carcinoma xenograft model (FIG. 35 and FIG. 36A). In addition, single agent efficacy (70-80% reduction in tumor size) was demonstrated by treatment with humanized P4A8IgG1 at 3.2, 6.4 and 12.8 mg/kg with once weekly dosing in the N87 gastric xenograft model (FIG. 36B).

Thus, P4A8 effectively kills tumor cells in in vivo animal models, and has a prolonged effect.

### Example 21

### Amino Acid Residues at the Interface of the P4A8 Fn14 Interaction

The complex of the murine P4A8 Fab/human Fn14 ectodomain was crystallized by vapor diffusion method and placed at a temperature of 20° C. Plate-shaped crystals of diffraction quality grew in 10-14 days in a crystallization solution that contained 30% PEG 8000, 100 mM sodium acetate at pH 5, 0.2 M lithium sulfate. Crystals (0.2×0.2×0.01 mm³) were harvested as is and flash frozen in liquid nitrogen. Diffraction data to 3.5 Å resolution was collected at beamline X25 at the National Synchrotron Light Source (Upton, N.Y.). Data processing with the HKL2000 program (HKL Research, Charlottesville, Va., USA) revealed the crystals to belong to a P21 space group and approximate cell dimensions a=61.1 Å, b=103.3 Å, c=76.1 Å, and β=97.2°, consistent with 2 P4A8 Fab-Fn14 complexes per asymmetric unit. Molecular replacement with MOLREP (Vagin & Teplyakov, J Appl Crystallogr 1997; 30:1022-1025) utilizing a homology model of the humanized P4A8 and an in-house Fn14 NMR structure led to placement of the P4A8 Fab and the Fn14 molecules with a resulting R-factor of 46%. Only residues 50-67 of Fn14's cysteine rich domain could be accounted for in the electron density maps. Missing from the density were H₃CDR and the N-terminal residues of Fn14 ectodomain. A more complete model of the interface was generated by superposing the recent NMR structure of huFn14 ectodomain (He & Dang, Protein Science 2009; 18:650-656) to that of the P4A8 Fab/Fn14 crystal structure. This was followed by modeling of the H3CDR with software ROSETTA (Das & Baker, Annu. Rev. Biochem., 2008; 77:363-82) and a constrained optimization refinement of the overall complex. Table 2 highlights the amino acid interactions at the P4A8/Fn14 interface.

TABLE 2
Amino Acid Interactions at the P4A8/Fn14 Interface

| CDR L1 | CDR L2 | CDR L3 |
|---|---|---|
| RASKSVSTSSYSYMH | YASNLES | SRELPFT |
| S32 (P4A8) | Y54 (P4A8) | R96 (P4A8) |
| *C49 (Fn14) | *K48 (Fn14) | D51 (Fn14) |
| Y34 (P4A8) | | |
| W42 (Fn14) | | |
| Y36 (P4A8) | | |
| K48 (Fn14) | | |

| CDR H1 | CDR H2 | CDR H3 |
|---|---|---|
| GYTFTDYGMH | VISTYNGYTNYNQKFKG | AYYGNLYYAMDY |
| D31 (P4A8) | S52 (P4A8) | Y101 (P4A8) |
| *R58 (Fn14) | *A57 (Fn14) | L46 (Fn14) |
| Y32 (P4A8) | Y54 (P4A8) | Y105 (P4A8) |
| R58 (Fn14) | H60 (Fn14) | M50 (Fn14) |
| | N55 (P4A8) | Y106 (P4A8) |
| | *A57 (Fn14) | R58 (Fn14) |
| | Y57 (P4A8) | |
| | R56 (Fn14) | |
| | N59 (P4A8) | |
| | *R56 (Fn14) | |

CDRs of P4A8 with interface residues highlighted in bold/underlined.
*indicates H-bond interaction

### Example 22

### Sensitivity of Cell Lines to P4A8, P4A8 Multimer, and TWEAK

FACS analysis of cell lines was done in FACS buffer (PBS 1% BSA 0.1% Na Azide) by mixing cells with a dose curve of P4A8, starting at 10 μg/ml followed by a serial dilution of 1:2. As a control mAb IDEC 151 was prepared in the same manner and then each antibody was incubated with the cells for 30 min at 4° C. Following 2 washes with FACS buffer the cells were incubated with PE labeled anti hu IgG Fc specific antibody (Jackson Labs West Grove, Pa.) 30 min 4 C. Following 2 washes the cells were fixed in 2% para formaldehyde and acquired on Caliber Facscan (Becton Dickinson, San Jose, Calif.). The data was analyzed using Flow Jo software (Tree Star Inc. Ashland, Oreg.) and the MFI's (Mean Fluorescent Intensity) were determined. The expression levels of the cell lines (see Table 3) were scored according to their MFI at a concentration of 1.25 μg/ml P4A8 by the following criteria:

| | | |
|---|---|---|
| Negative | <10 | MFI |
| Low | 10-29 | MFI |
| Medium | 30-59 | MFI |
| High | 60+ | MFI |

The MTT assays were set up by plating the cells in media containing 80 U/ml human INFg along with a 1:3 serial dilution of Fc-Tweak, hP4A8 IgG1, hP4A8 IgG1 multimer, or IDEC 151 control mAb starting at 9 μg/ml in triplicate. The cells were incubated for 3-4 days and developed using One Solution Cell Titer MTT assay (Promega Madison Wis.). The percentage survival was determined by using the formula: % Survival=(OD of treated wells/average OD of the untreated wells)*100, for each individual sample. An average was calculated for each treatment condition and the % survival was then plotted vs. concentration of inhibitor.

The results of the MTT assays (see Table 3) were scored by their ability to inhibit proliferation at 9 μg/ml using the following criteria:

No Activity − (negative)

>80% Survival +/−

˜10-80% Survival +

˜60% Survival ++

˜40% Survival +++

˜<20% Survival ++++

TABLE 3
Cell Line Sensitivity P4A8, P4A8 Multimer, and TWEAK

| | | | MTT sensitivity | | |
|---|---|---|---|---|---|
| Tumor | | Expression | | P4A8- | |
| type | Cell line | (FACS) | P4A8 | multimer | TWEAK |
| colon | WiDr | medium | +++ | ++++ | ++++ |
| | HT-29 | medium | +++ | ND | ++++ |
| | HCT-15 | medium | +/− | ND | +/− |
| | HCT-116 | medium | + | + | + |
| | SW-620 | medium | − | +/− | + |
| | Geo | medium | − | + | ++ |
| | Dld-1 | medium | − | − | − |
| | Lovo | low | − | ND | ++ |
| | Km-12 | low | − | − | + |
| | Colo-205 | negative | − | − | − |
| breast | NCI-ADR | very high | +/− | +/− | + |
| | MDA- | medium | +/− | + | ++ |
| | MB231 | | | | |
| | SUM-159 | medium | − | ND | − |
| | Mx1 | medium | + | ++ | ++ |
| | DU4475 | low | − | ND | − |
| | BT-549 | negative | − | ND | − |
| | ZR-75-1 | negative | − | − | − |
| | MCF-7 | ND | +/− | ND | ++ |
| pancreas | BxPc-3 | medium | +/− | + | +++ |
| | CFPAC-1 | medium | − | ND | − |
| | Su86.86 | medium | − | − | + |
| | Panc-1 | low/med | +/− | +/− | + |
| | SW1990 | medium | − | +/− | +/− |
| | AsPC-1 | low | + | + | + |
| | HCC1806 | high | +/− | + | + |
| gastric | Hs746T | medium | − | ++ | ++ |
| | NCI-N87 | medium | − | ++ | ++ |
| ovarian | ES-2 | high | +/− | +/− | +/− |
| | SKOV-3 | medium | + | ++ | +++ |
| NSCL | HOP62 | medium/high | +/− | + | ++ |
| | A549 | medium | +/− | +/− | +/− |
| | NCI-H23 | low | +/− | + | + |
| melanoma | MDA- | medium | − | ND | − |
| | MB435 | | | | |
| | SK-MEL-2 | medium | +/− | ++ | ++ |

ND = not done

### Example 23

### Antibody Crossblocking

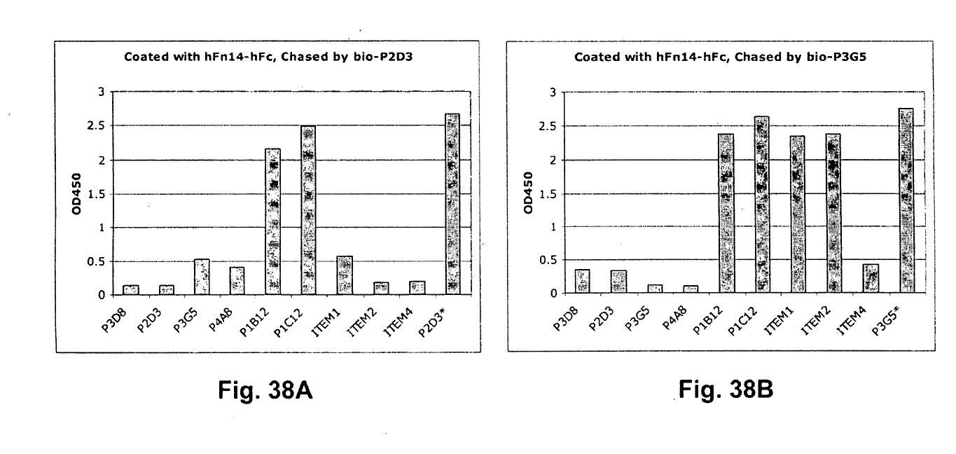

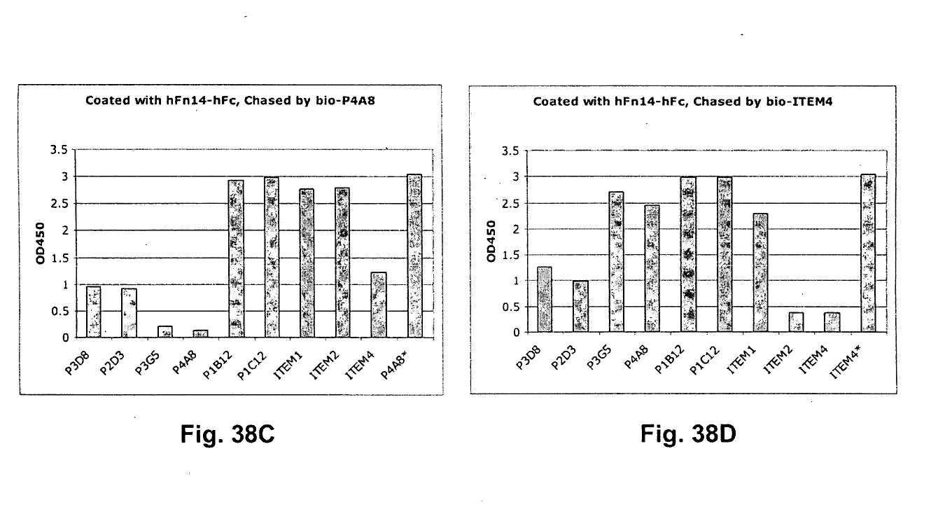

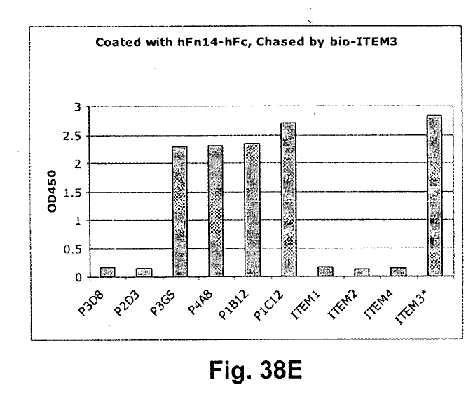

Antibody crossblocking was evaluated as follows. Soluble human Fn14 was immobilized on a surface. The surface was then contacted with an unlabeled first antibody. Subsequently, a biotinylated second antibody was added and binding of the second antibody to the surface was measured. An abrogation of second antibody binding indicated that the first antibody crossblocked binding of the second antibody to Fn14. The ability of a panel of antibodies to crossblock binding of selected anti-Fn14 antibodies is depicted in FIG. 38A (P2D3 was the biotinylated second antibody), FIG. 38B (P3G5 was the biotinylated second antibody), FIG. 38C (P4A8 was the biotinylated second antibody), FIG. 38D (ITEM-4 was the biotinylated second antibody), and FIG. 38E (ITEM-3 was the biotinylated second antibody). In these experiments, P1B12 and P1C12 were used as unrelated control antibodies. * indicates instances were no unlabeled first antibody was used.

The following is a summary of the protocol used in these crossblocking experiments.

1. Coat plate with 0.1 ug/ml hFn14-hFc in 0.1 M carbonate, pH9.5, using Corning Costar 3590 overnight at 4° C.

2. Block with 3% BSA in PBS (200 ul/well) for 1 hour at RM.

3. Wash 3× with wash buffer (0.1% Tween-20 in PBS).

4. Add anti-Fn14 antibody at 10 ug/ml horizontally in cross 96 well plate (1-12), 100 ul per well and incubate for 1 hour.

5. Without wash, add anti-Fn14 antibody biotinylated at 0.2 ug/ml vertically cross 96 well plate (A-H), 100 ul per well and incubate for 1 hour.

6. Wash 3× with wash buffer (0.1% Tween-20 in PBS).

7. Add HRP-SA at 1:2000, apply 100 ul per well, incubate at RM for 1 hour.

8. Prepare TMB Substrate Solution by mixing 1 to 1 ratio of reagent A and reagent B (TMB Substrate Reagent Set, BD Biosciences 555214). Add 100 ul per well.

9. Read at 405 nm when color developed.

10. Stop the reaction with 100 ul 2N H2SO4 and read at 450 mm.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated antibody or antigen-binding fragment thereof that (i) selectively binds to the polypeptide of SEQ ID NO:1, when expressed on the surface of a cell, at an epitope that includes the amino acid residue tryptophan at position 42 of SEQ ID NO:1, and (ii) induces or enhances cell killing of cancer cells in vivo or in vitro.

2. An isolated antibody or antigen-binding fragment thereof that (i) selectively binds to the polypeptide of SEQ ID NO:1, when expressed on the surface of a cell, and crossblocks binding of the monoclonal antibody P4A8 or P3G5 to SEQ ID NO:1, and (ii) induces or enhances cell killing of cancer cells in vivo or in vitro.

3. An isolated antibody or antigen-binding fragment thereof that (i) selectively binds to the polypeptide of SEQ ID NO:1, when expressed on the surface of a cell, at the same epitope as the monoclonal antibody P4A8, P3G5, or P2D3, and (ii) induces or enhances cell killing of cancer cells in vivo or in vitro.

4. The antibody or antigen-binding fragment thereof of claim 1, wherein binding of the antibody or antigen-binding fragment thereof to the polypeptide of SEQ ID NO:1 blocks binding of TWEAK to the polypeptide.

5. An isolated antibody or antigen-binding fragment thereof that (i) selectively binds to the polypeptide of SEQ ID NO:1, when expressed on the surface of a cell, (ii) comprises a VH domain that is at least 80% identical to the amino acid sequence of SEQ ID NO:11 or SEQ ID NO:12, and (iii) induces or enhances cell killing of cancer cells in vivo or in vitro.

6. The antibody or antigen-binding fragment thereof of claim 5, wherein the VH domain is at least 90% identical to the amino acid sequence of SEQ ID NO:11 or SEQ ID NO:12.

7. The antibody or antigen-binding fragment thereof of claim 6, wherein the VH domain is at least 95% identical to the amino acid sequence of SEQ ID NO:11 or SEQ ID NO:12.

8. The antibody or antigen-binding fragment thereof of claim 5, wherein the VH domain is identical to the amino acid sequence of SEQ ID NO:11 or SEQ ID NO:12.

9. An isolated antibody or antigen-binding fragment thereof that (i) selectively binds to the polypeptide of SEQ ID NO:1, when expressed on the surface of a cell, (ii) comprises a VL domain that is at least 80% identical to the amino acid sequence of SEQ ID NO:13, SEQ ID NO:14, or SEQ ID NO:15, and (iii) induces or enhances cell killing of cancer cells in vivo or in vitro.

10. The antibody or antigen-binding fragment thereof of claim 9, wherein the VL domain is at least 90% identical to the amino acid sequence of SEQ ID NO:13, SEQ ID NO:14, or SEQ ID NO:15.

11. The antibody or antigen-binding fragment thereof of claim 9, wherein the VL domain is at least 95% identical to the amino acid sequence of SEQ ID NO:13, SEQ ID NO:14, or SEQ ID NO:15.

12. The antibody or antigen-binding fragment thereof of claim 9, wherein the VL domain is identical to the amino acid sequence of SEQ ID NO:13, SEQ ID NO:14, or SEQ ID NO:15.

13. An isolated antibody or antigen-binding fragment thereof that (i) selectively binds to the polypeptide of SEQ ID NO:1, when expressed on the surface of a cell, (ii) comprises a VH domain that is at least 80% identical to the amino acid sequence of SEQ ID NO:11 or SEQ ID NO:12, (iii) comprises a VL domain that is at least 80% identical to the amino acid sequence of SEQ ID NO:13, SEQ ID NO:14, or SEQ ID NO:15, and (iv) induces or enhances cell killing of cancer cells in vivo or in vitro.

14. The antibody or antigen-binding fragment thereof of claim 13, wherein (i) the VH domain is at least 90% identical to the amino acid sequence of SEQ ID NO:11 or SEQ ID NO:12, and (ii) the VL domain is at least 90% identical to the amino acid sequence of SEQ ID NO:13, SEQ ID NO:14, or SEQ ID NO:15.

15. The antibody or antigen-binding fragment thereof of claim 13, wherein (i) the VH domain is at least 95% identical to the amino acid sequence of SEQ ID NO:1 or SEQ ID NO:12, and (ii) the VL domain is at least 95% identical to the amino acid sequence of SEQ ID NO:13, SEQ ID NO:14, or SEQ ID NO:15.

16. The antibody or antigen-binding fragment thereof of claim 13, wherein (i) the VH domain is identical to the amino acid sequence of SEQ ID NO:11 or SEQ ID NO:12, and (ii) the VL domain is identical to the amino acid sequence of SEQ ID NO:13, SEQ ID NO:14, or SEQ ID NO:15.

17. The antibody or antigen-binding fragment thereof of claim 13, wherein the heavy chain comprises SEQ ID NO:37 or SEQ ID NO:39 and the light chain comprises SEQ ID NO:41, SEQ ID NO:43, or SEQ ID NO:45.

18. The antibody or antigen-binding fragment thereof of claim 13, wherein the heavy chain comprises SEQ ID NO:37 and the light chain comprises SEQ ID NO:43.

19. An isolated antibody or antigen-binding fragment thereof that (i) selectively binds to the polypeptide of SEQ ID NO:1, when expressed on the surface of a cell, (ii) comprises a VH domain comprising (a) a first heavy chain complementarity determining region (CDR) that is at least 90% identical to CDR-H1 of SEQ ID NO:2 or SEQ ID NO:3, a second heavy chain CDR that is at least 90% identical to CDR-H2 of SEQ ID NO:2 or SEQ ID NO:3, and a third heavy chain CDR that is at least 90% identical to CDR-H3 of SEQ ID NO:2 or SEQ ID NO:3, or (b) a first heavy chain CDR that is at least 90% identical to CDR-H1 of SEQ ID NO:4, a second heavy chain CDR that is at least 90% identical to CDR-H2 of SEQ ID NO:4, and a third heavy chain CDR that is at least 90% identical to CDR-H3 of SEQ ID NO:4, and (iii) induces or enhances cell killing of cancer cells in vivo or in vitro.

20. The antibody or antigen-binding fragment thereof of claim 19, wherein the first heavy chain CDR is identical to CDR-H1 of SEQ ID NO:2 or SEQ ID NO:3, the second heavy chain CDR is identical to CDR-H2 of SEQ ID NO:2 or SEQ ID NO:3, and the third heavy chain CDR is identical to CDR-H3 of SEQ ID NO:2 or SEQ ID NO:3.

21. The antibody or antigen-binding fragment thereof of claim 19, wherein the first heavy chain CDR is identical to CDR-H1 of SEQ ID NO:4, the second heavy chain CDR is identical to CDR-H2 of SEQ ID NO:4, and the third heavy chain CDR is identical to CDR-H3 of SEQ ID NO:4.

22. An isolated antibody or antigen-binding fragment thereof that (i) selectively binds to the polypeptide of SEQ ID NO:1, when expressed on the surface of a cell, (ii) comprises a VL domain comprising (a) a first light chain CDR that is at least 90% identical to CDR-L1 of SEQ ID NO:5 or SEQ ID NO:6, a second light chain CDR that is at least 90% identical to CDR-L2 of SEQ ID NO:5 or SEQ ID NO:6, and a third light chain CDR that is at least 90% identical to CDR-L3 of SEQ ID NO:5 or SEQ ID NO:6, or (b) a first light chain CDR that is at least 90% identical to CDR-L1 of SEQ ID NO:7, a second light chain CDR that is at least 90% identical to CDR-L2 of SEQ ID NO:7, and a third light chain CDR that is at least 90% identical to CDR-L3 of SEQ ID NO:7, and (iii) induces or enhances cell killing of cancer cells in vivo or in vitro.

23. The antibody or antigen-binding fragment thereof of claim 22, wherein the first light chain CDR is identical to CDR-L1 of SEQ ID NO:5 or SEQ ID NO:6, the second light chain CDR is identical to CDR-L2 of SEQ ID NO:5 or SEQ ID NO:6, and the third light chain CDR is identical to CDR-L3 of SEQ ID NO:5 or SEQ ID NO:6.

24. The antibody or antigen-binding fragment thereof of claim 22, wherein the first light chain CDR is identical to CDR-L1 of SEQ ID NO:7, the second light chain CDR is identical to CDR-L2 of SEQ ID NO:7, and the third light chain CDR is identical to CDR-L3 of SEQ ID NO:7.

25. An isolated antibody or antigen-binding fragment thereof that (i) selectively binds to the polypeptide of SEQ ID NO:1, when expressed on the surface of a cell, (ii) comprises a VH domain comprising (a) a first heavy chain CDR that is at least 90% identical to CDR-H1 of SEQ ID NO:2 or SEQ ID NO:3, a second heavy chain CDR that is at least 90% identical to CDR-H2 of SEQ ID NO:2 or SEQ ID NO:3, and a third heavy chain CDR that is at least 90% identical to CDR-H3 of SEQ ID NO:2 or SEQ ID NO:3, or (b) a first heavy chain CDR that is at least 90% identical to CDR-H1 of SEQ ID NO:4, a second heavy chain CDR that is at least 90% identical to CDR-H2 of SEQ ID NO:4, and a third heavy chain CDR that is at least 90% identical to CDR-H3 of SEQ ID NO:4, (iii) comprises a VL domain comprising (a) a first light chain CDR that is at least 90% identical to CDR-L1 of SEQ ID NO:5 or SEQ ID NO:6, a second light chain CDR that is at least 90% identical to CDR-L2 of SEQ ID NO:5 or SEQ ID NO:6, and a third light chain CDR that is at least 90% identical to CDR-L3 of SEQ ID NO:5 or SEQ ID NO:6, or (b) a first light chain CDR that is at least 90% identical to CDR-L1 of SEQ ID NO:7, a second light chain CDR that is at least 90% identical to CDR-L2 of SEQ ID NO:7, and a third light chain CDR that is at least 90% identical to CDR-L3 of SEQ ID NO:7, and (iv) induces or enhances cell killing of cancer cells in vivo or in vitro.

26. The antibody or antigen-binding fragment thereof of claim 25, wherein (i) the first heavy chain CDR is identical to CDR-H1 of SEQ ID NO:2, the second heavy chain CDR is identical to CDR-H2 of SEQ ID NO:2, and the third heavy chain CDR is identical to CDR-H3 of SEQ ID NO:2, and (ii) the first light chain CDR is identical to CDR-L1 of SEQ ID NO:5, the second light chain CDR is identical to CDR-L2 of SEQ ID NO:5, and the third light chain CDR is identical to CDR-L3 of SEQ ID NO:5.

27. The antibody or antigen-binding fragment thereof of claim 25, wherein (i) the first heavy chain CDR is identical to CDR-H1 of SEQ ID NO:3, the second heavy chain CDR is identical to CDR-H2 of SEQ ID NO:3, and the third heavy chain CDR is identical to CDR-H3 of SEQ ID NO:3, and (ii) the first light chain CDR is identical to CDR-L1 of SEQ ID NO:6, the second light chain CDR is identical to CDR-L2 of SEQ ID NO:6, and the third light chain CDR is identical to CDR-L3 of SEQ ID NO:6.

28. The antibody or antigen-binding fragment thereof of claim 25, wherein (i) the first heavy chain CDR is identical to CDR-H1 of SEQ ID NO:4, the second heavy chain CDR is identical to CDR-H2 of SEQ ID NO:4, and the third heavy chain CDR is identical to CDR-H3 of SEQ ID NO:4, and (ii) the first light chain CDR is identical to CDR-L1 of SEQ ID NO:7, the second light chain CDR is identical to CDR-L2 of SEQ ID NO:7, and the third light chain CDR is identical to CDR-L3 of SEQ ID NO:7.

29. The antibody or antigen-binding fragment thereof of claim 25, wherein the antibody or antigen-binding fragment thereof comprises framework regions that are collectively at least 90% identical to human germline framework regions.

30. The antibody or antigen-binding fragment thereof of claim 25, wherein the antibody or antigen-binding fragment thereof comprises VH domain framework regions that are collectively at least 90% identical to the framework regions of the VH domain of SEQ ID NO:11 or SEQ ID NO:12.

31. The antibody or antigen-binding fragment thereof of claim 25, wherein the antibody or antigen-binding fragment thereof comprises VL domain framework regions that are collectively at least 90% identical to the framework regions of the VL domain of SEQ ID NO:13, SEQ ID NO:14, or SEQ ID NO:15.

32. The antibody or antigen-binding fragment thereof of claim 25, wherein the antibody or antigen-binding fragment thereof comprises (i) VH domain framework regions that are collectively at least 90% identical to the framework regions of the VH domain of SEQ ID NO:11 or SEQ ID NO:12, and (ii) VL domain framework regions that are collectively at least 90% identical to the framework regions of the VL domain of SEQ ID NO:13, SEQ ID NO:14, or SEQ ID NO:15.

33. The antibody or antigen-binding fragment thereof of claim 25, wherein the VH domain comprises amino acids 1-121 of SEQ ID NO:8.

34. The antibody or antigen-binding fragment thereof of claim 25, wherein the VL domain comprises amino acids 1-111 of SEQ ID NO:9.

35. The antibody or antigen-binding fragment thereof of claim 25, wherein the VH domain comprises amino acids 1-121 of SEQ ID NO:8 and the VL domain comprises amino acids 1-111 of SEQ ID NO:9.

36. The antibody or antigen-binding fragment thereof of claim 25, wherein the heavy chain comprises SEQ ID NO:8 and the light chain comprises SEQ ID NO:9.

37. The antibody or antigen-binding fragment thereof of claim 25, wherein the heavy chain comprises SEQ ID NO:16 and the light chain comprises SEQ ID NO:9.

38. The antibody or antigen-binding fragment thereof of claim 1, wherein the antibody or antigen-binding fragment thereof induces or enhances cell killing of WiDr colon cancer cells.

39. The antibody or antigen-binding fragment thereof of claim 1, wherein the antibody is a humanized antibody.

40. The antibody or antigen-binding fragment thereof of claim 1, wherein the antibody is a fully human antibody.

41. The antibody or antigen-binding fragment thereof of claim 1, wherein the antibody is a monoclonal antibody.

42. The antibody or antigen-binding fragment thereof of claim 1, wherein the antibody is a single chain antibody.

43. The antibody or antigen-binding fragment thereof of claim 1, wherein the antibody or antigen-binding fragment thereof is a polyclonal antibody, a chimeric antibody, an F_{ab} fragment, an F_{(ab′)2} fragment, an F_{ab′} fragment, an F_{sc} fragment, or an Fᵥ fragment.

44. The antibody or antigen-binding fragment thereof of claim 1, wherein the antibody or antigen-binding fragment thereof is a multispecific antibody.

45. The antibody or antigen-binding fragment thereof of claim 44, wherein the multispecific antibody is a bispecific antibody.

46. The antibody or antigen-binding fragment thereof of claim 1, wherein the antibody or antigen-binding fragment thereof is a multivalent antibody.

47. The antibody or antigen-binding fragment thereof of claim 1, wherein the antibody has an IgG1 heavy chain constant region.

48. An isolated cell that produces the antibody or antigen-binding fragment thereof of claim 1.

49. The cell of claim 48, wherein the cell is a fused cell obtained by fusing a mammalian B cell and myeloma cell.

50. A pharmaceutical composition comprising the antibody or antigen-binding fragment thereof of claim 1 and a pharmaceutically acceptable carrier.

51. A method of inducing death of a tumor cell, the method comprising contacting a tumor cell that expresses Fn14 with an amount of the antibody or antigen-binding fragment thereof of claim 1 effective to induce death of the tumor cell.

52. A method of preventing or reducing tumor cell growth, the method comprising administering to a mammal having a tumor a pharmaceutical composition comprising an amount of the antibody or antigen-binding fragment thereof of claim 1 effective to prevent or reduce tumor cell growth.

53. A method of treating a cancer, the method comprising administering to a mammal having a cancer a pharmaceutical composition comprising a therapeutically effective amount of the antibody or antigen-binding fragment thereof of claim 1.

54. The method of claim 53, wherein the cancer is a colon cancer or a breast cancer.

55. The method of claim 52, wherein the mammal is a human.


SEQUENCE LISTING (59 sequences)

SEQ ID NO: 1 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 2 - protein, 121 aa, Artificial Sequence
  Description of Artificial Sequence Synthetic polypeptide
QVQLQQSGPEVVRPGVSVKISCKGSGYTFTDYGMHWVKQSHAKSLEWIGVISTYNGYTNYNQKFKGKATMTVDKSSSTAYMELARLTSEDSAIYYCARAYYGNLYYAMDYWGQGTSVTVSS

SEQ ID NO: 3 - protein, 121 aa, Artificial Sequence
  Description of Artificial Sequence Synthetic polypeptide
QVQLQQSGPEVVRPGVSVKISCKGSGYTFTDYGIHWVKQSHAKSLEWIGVISTYNGYTNYNQKFKGKATMTVDKSSSTAYMELARLTSEDSAIYYCARAYYGNLYYAMDYWGQGTSVTVSS

SEQ ID NO: 4 - protein, 123 aa, Artificial Sequence
  Description of Artificial Sequence Synthetic polypeptide
QVSLKESGPGILQPSQTLSLTCSFSGFSLSTSGMGVSWIRQPSGKGLEWLAHIYWDDDKRYNPSLKSRLTISKDTSRNQVFLKITSVDTADTATYYCARRGPDYYGYYPMDYWGQGTSVTVSS

SEQ ID NO: 5 - protein, 111 aa, Artificial Sequence
  Description of Artificial Sequence Synthetic polypeptide
DIVLTQSPASLAVSLGQRATISCRASKSVSTSSYSYMHWYQQKPGQPPKLLIKYASNLESGVPARFSGSGSGTDFILNIHPVEEEDAATYYCQHSRELPFTFGSGTKLEIK

SEQ ID NO: 6 - protein, 111 aa, Artificial Sequence
  Description of Artificial Sequence Synthetic polypeptide
DIVLTQSPASLAVSLGQRATISCRANKSVSTSSYSYMHWYQQKPGQPPKLLIKYASNLESGVPARFSGSGSGTDFILNIHPVEEEDAATYYCQHSRELPFTFGSGTKLEIK

SEQ ID NO: 7 - protein, 111 aa, Artificial Sequence
  Description of Artificial Sequence Synthetic polypeptide
DIVLTQSPASLAVSLGQRATISCRASKSVSTSSYSYMHWYQQKPGQPPKLLIKYTSNLESGVPARFSGSGSGTDFILNIHPVEEEDAATYYCQHSRELPWTFGGGTKLEIK

SEQ ID NO: 8 - protein, 447 aa, Artificial Sequence
  Description of Artificial Sequence Synthetic polypeptide
QVQLVQSGAEVKKPGASVKVSCKGSGYTFTDYGMHWVRQAPGQGLEWMGVISTYNGYTNYNQKFKGRVTMTVDKSTSTAYMELRSLRSDDTAVYYCARAYYGNLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSAYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - protein, 218 aa, Artificial Sequence
  Description of Artificial Sequence Synthetic polypeptide
DIVLTQSPASLAVSLGQRATISCRASKSVSTSSYSYMHWYQQKPGQPPKLLIKYASNLESGVPARFSGSGSGTDFSLNIHPMEEDDTAMYFCQHSRELPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 129 aa, Macaca fascicularis
MARGSLRRLLRLLVLGLWLALLRSVAGEQAPGTAPCSHGSSWSADLDKCMDCASCRARPHSDFCLGCSAAPPAPFRLLWPILGGALSLTFVLGLLSGFLVWRRCRRREKFTTPIEETGGEGCPAVALIQ

SEQ ID NO: 11 - protein, 121 aa, Artificial Sequence
  Description of Artificial Sequence Synthetic polypeptide
QVQLVQSGAEVKKPGASVKVSCKGSGYTFTDYGMHWVRQAPGQGLEWMGVISTYNGYTNYNQKFKGRVTMTVDKSTSTAYMELRSLRSDDTAVYYCARAYYGNLYYAMDYWGQGTLVTVSS

SEQ ID NO: 12 - protein, 121 aa, Artificial Sequence
  Description of Artificial Sequence Synthetic polypeptide
QVQLVQSGAEVKKPGASVKVSCKGSGYTFTDYGMHWVRQAPGQGLEWIGVISTYNGYTNYNQKFKGRATMTVDKSTSTAYMELRSLRSDDTAVYYCARAYYGNLYYAMDYWGQGTLVTVSS

SEQ ID NO: 13 - protein, 111 aa, Artificial Sequence
  Description of Artificial Sequence Synthetic polypeptide
DIVLTQSPASLAVSLGQRATISCRASKSVSTSSYSYMHWYQQKPGQPPKLLIKYASNLESGVPARFSGSGSGTDFSLNIHPMEEDDTAMYFCQHSRELPFTFGGGTKLEIK

SEQ ID NO: 14 - protein, 111 aa, Artificial Sequence
  Description of Artificial Sequence Synthetic polypeptide
DIVLTQSPASLAVSLGQRATISCRASKSVSTSSYSYMHWYQQKPGQPPKLLIKYASNLESGVPARFSGSGSGTDFILNIHPMEEDDTAMYFCQHSRELPFTFGGGTKLEIK

SEQ ID NO: 15 - protein, 111 aa, Artificial Sequence
  Description of Artificial Sequence Synthetic polypeptide
DIVLTQSPASLAVSLGQRATISCRASKSVSTSSYSYMHWYQQKPGQPPKLLIKYASNLESGVPARFSGSGSGTDFILNIHPMEEDDTATYYCQHSRELPFTFGGGTKLEIK

SEQ ID NO: 16 - protein, 450 aa, Artificial Sequence
  Description of Artificial Sequence Synthetic polypeptide
QVQLVQSGAEVKKPGASVKVSCKGSGYTFTDYGMHWVRQAPGQGLEWMGVISTYNGYTNYNQKFKGRVTMTVDKSTSTAYMELRSLRSDDTAVYYCARAYYGNLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - DNA, 363 bp, Artificial Sequence
  Description of Artificial Sequence Synthetic polynucleotide
caggtccagctgcagcagtctgggcctgaggtggtgaggcctggggtctcagtgaagatttcctgcaagggttccggctacacattcactgattatggtatgcactgggtgaagcagagtcatgcaaagagtctagagtggattggagttattagtacttacaatggttatacaaactacaaccagaagtttaagggcaaggccacaatgactgtagacaaatcctccagcacagcctatatggaacttgccagattgacatctgaggattctgccatctattactgtgcaagagcctactatggtaacctttactatgctatggactactggggtcaaggaacctcagtcaccgtctcctca

SEQ ID NO: 18 - DNA, 363 bp, Artificial Sequence
  Description of Artificial Sequence Synthetic polynucleotide
caggtccagctgcagcagtctgggcctgaggtggtgaggcctggggtctcagtgaagatttcctgcaagggttccggctacacattcactgattatggtatacactgggtgaagcagagtcatgcaaagagtctagagtggattggagttattagtacttacaatggttatacaaactacaaccagaagtttaagggcaaggccacaatgactgtagacaaatcctccagcacagcctatatggaacttgccagattgacatctgaggattctgccatctattactgtgcaagagcctactatggtaacctttactatgctatggactactggggtcaaggaacctcagtcaccgtctcctca

SEQ ID NO: 19 - DNA, 368 bp, Artificial Sequence
  Description of Artificial Sequence Synthetic polynucleotide
caggtttctctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagaaaccaggttttcctcaagatcaccagtgtggacactgcagatactgccacatactactgtgctcgaaggggacccgattactacggctactatcctatggactactggggtcaaggaacctcagtcaccgtctcctc

SEQ ID NO: 20 - DNA, 333 bp, Artificial Sequence
  Description of Artificial Sequence Synthetic polynucleotide
gacattgtgctgacacagtctcctgcttccttagctgtatctctggggcagagggccaccatctcatgcagggccagcaaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcatcctcaacatccatccagtggaggaggaggatgctgcaacctattactgtcagcacagtagggagcttccattcacgttcggctcggggacaaagttggaaataaaa

SEQ ID NO: 21 - DNA, 333 bp, Artificial Sequence
  Description of Artificial Sequence Synthetic polynucleotide
gacattgtgctgacacagtctcctgcttccttagctgtatctctggggcagagggccaccatctcatgcagggccaacaaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcatcctcaacatccatccagtggaggaggaggatgctgcaacctattactgtcagcacagtagggagcttccattcacgttcggctcggggacaaagttggaaataaaa

SEQ ID NO: 22 - DNA, 333 bp, Artificial Sequence
  Description of Artificial Sequence Synthetic polynucleotide
gacattgtgctgacacagtctcctgcttccttagctgtatctctggggcagagggccaccatctcatgcagggccagcaaaagtgtcagtacatctagctatagttatatgcactggtaccaacagaaaccaggacagccacccaaactcctcatcaagtatacatccaacctagaatctggggtccctgccaggttcagtggcagtgggtctgggacagacttcatcctcaacatccatccagtggaggaggaggatgctgcaacctattactgtcagcacagtagggagcttccgtggacgttcggtggaggcaccaagctggaaatcaaa

SEQ ID NO: 23 - DNA, 363 bp, Artificial Sequence
  Description of Artificial Sequence Synthetic polynucleotide
caggtccagctggtgcagtctggggctgaggtgaagaagcctggggcctcagtgaaggtttcctgcaagggttccggctacacattcactgattatggcatgcactgggtgcggcaggcccctggacaagggctagagtggatgggagttattagtacttacaatggttatacaaactacaaccagaagtttaagggcagagtcacaatgactgtagacaaatccacgagcacagcctatatggaacttcggagcttgagatctgacgatacggccgtgtattactgtgcaagagcctactatggcaacctttactatgctatggactactggggtcaaggaaccctggtcaccgtctcctca

SEQ ID NO: 24 - DNA, 363 bp, Artificial Sequence
  Description of Artificial Sequence Synthetic polynucleotide
caggtccagctggtgcagtctggggctgaggtgaagaagcctggggcctcagtgaaggtttcctgcaagggttccggctacacattcactgattatggcatgcactgggtgcggcaggcccctggacaagggctcgagtggatcggagttattagtacttacaatggttatacaaactacaaccagaagtttaagggaagagccacaatgactgtagacaaatccacgagcacagcctatatggaacttcggagcttgagatctgacgatacggccgtgtattactgtgcaagagcctactatggcaacctttactatgctatggactactggggtcaaggaaccctggtcaccgtctcctca

SEQ ID NO: 25 - DNA, 333 bp, Artificial Sequence
  Description of Artificial Sequence Synthetic polynucleotide
gacattgtgctgacacagtctcctgcttccctggctgtatctctggggcagagggccaccatctcatgcagggccagcaaaagtgtcagtacatctagctatagttatatgcactggtaccaacagaaaccaggacagccacccaaactcctcatcaaatatgcatccaacctagaatctggggtccctgccaggttcagtggcagtgggtctgggacagacttctccctcaacatccatcccatggaggaggacgataccgcaatgtatttctgtcagcacagtagggagcttccattcacgttcggcggagggacaaagttggaaataaaa

SEQ ID NO: 26 - DNA, 333 bp, Artificial Sequence
  Description of Artificial Sequence Synthetic polynucleotide
gacattgtgctgacacagtctcctgcttccctggctgtatctctggggcagagggccaccatctcatgcagggccagcaaaagtgtcagtacatctagctatagttatatgcactggtaccaacagaaaccaggacagccacccaaactcctcatcaaatatgcatccaacctagaatctggggtccctgccaggttcagtggcagtgggtctgggacagacttcatcctcaacatccatccaatggaggaggacgataccgcaatgtatttctgtcagcacagtagggagcttccattcacgttcggcggagggacaaagttggaaataaaa

SEQ ID NO: 27 - DNA, 333 bp, Artificial Sequence
  Description of Artificial Sequence Synthetic polynucleotide
gacattgtgctgacacagtctcctgcttccctggctgtatctctggggcagagggccaccatctcatgcagggccagcaaaagtgtcagtacatctagctatagttatatgcactggtaccaacagaaaccaggacagccacccaaactcctcatcaaatatgcatccaacctagaatctggggtccctgccaggttcagtggcagtgggtctgggacagacttcatcctcaacatccatccaatggaggaggacgataccgcaacctattactgtcaacacagtagggagcttccattcacgttcggcggagggacaaagttggaaataaaa

SEQ ID NO: 28 - protein, 129 aa, Mus musculus
MASAWPRSLPQILVLGFGLVLMRAAAGEQAPGTSPCSSGSSWSADLDKCMDCASCPARPHSDFCLGCAAAPPAHFRLLWPILGGALSLVLVLALVSSFLVWRRCRRREKFTTPIEETGGEGCPGVALIQ

SEQ ID NO: 29 - protein, 129 aa, Rattus norvegicus
MAPGWPRPLPQLLVLGFGLVLIRATAGEQAPGNAPCSSGSSWSADLDKCMDCASCPARPHSDFCLGCAAAPPAHFRMLWPILGGALSLALVLALVSGFLVWRRCRRREKFTTPIEETGGEGCPGVALIQ

SEQ ID NO: 30 - protein, 129 aa, Artificial Sequence
  Description of Artificial Sequence Synthetic consensuspolypeptide
MARGXXRRLXXLLVLGXXLXLLRXVAGEQAPGTAPCSSGSSWSADLDKCMDCASCXARPHSDFCLGCAAAPPAXFRLLWPILGGALSLTXVLXLXSGFLVWRRCRRREKFTTPIEETGGEGCPXVALIQ

SEQ ID NO: 31 - protein, 120 aa, Xenopus laevis
MTPRNLLRTFVPLLLLVLSSAASQGECPEGRAYSQDLGKCMECSVCKNSEKSDFCQNCPSKTEQPDFPWIWVIGFSAGGVFLIIVILSLTVYLTHCRRKSKFTTPIEETGSHSAEALLIH

SEQ ID NO: 32 - DNA, 1410 bp, Artificial Sequence
  Description of Artificial Sequence Synthetic polynucleotide
atgggatgcagctgggtcatgctctttctggtagcaacagctacaggtgtgcactcccaggtccagctgcagcagtctgggcctgaggtggtgaggcctggggtctcagtgaagatttcctgcaagggttccggctacacattcactgattatggtatgcactgggtgaagcagagtcatgcaaagagtctagagtggattggagttattagtacttacaatggttatacaaactacaaccagaagtttaagggcaaggccacaatgactgtagacaaatcctccagcacagcctatatggaacttgccagattgacatctgaggattctgccatctattactgtgcaagagcctactatggtaacctttactatgctatggactactggggtcaaggaacctcagtcaccgtctcctcagcctcaacgaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 33 - protein, 450 aa, Artificial Sequence
  Description of Artificial Sequence Synthetic polypeptide
QVQLQQSGPEVVRPGVSVKISCKGSGYTFTDYGMHWVKQSHAKSLEWIGVISTYNGYTNYNQKFKGKATMTVDKSSSTAYMELARLTSEDSAIYYCARAYYGNLYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - DNA, 717 bp, Artificial Sequence
  Description of Artificial Sequence Synthetic polynucleotide
atggagacagacacactcctgctatgggtactgctgctctgggttccaggttccactggtgacattgtgctgacacagtctcctgcttccttagctgtatctctggggcagagggccaccatctcatgcagggccagcaaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcatcctcaacatccatccagtggaggaggaggatgctgcaacctattactgtcagcacagtagggagcttccattcacgttcggctcggggacaaagttggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 35 - protein, 218 aa, Artificial Sequence
  Description of Artificial Sequence Synthetic polypeptide
DIVLTQSPASLAVSLGQRATISCRASKSVSTSSYSYMHWYQQKPGQPPKLLIKYASNLESGVPARFSGSGSGTDFILNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 1410 bp, Artificial Sequence
  Description of Artificial Sequence Synthetic polynucleotide
atgggatgcagctgggtcatgctctttctggtagcaacagctacaggcgtgcactcccaggtccagctggtgcagtctggggctgaggtgaagaagcctggggcctcagtgaaggtttcctgcaagggttccggctacacattcactgattatggcatgcactgggtgcggcaggcccctggacaagggctagagtggatgggagttattagtacttacaatggttatacaaactacaaccagaagtttaagggcagagtcacaatgactgtagacaaatccacgagcacagcctatatggaacttcggagcttgagatctgacgatacggccgtgtattactgtgcaagagcctactatggcaacctttactatgctatggactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 37 - protein, 450 aa, Artificial Sequence
  Description of Artificial Sequence Synthetic polypeptide
QVQLVQSGAEVKKPGASVKVSCKGSGYTFTDYGMHWVRQAPGQGLEWMGVISTYNGYTNYNQKFKGRVTMTVDKSTSTAYMELRSLRSDDTAVYYCARAYYGNLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - DNA, 1410 bp, Artificial Sequence
  Description of Artificial Sequence Synthetic polynucleotide
atgggatgcagctgggtcatgctctttctggtagcaacagctacaggcgtgcactcccaggtccagctggtgcagtctggggctgaggtgaagaagcctggggcctcagtgaaggtttcctgcaagggttccggctacacattcactgattatggcatgcactgggtgcggcaggcccctggacaagggctcgagtggatcggagttattagtacttacaatggttatacaaactacaaccagaagtttaagggaagagccacaatgactgtagacaaatccacgagcacagcctatatggaacttcggagcttgagatctgacgatacggccgtgtattactgtgcaagagcctactatggcaacctttactatgctatggactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 39 - protein, 450 aa, Artificial Sequence
  Description of Artificial Sequence Synthetic polypeptide
QVQLVQSGAEVKKPGASVKVSCKGSGYTFTDYGMHWVRQAPGQGLEWIGVISTYNGYTNYNQKFKGRATMTVDKSTSTAYMELRSLRSDDTAVYYCARAYYGNLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - DNA, 717 bp, Artificial Sequence
  Description of Artificial Sequence Synthetic polynucleotide
atggagacagacacactcctgctatgggtactgctgctctgggttcctggttccactggtgacattgtgctgacacagtctcctgcttccctggctgtatctctggggcagagggccaccatctcatgcagggccagcaaaagtgtcagtacatctagctatagttatatgcactggtaccaacagaaaccaggacagccacccaaactcctcatcaaatatgcatccaacctagaatctggggtccctgccaggttcagtggcagtgggtctgggacagacttcatcctcaacatccatccaatggaggaggacgataccgcaatgtatttctgtcagcacagtagggagcttccattcacgttcggcggagggacaaagttggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 41 - protein, 218 aa, Artificial Sequence
  Description of Artificial Sequence Synthetic polypeptide
DIVLTQSPASLAVSLGQRATISCRASKSVSTSSYSYMHWYQQKPGQPPKLLIKYASNLESGVPARFSGSGSGTDFILNIHPMEEDDTAMYFCQHSRELPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - DNA, 717 bp, Artificial Sequence
  Description of Artificial Sequence Synthetic polynucleotide
atggagacagacacactcctgctatgggtactgctgctctgggttcctggttccactggtgacattgtgctgacacagtctcctgcttccctggctgtatctctggggcagagggccaccatctcatgcagggccagcaaaagtgtcagtacatctagctatagttatatgcactggtaccaacagaaaccaggacagccacccaaactcctcatcaaatatgcatccaacctagaatctggggtccctgccaggttcagtggcagtgggtctgggacagacttctccctcaacatccatcccatggaggaggacgataccgcaatgtatttctgtcagcacagtagggagcttccattcacgttcggcggagggacaaagttggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 43 - protein, 218 aa, Artificial Sequence; identical to SEQ ID NO: 9
  Description of Artificial Sequence Synthetic polypeptide

SEQ ID NO: 44 - DNA, 717 bp, Artificial Sequence
  Description of Artificial Sequence Synthetic polynucleotide
atggagacagacacactcctgctatgggtactgctgctctgggttcctggttccactggtgacattgtgctgacacagtctcctgcttccctggctgtatctctggggcagagggccaccatctcatgcagggccagcaaaagtgtcagtacatctagctatagttatatgcactggtaccaacagaaaccaggacagccacccaaactcctcatcaaatatgcatccaacctagaatctggggtccctgccaggttcagtggcagtgggtctgggacagacttcatcctcaacatccatccaatggaggaggacgataccgcaacctattactgtcaacacagtagggagcttccattcacgttcggcggagggacaaagttggaaataaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 45 - protein, 218 aa, Artificial Sequence
  Description of Artificial Sequence Synthetic polypeptide
DIVLTQSPASLAVSLGQRATISCRASKSVSTSSYSYMHWYQQKPGQPPKLLIKYASNLESGVPARFSGSGSGTDFILNIHPMEEDDTATYYCQHSRELPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 1401 bp, Artificial Sequence
  Description of Artificial Sequence Synthetic polynucleotide
atgggatgcagctgggtcatgctctttctggtagcaacagctacaggcgtgcactcccaggtccagctggtgcagtctggggctgaggtgaagaagcctggggcctcagtgaaggtttcctgcaagggttccggctacacattcactgattatggcatgcactgggtgcggcaggcccctggacaagggctagagtggatgggagttattagtacttacaatggttatacaaactacaaccagaagtttaagggcagagtcacaatgactgtagacaaatccacgagcacagcctatatggaacttcggagcttgagatctgacgatacggccgtgtattactgtgcaagagcctactatggcaacctttactatgctatggactactggggtcaaggaaccctggtcaccgtctcctcagcctccaccaagggcccatccgtcttccccctggcgccctgctccagatctacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcgcgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaagtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtcctcgattccgacggctccttcttcctctacagcaggctaaccgtggacaagagcaggtggcaggaggggaatgtcttctcatgctccgtgatgcatgaggctctgcacaaccactacacacagaagagcctctccctgtctctgggttga

SEQ ID NO: 47 - DNA, 1410 bp, Artificial Sequence
  Description of Artificial Sequence Synthetic polynucleotide
atgggatgcagctgggtcatgctctttctggtagcaacagctacaggcgtgcactcccaggtccagctggtgcagtctggggctgaggtgaagaagcctggggcctcagtgaaggtttcctgcaagggttccggctacacattcactgattatggcatgcactgggtgcggcaggcccctggacaagggctagagtggatgggagttattagtacttacaatggttatacaaactacaaccagaagtttaagggcagagtcacaatgactgtagacaaatccacgagcacagcctatatggaacttcggagcttgagatctgacgatacggccgtgtattactgtgcaagagcctactatggcaacctttactatgctatggactactggggtcaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcgcgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctcccggttga

SEQ ID NO: 48 - protein, 450 aa, Artificial Sequence; identical to SEQ ID NO: 16
  Description of Artificial Sequence Synthetic polypeptide

SEQ ID NO: 49 - protein, 69 aa, Artificial Sequence
  Description of Artificial Sequence Synthetic polypeptide
EQAPGTAPCSRGSSWSADLDKCMDCASCRARPHSDFCLGCAAAPPAPFRLLWPEQKLISEEDLHHHHHH

SEQ ID NO: 50 - protein, 15 aa, Artificial Sequence
  Description of Artificial Sequence Synthetic peptide
RASKSVSTSSYSYMH

SEQ ID NO: 51 - protein, 7 aa, Artificial Sequence
  Description of Artificial Sequence Synthetic peptide
YASNLES

SEQ ID NO: 52 - protein, 7 aa, Artificial Sequence
  Description of Artificial Sequence Synthetic peptide
SRELPFT

SEQ ID NO: 53 - protein, 10 aa, Artificial Sequence
  Description of Artificial Sequence Synthetic peptide
GYTFTDYGMH

SEQ ID NO: 54 - protein, 17 aa, Artificial Sequence
  Description of Artificial Sequence Synthetic peptide
VISTYNGYTNYNQKFKG

SEQ ID NO: 55 - protein, 12 aa, Artificial Sequence
  Description of Artificial Sequence Synthetic peptide
AYYGNLYYAMDY

SEQ ID NO: 56 - protein, 53 aa, Homo sapiens
EQAPGTAPCSRGSSWSADLDKCMDCASCRARPHSDFCLGCAAAPPAPFRLLWP

SEQ ID NO: 57 - protein, 53 aa, Macaca fascicularis
EQAPGTAPCSHGSSWSADLDKCMDCASCRARPHSDFCLGCSAAPPAPFRLLWP

SEQ ID NO: 58 - protein, 53 aa, Mus musculus
EQAPGTSPCSSGSSWSADLDKCMDCASCPARPHSDFCLGCAAAPPAHFRLLWP

SEQ ID NO: 59 - protein, 49 aa, Xenopus laevis
QGECPEGRAYSQDLGKCMECSVCKNSEKSDFCQNCPSKTEQPDFPWIWV